(19) United States
(12) Patent Application Publication
(10) Pub. No.: US 2011/0183848 A1
(43) Pub. Date: Jul. 28, 2011

(54) Piperazine Compounds With Herbicidal Effect

(75) Inventors: Liliana Parra Rapado, Offenburg (DE); Frank Stelzer, Mannheim (DE); Matthias Witschel, Bad Duerheim (DE); Thomas Seitz, Viernheim (DE); Anja Simon, Weinheim (DE); Robert Reinhard, Limburgerhof (DE); Bernd Sievernich, Hassloch (DE); Klaus Grossmann, Neuhofen (DE); Thomas Ehrhardt, Speyer (DE); Trevor Wlliam Newton, Neustadt (DE); Julia Major, Mannheim (DE); Tao Qu, Ludwigshafen (DE); William Karl Moberg, Hassloch (DE); Dschun Song, Mannheim (DE); Michael Rack, Eppelheim (DE); Timo Frassetto, Mannheim (DE); Klaus Kreuz, Denzlingen (DE)

(73) Assignee: BASF SE, Ludwigshafen (DE)

(21) Appl. No.: 13/121,976

(22) PCT Filed: Sep. 29, 2009
(86) PCT No.: PCT/EP2009/062573

§ 371 Date: Mar. 31, 2011

(30) Foreign Application Priority Data
Oct. 2, 2008 (EP) 08165693.6

(51) Int. Cl. A01N 43/70; C07D 241/08; A01N 43/60; A01P 13/00; C07D 401/06; A01N 55/10; C07F 7/08

(52) U.S. Cl. 504/134; 544/383; 504/235; 504/136; 544/360; 504/193; 544/229

(57) ABSTRACT

Piperazine compounds of the formula I

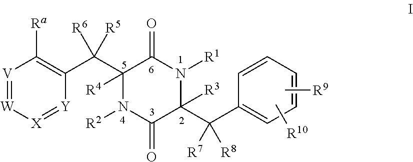

in which the variables are defined according to the description, their agriculturally suitable salts, processes and intermediates for preparing the piperazine compounds of the formula I, compositions comprising them and their use as herbicides, i.e. for controlling harmful plants, and also a method for controlling unwanted vegetation which comprises allowing a herbicidally effective amount of at least one piperazine compound of the formula I to act on plants, their seed and/or their habitat.

The present invention relates to piperazine compounds of the formula I

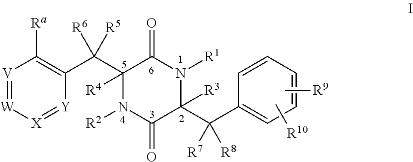

in which the variables are as defined below:
R^{a} is halogen, CN, NO₂, C₁-C₄-alkyl, C₃-C₆-cycloalkyl, C₁-C₄-haloalkyl, C₁-C₄-alkoxy, C₁-C₄-thioalkyl, C₁-C₄-haloalkoxy, C₁-C₄-halothioalkyl, O—Z—C₃-C₆-cycloalkyl, S(O)ₙR^{y}, C₂-C₆-alkenyl, C₃-C₆-cycloalkenyl, C₃-C₆-alkenyloxy, C₃-C₆-thioalkenyl, C₂-C₆-alkynyl, C₃-C₆-alkynyloxy, C₃-C₆-thioalkynyl, NR^{A}R^{B}, tri-C₁-C₄-alkylsilyl, Z—C(═O)—R^{a1}, Z—C(═S)—R^{a1}, Z—C(═N—OR^{A})—R^{a1}, Z—C[═N(O)—R^{A}]—R^{a1}, Z—P(═O)(R^{a1})₂, a 3- to 7-membered monocyclic or 9- or 10-membered bicyclic saturated, unsaturated or aromatic heterocycle which is attached via carbon or nitrogen, which contains 1, 2, 3 or 4 heteroatoms selected from the group consisting of O, N and S and which may be partially or fully substituted by groups R^{aa} and/or R^{a1} and/or may be fused to a further saturated, unsaturated or aromatic carbocyclic or heterocyclic ring,
R^{y} is C₁-C₆-alkyl, C₃-C₄-alkenyl, C₃-C₄-alkynyl, NR^{A}R^{B} or C₁-C₄-haloalkyl and n is 0, 1 or 2;
R^{A}, R^{B} independently of one another are hydrogen, C₁-C₆-alkyl, C₃-C₆-alkenyl or C₃-C₆-alkynyl, C₃-C₆-cycloalkyl, C₁-C₆-alkylcarbonyl, C₃-C₆-cycloalkylcarbonyl, C₃-C₆-alkenylcarbonyl, C₃-C₆-cycloalkenylcarbonyl and C₃-C₆-alkynylcarbonyl
R^{A}, R^{B} together with the nitrogen atom to which they are attached may also form a five- or six-membered saturated or partially or fully unsaturated ring which, in addition to carbon atoms, may contain 1, 2 or 3 heteroatoms selected from the group consisting of O, N and S, which ring may be substituted by 1 to 3 groups R^{aa};
Z is a covalent bond, C₁-C₄-alkylene, C₂-C₆-alkenyl or C₂-C₆-alkynyl;
R^{a1} is hydrogen, OH, C₁-C₈-alkyl, C₁-C₄-haloalkyl, C₃-C₆-cycloalkyl, C₂-C₈-alkenyl, C₅-C₆-cycloalkenyl, C₂-C₈-alkynyl, C₁-C₆-alkoxy, C₁-C₄-haloalkoxy, C₃-C₈-alkenyloxy, C₃-C₈-alkynyloxy, NH₂, C₁-C₆-alkylamino, [di-(C₁-C₆)-alkyl]amino, C₁-C₆-alkoxyamino, C₁-C₆-alkylsulfonylamino, C₁-C₆-alkyl-aminosulfonylamino, [di-(C₁-C₆)-alkylamino]sulfonylamino, C₃-C₆-alkenyl-amino, C₃-C₆-alkynylamino, N—(C₂-C₆-alkenyl)-N—(C₁-C₆-alkyl)amino, N—(C₂-C₆-alkynyl)-N—(C₁-C₆-alkyl)amino, N—(C₁-C₆-alkoxy)-N—(C₁-C₆-alkyl)amino, N—(C₂-C₆-alkenyl)-N—(C₁-C₆-alkoxy)amino, N—(C₂-C₆-alkynyl)-N—(C₁-C₆-alkoxy)amino, C₁-C₆-alkylsulfonyl, tri-C₁-C₄-alkylsilyl, phenyl, phenoxy, phenylamino or a 5- or 6-membered monocyclic or 9- or 10-membered bicyclic heterocycle which contains 1, 2, 3 or 4 heteroatoms selected from the group consisting of O, N and S, where the cyclic groups are unsubstituted or substituted by 1, 2, 3 or 4 groups R^{aa};
R^{aa} is halogen, OH, CN, NO₂, C₁-C₄-alkyl, C₁-C₄-haloalkyl, C₁-C₄-alkoxy, C₁-C₄-haloalkoxy, S(O)ₙR^{y}, Z—C(═O)—R^{a1}, Z—C(═S)—R^{a1}, Z—C(═N—OR^{A})—R^{a1}, Z—C(═N(O)—R^{A})—R^{a1}, oxo (═O) and tri-C₁-C₄-alkylsilyl;
R¹ is OH, CN, C₅-C₁₂-alkyl, C₅-C₁₂-alkenyl, C₅-C₁₂-alkynyl, C₁-C₄-alkoxy, C₃-C₆-cycloalkyl, C₅-C₆-cycloalkenyl, CH₂CH═CHCH₃, CH₂CH₂CH═CH₂, CH₂C(CH₃)═CH₂, CH₂CH═C(CH₃)₂, CH₂C≡CCH₃, CH₂CH₂C≡CH, NR^{A}R^{B}, S(O)ₙR^{y}, S(O)ₙ, NR^{A}R^{B}, CO—C₁-C₂-chloroalkyl, CONR^{A}R^{B}, phenyl or a 5- or 6-membered monocyclic or 9- or 10-membered bicyclic saturated, partially unsaturated or aromatic heterocycle, which contains 1, 2, 3 or 4 heteroatoms selected from the group consisting of O, N and S, where the cyclic groups are attached via Z¹ and are unsubstituted or substituted by 1, 2, 3 or 4 groups R^{aa}, and also the following partially or fully R^{aa}-substituted groups: C₁-C₄-alkyl, C₃-C₄-alkenyl and C₃-C₄-alkynyl;
Z¹ is carbonyl or a group Z;

where in groups R¹, R^{a} and their sub-substituents the carbon chains and/or the cyclic groups may carry 1, 2, 3 or 4 substituents R^{aa} and/or R^{a1},
R² is C₁-C₄-alkyl, C₃-C₄-alkenyl or C₃-C₄-alkynyl;
R³ is OH, NH₂, C₁-C₄-alkyl, C₃-C₆-cycloalkyl, C₃-C₆-alkenyl, C₃-C₆-alkynyl, C₁-C₄-hydroxyalkyl, C₁-C₄-cyanoalkyl, C₁-C₄-haloalkyl, C₁-C₄-alkoxy-C₁-C₄-alkyl or C(═O)R¹¹;

R¹¹ is hydrogen, C₁-C₄-alkyl, C₁-C₄-haloalkyl, C₁-C₄-alkoxy or C₁-C₄-haloalkoxy;
R⁴ is hydrogen, halogen, C₁-C₄-alkyl or C₁-C₄-haloalkyl, or R⁴ and R⁵ together are a covalent bond;
R⁵, R⁶, R⁷, R⁵ independently of one another are hydrogen, halogen, OH, CN, NO₂, C₁-C₄-alkyl, C₁-C₄-haloalkyl, C₂-C₆-alkenyl, C₂-C₆-alkynyl, C₁-C₄-alkoxy, C₁-C₄-haloalkoxy, C₃-C₆-cycloalkyl, C₃-C₆-cycloalkenyl or C₃-C₆-cycloalkynyl;
R⁹, R¹⁹ independently of one another are hydrogen, halogen, OH, haloalkyl, NR^{A}R^{B}, NRAC(O)R⁹¹, CN, NO₂, C₁-C₄-alkyl, C₁-C₄-haloalkyl, C₂-C₄-alkenyl, C₃-C₆-alkynyl, C₁-C₄-alkoxy, C₁-C₄-haloalkoxy, O—C(O)R⁹¹, phenoxy or benzyloxy,

where in groups R⁹ and R¹⁹ the carbon chains and/or the cyclic groups may carry 1, 2, 3 or 4 substituents R^{aa};

R⁹¹ is C₁-C₄-alkyl or NR^{A}R^{B};
V, W, X, Y are independently of one another N or C—R^{b},
R^{b} independently of one another are hydrogen, CN, NO₂, halogen, C₁-C₄-alkyl, C₁-C₄-haloalkyl, C₂-C₄-alkenyl, C₃-C₆-alkynyl, C₁-C₄-alkoxy, C₁-C₄-haloalkoxy, benzyl or S(O)ₙR^{y};

R^{a} and/or R^{b} together with the group R^{a} or R^{b} attached to the adjacent ring carbon atom or with the adjacent ring nitrogen atom itself may also form a five- or six-membered saturated or partially or fully unsaturated ring which, in addition to carbon atoms, may contain 1, 2 or 3 heteroatoms selected from the group consisting of O, N and S, which ring may be substituted by 1 to 3 groups R^{aa} and/or may be fused to a further saturated, unsaturated or aromatic carbocyclic or heterocyclic ring;
is a double bond, or, if R^{a} forms a cycle with a nitrogen atom in position V, is a single bond;
   with the proviso that, if from one to three groups of V, W, X, Y are N, R¹ is not
C₅-C₆-alkyl, C₁-C₄-haloalkyl, CH₂CH═CHCH₃, CH₂CH₂CH═CH₂, CH₂C(CH₃)═CH₂, CH₂C≡CCH₃ or CH₂CH₂C≡CH;
   and the agriculturally suitable salts thereof.

Moreover, the invention relates to processes and intermediates for preparing the piperazine compounds of the formula I and the agriculturally usable salts thereof, to compositions comprising them and to their use as herbicides, i.e. for controlling harmful plants, and also to a method for controlling unwanted vegetation which comprises allowing a herbicidally effective amount of at least one piperazine compound of the formula I or of an agriculturally suitable salt of I to act on plants, their seed and/or their habitat.

Further embodiments of the present invention can be found in the claims, the description and the examples. It is to be understood that the features mentioned above and those still to be illustrated below of the subject matter of the invention can be applied not only in the respective given combination but also in other combinations without leaving the scope of the invention.

The thaxtomins A and B (King R. R. et al., J. Agric. Food Chem. (1992) 40, 834-837) produced by the plant pathogen S. scabies are natural products having a central piperazine-2,5-dione ring which carries a 4-nitroindol-3-ylmethyl radical in the 3-position and an optionally OH-substituted benzyl radical in the 2-position. Owing to their plant-damaging action, this compound class has also been examined for suitability for use as herbicides (King R. R. et al., J. Agric. Food Chem. (2001) 49, 2298-2301).

WO 2007/077201 and WO 2007/077247 describe herbicidal 2,5-diketopiperazines which, in the 3- and 6-positions, have phenyl or hetaryl groups attached via methylene or methine groups.

It is an object of the present invention to provide compounds having herbicidal action. To be provided are in particular compounds having strong herbicidal action, in particular even at low application rates, whose compatibility with crop plants is sufficient for commercial application.

These and further objects are achieved by the compounds of the formula I defined at the outset and by their agriculturally suitable salts.

The compounds according to the invention differ from those known from WO 2007/077201 and WO 2007/077247 essentially by the N-substitution in position 1 and the substituent in position 2 of the piperazine ring.

The compounds according to the invention can be prepared analogously to the synthesis routes described in WO 2007/077201 and WO 2007/077247 according to standard processes of organic chemistry, for example a process (hereinbelow process A) which comprises the steps below:

Process A

Compounds of the formula I where R¹≠hydrogen can preferably be prepared by reacting a piperazine compound of the formula I in which R¹ is hydrogen with an alkylating agent or acylating agent which contains a group R¹ different from hydrogen (process A). Such reactions can be carried out analogously to known processes, for example according to the methods described by I. O. Donkor et al., Bioorg. Med. Chem. Lett. 11 (19) (2001), 2647-2649, B. B. Snider et al., Tetrahedron 57 (16) (2001), 3301-3307, I. Yasuhiro et al., J. Am. Chem. Soc. 124(47) (2002), 14017-14019 or M. Falorni et al., Europ. J. Org. Chem. (8) (2000), 1669-1675.

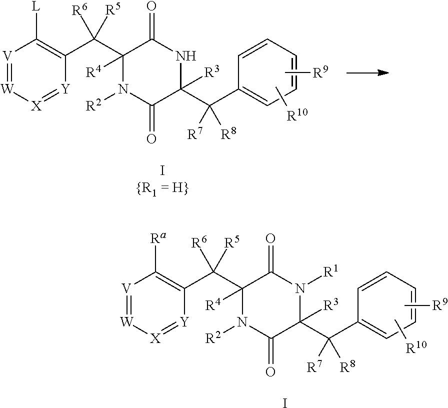

According to process A, a piperazine compound of the formula I where R¹=hydrogen is reacted with a suitable alkylating agent, hereinbelow compound X¹—R¹, or acylating agent, hereinbelow compound X²—R¹, which gives a piperazine compound of the formula I where R¹≠hydrogen.

In the alkylating agents X¹—R¹, X¹ may be halogen or O—SO₂—R^{m}, where R^{m} has the meaning of C₁-C₄-alkyl or aryl which are optionally substituted by halogen, C₁-C₄-alkyl or halo-C₁-C₄-alkyl. In acylating agents X²—R¹, X² may be halogen, in particular Cl. Here, R¹≠hydrogen and has the meaning given above and is in particular C₁-C₆-alkyl, C₃-C₆-cycloalkyl, C₃-C₆-alkenyl, C₃-C₆-cycloalkenyl, C₃-C₆-alkynyl, C₃-C₆-cycloalkynyl, phenyl-C₁-C₆-alkyl, heterocyclyl, heterocyclyl-C₁-C₆-alkyl; phenyl-[C₁-C₆-alkoxycarbonyl]-C₁-C₆-alkyl or phenylheterocyclyl-C₁-C₆-alkyl; or COR¹, or SO₂R²⁵, where the aliphatic, cyclic or aromatic moieties of R¹ mentioned may be partially or fully halogenated and/or may carry one to three of the groups below: cyano, hydroxyl, C₁-C₄-alkyl, C₃-C₆-cycloalkyl, C₁-C₄-alkoxy, C₁-C₄-alkylthio, [di-(C₁-C₄)-alkyl]amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄)-alkyl]aminocarbonyl or C₁-C₄-alkylcarbonyloxy.

The reaction is usually carried out at temperatures in the range of from −78° C. to the boiling point of the reaction mixture, preferably from −50° C. to 65° C., especially preferably from −30° C. to 65° C. The reaction is generally carried out in a solvent, preferably in an inert organic solvent.

Suitable inert organic solvents include aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as dichloromethane, dichloroethane, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol, tert-butyl alcohol, water and also dimethyl sulfoxide, dimethylformamide and dimethylacetamide, and also morpholine and N-methylmorpholine. It is also possible to use mixtures of the solvents mentioned.

In a preferred embodiment of the invention, the reaction is carried out in a tetrahydrofuran/water mixture, for example using a mixing ratio of 1:10 to 10:1 (parts by volume). In another preferred embodiment toluene, dichloromethane, tetrahydrofuran or dimethylformamide or mixtures thereof are considered. In a particularly preferred embodiment of the invention, the reaction is carried out in tetrahydrofuran.

In a preferred embodiment, the compound I where R¹═H is reacted with the alkylating or acylating agent in the presence of a base.

Suitable bases are, in general, inorganic compounds, such as alkali metal and alkaline earth metal hydroxides, such as lithium hydroxide, sodium hydroxide, potassium hydroxide or calcium hydroxide, an aqueous solution of ammonia, alkali metal or alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, for example lithium diisopropylamide, sodium amide and potassium amide, alkali metal and alkaline earth metal carbonates, such as lithium carbonate, potassium carbonate, cesium carbonate and calcium carbonate, and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, for example tertiary amines, such as trimethylamine, triethylamine, diisopropylethylamine, 2-hydroxypyridine and N-methylpiperidine, pyridine, substituted pyridines, such as collidine, lutidine and 4-dimethylaminopyridine, and also bicyclic amines. It is also possible to use a mixture of different bases.

In one embodiment of the process according to the invention, the reaction of II is carried out in the presence of bases, preferably in the presence of the bases potassium tert-butoxide, 2-hydroxypyridine or an aqueous solution of ammonia or a mixture of these bases. Preferably, only one of these bases is used. In a particularly preferred embodiment, the reaction is carried out in the presence of an aqueous solution of ammonia which may, for example, be from 10 to 50% strength (w/v).

The bases are generally employed in equimolar amounts. They can also be employed in excess or even as solvent. In a preferred embodiment of the process according to the invention, the base is added in an equimolar amount or in an essentially equimolar amount. In a further preferred embodiment, the base used is sodium hydride.

The reaction mixtures obtained by one of the processes according to the invention can be worked up, for example, in a customary manner. This can be effected, for example, by mixing with water, separating the phases and, if appropriate, chromatographic purification of the crude products. Some of the intermediate and end products are obtained in the form of viscous oils which, in general, can be freed from volatile constituents or purified under reduced pressure and at moderately elevated temperature. If the intermediates and end products are obtained as solids, the purification can also be carried out by recrystallization or digestion.

Process B

B.1

Analogously to the procedure described above, compounds I in which R² is hydrogen can be reacted with alkylating agents R²—X¹ or acylating agents R²—X², which gives compounds of the formula I where R²≠hydrogen (process B). The reaction conditions correspond to those of process A. Preferred bases are sodium hydride (NaH), lithium diisopropylamide (LDA) and lithium hexamethyldisilazide (LiHMDS)

B.2

Analogously to the procedure described above, compounds I in which R³ is hydrogen can be reacted with alkylating agents R³—X¹ or acylating agents R³—X², which gives compounds of the formula I where R³≠hydrogen. The reaction conditions correspond to those of process A.

If the group R¹ in formula I or II is hydrogen, an alkylation introduces the group R¹. If the group R¹ in formula I or II is a protective group, this is initially removed, giving a compound in which R¹ is hydrogen into which the group R¹ is introduced by alkylation. If R² in formula I or II is hydrogen, the group R² may be introduced by an alkylation or acylation step. If R¹ and R² are identical, the alkylation or acylation steps can be carried out simultaneously or successively in any order. If the groups R¹, R² and R³ are identical, the group R³ can be introduced simultaneously to the introduction of the groups R¹ and/or R² or subsequent thereto.

Alternatively, the groups R¹, R² and/or R³ may also be introduced into other precursors of the compounds I or II. Thus, for example, compounds IV, VI, VIII, IX, XI and XII in which R¹, R² and/or R³ are hydrogen can be subjected to the reactions described above.

Process C

The compounds of the formula I can be prepared according to the process illustrated in the scheme below by converting the substituent R^{a}, for example analogously to J. Tsuji, Top. Organomet. Chem. (14) (2005), 332 pp. or J. Tsuji, Organic Synthesis with Palladium Compounds (1980), 207 pp.

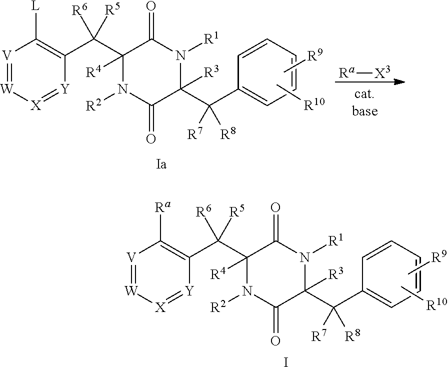

Thus, for example, compounds of the formula I in which R^{a} is CN, optionally substituted phenyl or an optionally substituted heterocyclic group can be prepared from compounds I in which R^{a} is halogen, such as Cl, Br or I, by converting the substituent R^{a}.

To this end, a piperazine compound of the formula Ia which, instead of the substituent R^{a}, has a suitable leaving group L, is converted into another piperazine derivative of the formula I by reaction with a coupling partner which contains a group R^{a} (compound R^{a}—X³).

The reaction is usually carried out in the presence of a catalyst, preferably in the presence of a transition metal catalyst. The reaction is generally carried out in the presence of a base.

Hereinbelow, this reaction sequence is shown using the example of the substituent R^{a}; it is, of course, also possible to utilize this reaction sequence in an analogous manner for converting the substituents R^{b}.

Suitable leaving groups L are, for example, halogen or S(O)ₙR^{k}, where n=0, 1, 2, 3, such as, for example, triflate, where R^{k} has the meaning of C₁-C₆-alkyl, halo-C₁-C₆-alkyl or optionally halogenated or C₁-C₄-alkyl-substituted aryl.

Suitable coupling partners X³—R^{a} are in particular those compounds in which X³, if R^{a} has the meaning of C₁-C₆-alkyl, C₂-C₆-alkenyl, aryl or heteroaryl, is one of the groups below:
Zn—R¹, where IR¹, has the meaning of halogen, C₁-C₆-alkyl, C₂-C₆-alkenyl, aryl or heteroaryl;
B(OR^{m})₂, where R^{m} has the meaning of H or C₁-C₆-alkyl, where two alkyl substituents together may form a C₂-C₄-alkylene chain; or
SnRⁿ₃, where Rⁿ has the meaning of C₁-C₆-alkyl, aryl or alkoxyalkenyl.

If R^{a} is C₂-C₆-alkynyl, X³ may also be hydrogen.

This reaction is usually carried out at temperatures in the range of from −78° C. to the boiling point of the reaction mixture, preferably from −30° C. to 65° C., especially preferably at temperatures of from 30° C. to 65° C. The reaction is generally carried out in an inert organic solvent in the presence of a base.

Suitable solvents are the compounds quoted for process A. In one embodiment of the process according to the invention, tetrahydrofuran and a catalytic amount of water are used; in another embodiment, tetrahydrofuran is employed on its own.

Suitable bases are the compounds quoted for process A. The bases are generally employed in equimolar amounts. They can also be used in excess or even as solvent.

In a preferred embodiment of the process according to the invention, the base is added in an equimolar amount. In a further preferred embodiment, the base used is triethylamine or cesium carbonate, particularly preferably cesium carbonate.

Suitable catalysts for the process according to the invention are, in principle, compounds of the transition metals Ni, Fe, Pd and Cu. It is possible to use organic or inorganic compounds. Transition metal complexes with various ligands are suitable (cf. Accts. Chem. Res. 2008, 41 (11), 1439-1564, special issue; Angew. Chem. Int. Ed. Engl. 2009, 48, 4114-4133). Examples which may be mentioned are: Pd(PPh₃)₂Cl₂, Pd(OAc)₂, PdCl₂ or Na₂PdCl₄. Here, Ph is phenyl.

To prepare the compound I in which R^{a} is CN, the compound Ia in which L is chlorine, bromine or iodine may also be reacted with copper cyanide analogously to known processes (cf. Organikum, 21. edition 2001, Wiley, p. 404; Tetrahedron Lett. 42, 2001, p. 7473; Org. Lett. 5, 2003, 1785).

These reactions are usually carried out at temperatures in the range of from 100° C. to the boiling point of the reaction mixture, preferably from 100° C. to 250° C. The reaction is generally carried out in an inert organic solvent. Suitable solvents are in particular aprotic polar solvents, for example dimethylformamide, N-methylpyrrolidone, N,N′-dimethylimidazolidin-2-one and dimethylacetamide.

Alternatively, the conversion of the group R^{a} can also be carried out in the precursors of the compound I. Thus, for example, compounds II in which R^{a} is a halogen atom, such as Cl, Br or I, can be subjected to the reaction described above.

Process D

According to the synthesis shown below, the compounds of the formula I can be prepared by coupling piperazine compounds of the formula IV with compounds V. The coupling of IV with V can be achieved analogously to known processes, for example according to G. Porzi, et al., Tetrahedron 9 (19), (1998), 3411-3420 or C. I. Harding et al., Tetrahedron 60 (35), (2004), 7679-7692.

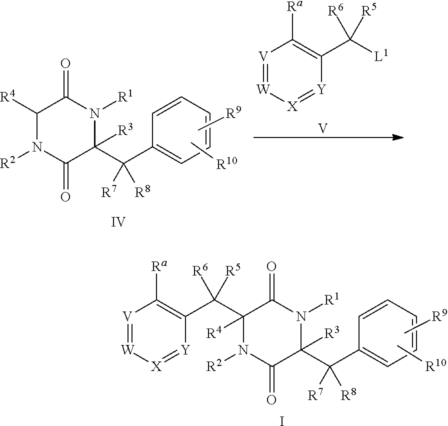

In the scheme, V, W, X, Y and R¹—R¹⁰ have the meanings given above. L¹ is a suitable leaving group, such as halogen or OSO₂R^{m}, where R^{m} has the meaning of C₁-C₄-alkyl, aryl or aryl which is mono- to trisubstituted by C₁-C₄-alkyl.

The reaction is generally carried out at temperatures in the range of from −78° C. to the boiling point of the reaction mixture, preferably in the range of from −78° C. to 40° C., especially preferably in the range of from −78° C. to 30° C.

The reaction is generally carried out in an inert organic solvent in the presence of a base. Suitable solvents are those quoted for process A, preferably tetrahydrofuran.

Suitable bases are the compounds quoted for process A. In a further preferred embodiment, the base used is lithium diisopropylamide, particularly preferably in an essentially equimolar amount, in particular in an equimolar amount.

Some of the compounds of the formula V are commercially available or can be prepared by transformations, described in the literature, of the corresponding commercially available precursors. Work-up can be carried out analogously to process A.

Some of the precursors and intermediates required for preparing the compounds of the formula I are commercially available, known from the literature or can be prepared by processes known from the literature.

The dipeptide compounds of the formula II, for example, can be prepared from N-protected dipeptides of the formula VI analogously to known processes, for example according to Glenn L. Stahl et al., J. Org. Chem. 43(11), (1978), 2285-6 or A. K. Ghosh et al., Org. Lett. 3(4), (2001), 635-638.

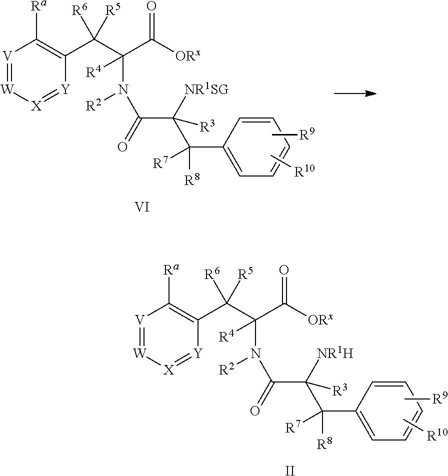

In formulae II and VI, the variables have the meaning given for formula I, SG is a nitrogen protective group, such as Boc (=tert-butoxycarbonyl) and OR^{x} is a leaving group attached via an oxygen atom. The preferred meanings for the compounds of the formula I do, of course, also apply in a corresponding manner to the compounds of the formula II or IV. With respect to the leaving group OR^{x}, what was said above for formula II applies.

Thus, for example, a dipeptide of the formula VI in which SG is Boc and OR^{x} is a suitable leaving group, where R^{x} is, for example, C₁-C₆-alkyl, in particular methyl, ethyl or benzyl, can be converted in the presence of an acid into a compound of the formula II.

The reaction is usually carried out at temperatures in the range of from −30° C. to the boiling point of the reaction mixture, preferably from 0° C. to 50° C., especially preferably from 20° C. to 35° C.

The reaction can be carried out in a solvent, in particular in an inert organic solvent. Suitable solvents are those quoted for the basic cyclization, in particular tetrahydrofuran or dichloromethane or mixtures thereof, preferably dichloromethane.

Suitable acids are, in principle, both Brönstedt and Lewis acids. It is possible to use, in particular, inorganic acids, for example hydrohalic acids, such as hydrofluoric acid, hydrochloric acid, hydrobromic acid, inorganic oxo acids, such as sulfuric acid and perchloric acid, furthermore inorganic Lewis acids, such as boron trifluoride, aluminum trichloride, iron(III) chloride, tin(IV) chloride, titanium(IV) chloride and zinc(II) chloride, and also organic acids, for example carboxylic acids and hydroxycarboxylic acids, such as formic acid, acetic acid, propionic acid, oxalic acid, citric acid and trifluoroacetic acid, and also organic sulfonic acids, such as toluenesulfonic acid, benzenesulfonic acid, camphorsulfonic acid and the like. It is, of course, also possible to use a mixture of different acids.

In one embodiment of the process according to the invention, the reaction is carried out in the presence of organic acids, for example in the presence of strong organic acids, such as formic acid, acetic acid or trifluoroacetic acid or mixtures thereof. In a preferred embodiment, the reaction is carried out in the presence of trifluoroacetic acid. Work-up can be carried out analogously to process A.

The protected dipeptides of the formula VI can be prepared analogously to known processes, for example according to Wilford L. Mendelson et al., Int. J. Peptide & Protein Research 35(3), (1990), 249-57. A typical path is the amidation of a Boc-protected amino acid VIII with an amino acid ester of the formula VII according to the scheme below:

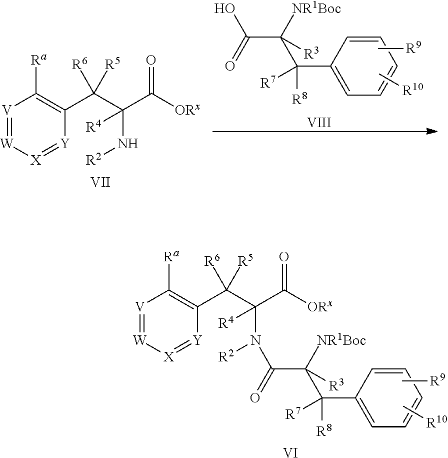

In this scheme, the variables have the meanings mentioned above. Instead of Boc, it is also possible to use other amino protective groups.

The reaction of VII with VIII is generally carried out at temperatures in a range of from −30° C. to the boiling point of the reaction mixture, preferably from 0° C. to 50° C., especially preferably from 20° C. to 35° C. The reaction can be carried out in a solvent, preferably in an inert organic solvent. Suitable are the solvents mentioned for process A in connection with the basic cyclization.

In general, the reaction requires the presence of an activating agent. Suitable activating agents are condensing agents, such as, for example, polystyrene-supported or non-polystyrene-supported dicyclohexylcarbodiimide (DCC), diisopropylcarbodiimide, 1-ethyl-3-(dimethylaminopropyl)carbodiimide (EDAC), carbonyldiimidazole, chloroformic esters, such as methyl chloroformate, ethyl chloroformate, isopropyl chloroformate, isobutyl chloroformate, sec-butyl chloroformate or allyl chloroformate, pivaloyl chloride, polyphosphoric acid, propanephosphonic anhydride, bis(2-oxo-3-oxazolidinyl)phosphoryl chloride (BOPCI) or sulfonyl chlorides, such as methanesulfonyl chloride, toluenesulfonyl chloride or benzenesulfonyl chloride. According to one embodiment, the activating agent used is EDAC or DCC.

The reaction of VII with VIII is preferably carried out in the presence of a base. Suitable bases are the compounds quoted for process A. In one embodiment, the base used is triethylamine or N-ethyldiisopropylamine or a mixture thereof, particularly preferably N-ethyldiisopropylamine. Work-up can be carried out analogously to process A.

For their part, the compounds of the formula VII can be prepared by deprotecting corresponding protected amino acid compounds IX analogously to known processes, for example according to Glenn L. Stahl et al., J. Org. Chem. 43(11), (1978), 2285-6 or A. K. Ghosh et al., Org. Lett. 3(4), (2001), 635-638. The preparation of VII from a Boc-protected amino acid compound IX is shown in the scheme below. Instead of the Boc group, it is also possible to employ other amino protective groups.

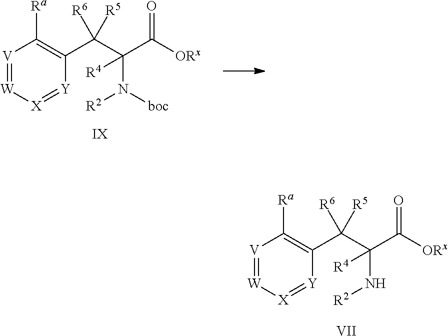

The conversion of a compound of the formula IX into the compound VII is typically carried out in the presence of an acid at temperatures in a range of from −30° C. to the boiling point of the reaction mixture, preferably from 0° C. to 50° C., especially preferably from 20° C. to 35° C. The reaction can be carried out in a solvent, preferably in an inert organic solvent.

Suitable solvents are the solvents quoted for the basic cyclization, in particular tetrahydrofuran or dichloromethane or mixtures thereof, preferably dichloromethane.

Suitable acids and acidic catalysts are, in principle, both Brönstedt and Lewis acids, in particular those mentioned above.

In one embodiment of the process according to the invention, the reaction is carried out in the presence of organic acids, for example in the presence of strong organic acids, such as formic acid, acetic acid or trifluoroacetic acid or mixtures thereof, preferably in the presence of trifluoroacetic acid. Work-up can be carried out analogously to process A.

The compounds of the formula IX can be prepared according to the reaction shown in the scheme below. The reaction of the compound V with the protected amino acid compound X can be carried out analogously to processes known from the literature, for example according to I. Ojima et al., J. Am. Chem. Soc., 109(21), (1987), 6537-6538 or J. M. McIntosh et al., Tetrahedron 48(30), (1992), 6219-6224.

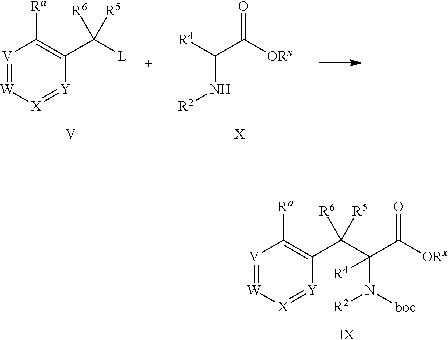

In this scheme, the variables have the meanings mentioned above. L is a leaving group, for example one of the leaving groups mentioned for process F. Instead of Boc, it is also possible to use other amino protective groups.

The reaction of V with X is generally carried out in the presence of a base. Suitable bases are the compounds quoted for process A. In a further preferred embodiment, the base used is lithium diisopropylamide, particularly preferably in an essentially equimolar amount, in particular in an equimolar amount.

The reaction is usually carried out at temperatures in the range of from −78° C. to the boiling point of the reaction mixture, preferably from −78° C. to the boiling point, especially preferably from −78° C. to 30° C.

The reaction can be carried out in a solvent, preferably in an inert organic solvent. Suitable solvents are, in principle, the solvents mentioned for the basic cyclization, in particular dichloromethane or tetrahydrofuran or mixtures thereof, preferably tetrahydrofuran. Work-up can be carried out analogously to process A.

Some of the compounds of the formula V are commercially available or can be prepared by transformations, described in the literature, of the corresponding commercially available precursors.

Some of the amino acid derivatives of the formula VIII, X or the derivative XV described below are likewise commercially available or can be prepared by transformations described in the literature of the corresponding commercially available precursors.

The compounds of the formula IV where R¹≠hydrogen can be prepared by reacting a piperazine compound of the formula IV in which R¹ is hydrogen with an alkylating agent or acylating agent which contains the radical R¹ different from hydrogen. In an analogous manner, compounds IV where R² hydrogen can be prepared by reacting a piperazine compound of the formula IV in which R² is hydrogen with an alkylating agent or acylating agent which contains the radical R² different from hydrogen. Such reactions can be carried out analogously to known processes, for example by the methods described by I. O. Donkor et al., Bioorg. Med. Chem. Lett. 11 (19) (2001), 2647-2649, B. B. Snider et al., Tetrahedron 57 (16) (2001), 3301-3307, I. Yasuhiro et al., J. Am. Chem. Soc. 124(47) (2002), 14017-14019 or M. Falorni et al., Europ. J. Org. Chem. (8) (2000), 1669-1675.

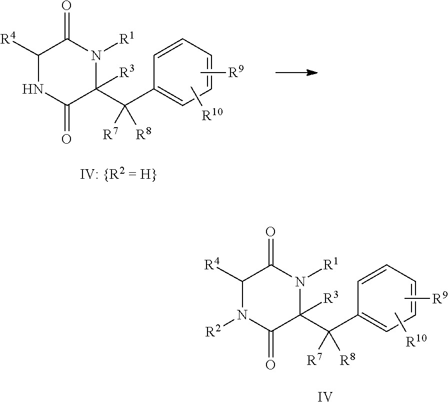

With respect to the alkylating agents or acylating agents, what was said for processes B and C applies in the same manner. With respect to the reaction conditions of these reactions, what was said above for processes B and C also applies.

The compounds of the formula IV can also be prepared by intramolecular cyclization of compounds of the formula XIII analogously to further known processes, for example according to T. Kawasaki et al., Org. Lett. 2(19) (2000), 3027-3029.

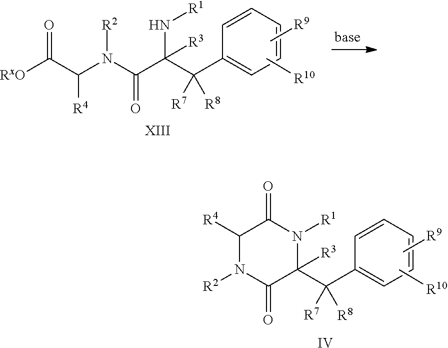

Here, OR^{x} is a suitable leaving group, R^{x} is here, for example, C₁-C₆-alkyl, in particular methyl, ethyl or benzyl.

In the formula XIII, the variables have the meaning given for formula II. The group OR^{x} is a suitable leaving group which is attached via oxygen. R^{x} is here, for example, C₁-C₆-alkyl, in particular methyl, ethyl, or phenyl-C₁-C₆-alkyl, for example benzyl.

The cyclization of the compounds of the formula XIII can be carried out in the presence of a base. In this case, the reaction is generally carried out at temperatures in the range of from 0° C. to the boiling point of the reaction mixture, preferably from 10° C. to 50° C., especially preferably from 15° C. to 35° C. The reaction can be carried out in a solvent, preferably in an inert organic solvent.

Suitable solvents are, in principle, the compounds quoted for the thermal cyclization, in particular a tetrahydrofuran/water mixture having a mixing ratio of from 1:10 to 10:1.

Suitable bases are the bases mentioned for the basic cyclization according to process A, in particular potassium tert-butoxide, 2-hydroxypyridine or an aqueous solution of ammonia or a mixture of these bases. Preferably, only one of these bases is used. In a particularly preferred embodiment, the reaction is carried out in the presence of an aqueous solution of ammonia which, for example, may be from 10 to 50% strength (v/v).

For their part, the compounds of the formula XIII can be prepared by the synthesis shown in the scheme below, analogously to known processes, for example according to Wilford L. Mendelson et al., Int. J. Peptide & Protein Research 35(3), (1990), 249-57, Glenn L. Stahl et al., J. Org. Chem. 43(11), (1978), 2285-6. or A. K. Ghosh et al., Org. Lett. 3(4), (2001), 635-638.

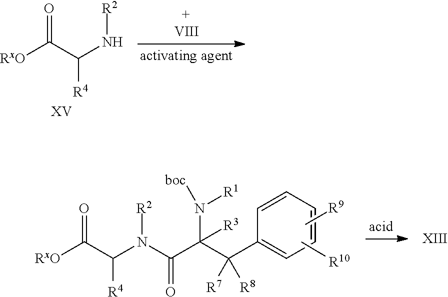

In the scheme, the variables R^{x}, R¹-R⁴ and R⁷-R¹⁰ have the meanings given for formula II or XIII. In a first step, the synthesis comprises the coupling of amino acid compounds XV with Boc-protected amino acids VIII in the presence of an activating agent.

The reaction of a compound of the formula XV with a compound of the formula VIII is usually carried out at temperatures in the range of from −30° C. to the boiling point of the reaction mixture, preferably from 0° C. to 50° C., especially preferably from 20° C. to 35° C. The reaction can be carried out in a solvent, preferably in an inert organic solvent. For further details, reference is made to the preparation of the compound VI by amidation of the amino acid compound VIII with the compound VII.

In general, the reaction requires the presence of an activating agent. Suitable activating agents are condensing agents, such as, for example, polystyrene-supported or non-polystyrene-supported dicyclohexylcarbodiimide (DCC), diisopropylcarbodiimide, 1-ethyl-3-(dimethylaminopropyl)carbodiimide (EDAC), carbonyldiimidazole, chloroformic esters, such as methyl chloroformate, ethyl chloroformate, isopropyl chloroformate, isobutyl chloroformate, sec-butyl chloroformate or allyl chloroformate, pivaloyl chloride, polyphosphoric acid, propanephosphonic anhydride, bis(2-oxo-3-oxazolidinyl)phosphoryl chloride (BOPCI) or sulfonyl chlorides, such as methanesulfonyl chloride, toluenesulfonyl chloride or benzenesulfonyl chloride. According to one embodiment, the preferred activating agents are EDAC and DCC.

The reaction of XV with VIII is preferably carried out in the presence of a base. Suitable bases are the bases quoted for process A. In one embodiment, the base used is triethylamine or N-ethyldiisopropylamine or mixtures thereof, particularly preferably N-ethyldiisopropylamine. Work-up can be carried out analogously to process A.

The deprotection of the compound XIV to give the compound XIII is typically carried out by treatment with an acid. The reaction is usually carried out at temperatures in the range of from −30° C. to the boiling point of the reaction mixture, preferably from 0° C. to 50° C., especially preferably from 20° C. to 35° C. The reaction can be carried out in a solvent, preferably in an inert organic solvent.

Suitable solvents are, in principle, the solvents mentioned for process A in connection with the basic cyclization, in particular tetrahydrofuran or dichloromethane or mixtures thereof, preferably dichloromethane.

The acids used are the acids mentioned for process A. For further details, reference is also made to the deprotection of VI to give the compound II. The reaction conditions mentioned there are also suitable for deprotecting the compound XIV. In one embodiment of the process according to the invention, the reaction is carried out in the presence of organic acids, in particular strong organic acids, for example in the presence of formic acid, acetic acid or trifluoroacetic acid or mixtures thereof, preferably in the presence of trifluoroacetic acid. Work-up can be carried out analogously to process A.

The compounds of the formula I in which R⁴ and R⁵ together are a covalent bond (formula I.A),

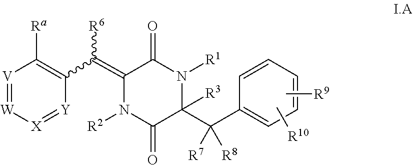

can be prepared by various routes using standard methods for the synthesis of organic compounds, preferably by the syntheses shown below:

Process E

Compounds of the formula XIV in which R⁶ is H (formula XIVa) can also be prepared by coupling the aldehyde Va with the piperazine IV in an aldol reaction, as illustrated in the scheme below:

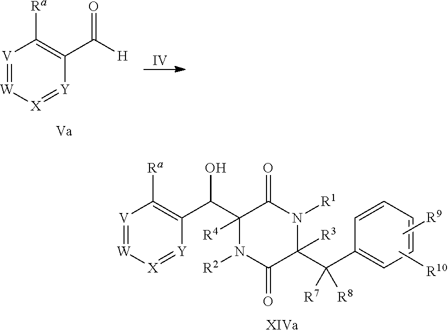

In the formulae, the variables have the meaning given for formula I. In formula XIVa, the groups R¹ and R² may independently of each other also be alkylcarbonyl, e.g. acetyl. The reaction is generally carried out analogously to the conditions described for the conversion of IIa into XIV.

The aldol reaction may also yield the corresponding aldol condensation product, i.e. compounds of the formula I.A in which R⁶ is H, directly. This is the case in particular when the reaction is carried out at elevated temperatures and with relatively long reaction times.

The aldehyde Va is either commercially available or can be synthesized according to known processes for preparing aldehydes. Such aldol condensations can be carried out analogously to the processes described in J. Org. Chem. 2000, 65 (24), 8402-8405.

In principle, the aldol reaction or condensation can also be used for preparing compounds I in which R⁶ does not have to be hydrogen but may also be C₁-C₆-alkyl, C₃-C₆-cycloalkyl, C₂-C₆-alkenyl, C₃-C₆-cycloalkenyl, C₂-C₆-alkynyl, C₃-C₆-cycloalkynyl, phenyl, phenyl-C₁-C₆-alkyl, heterocyclyl, heterocyclyl-C₁-C₆-alkyl; phenyl-[C₁-C₆-alkoxycarbonyl]-C₁-C₆-alkyl or phenylheterocyclyl-C₁-C₆-alkyl and preferably C₁-C₆-alkyl. In this case, instead of the aldehyde Va, the ketone Vb

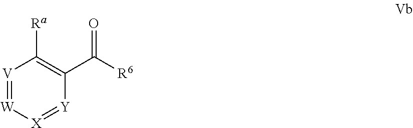

in which R⁶ is C₁-C₄-alkyl, C₁-C₄-haloalkyl, C₂-C₆-alkenyl, C₂-C₆-alkynyl, C₁-C₄-alkoxy, C₁-C₄-haloalkoxy, C₃-C₆-cycloalkyl, C₃-C₆-cycloalkenyl or C₃-C₆-cycloalkynyl and preferably C₁-C₆-alkyl is employed.

However, here, the formation of complex reaction mixtures is possible, in particular if R⁶ is a group in which the carbon atom bound in the a-position to the point of attachment carries a hydrogen atom. Furthermore, in most cases, more drastic reaction conditions are required, too, so that the aldolization is preferably only used for preparing compounds I.A in which R⁶ is H. Work-up can be carried out analogously to process A.

Process F

The process B is advantageously suitable for preparing compounds I.A where R¹=hydrogen. The conditions and preferences mentioned for process A also apply analogously to the preparation of the compounds I.A.

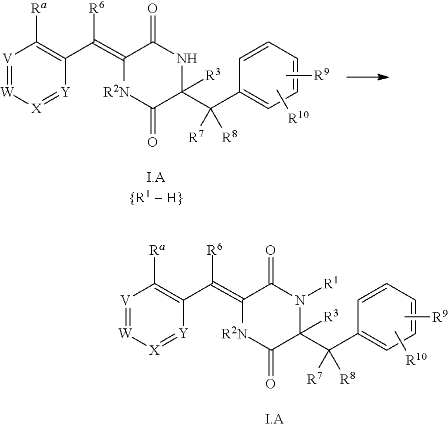

Solvents which are advantageously suitable are those listed for process A, inter alia toluene, dichloromethane, tetrahydrofuran or dimethylformamide or mixtures thereof, preferably tetrahydrofuran.

In a preferred embodiment, the compound I where R¹=H is reacted with the alkylating or acylating agent in the presence of a base. Suitable bases are the compounds listed for process A. The bases are generally employed in equimolar amounts. They can also be employed in excess or even as solvent. In a preferred embodiment, the base is added in an equimolar amount or in an essentially equimolar amount. In a further preferred embodiment, the base used is sodium hydride. Work-up can be carried out analogously to process A.

Alternatively, the alkylation or acylation of the group NR¹, and/or NR² in which R¹ and R², respectively, are hydrogen can also be carried out in the precursors. Thus, for example, compounds II, IV, VI, VII, VIII, IX, X, XIII, XIV, XV or XVI in which R¹ and/or R² are H can be N-alkylated or N-acylated as described above.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, the purification can also be carried out by recrystallization or digestion.

If individual compounds I cannot be obtained by the routes described above, they can be prepared by derivatization of other compounds I.

If the synthesis yields mixtures of isomers, a separation is generally however not necessarily required since in some cases the individual isomers can be interconverted during work-up for use or during application (for example under the action of light, acids or bases). Such conversions may also take place after application, for example in the case of the treatment of plants in the treated plant or in the harmful plant to be controlled.

The organic moieties mentioned for the substituents of the compounds according to the invention are collective terms for individual enumerations of the individual group members. All hydrocarbon chains, such as alkyl, haloalkyl, alkenyl, alkynyl, and the alkyl moieties and alkenyl moieties in alkoxy, haloalkoxy, alkylamino, dialkylamino, N-alkylsulfonylamino, alkenyloxy, alkynyloxy, alkoxyamino, alkylaminosulfonylamino, dialkylaminosulfonylamino, alkenylamino, alkynylamino, N-(alkenyl)-N-(alkyl)amino, N-(alkynyl)-N-(alkyl)amino, N-(alkoxy)-N-(alkyl)amino, N-(alkenyl)-N-(alkoxy)amino or N-(alkynyl)-N-(alkoxy)amino can be straight-chain or branched.

The prefix Cₙ-Cₘ- indicates the respective number of carbons of the hydrocarbon unit. Unless indicated otherwise, halogenated substituents preferably carry one to five identical or different halogen atoms, in particular fluorine atoms or chlorine atoms.

The meaning halogen denotes in each case fluorine, chlorine, bromine or iodine.

Examples of other meanings are:

alkyl and the alkyl moieties for example in alkoxy, alkylamino, dialkylamino, N-alkyl-sulfonylamino, alkylaminosulfonylamino, dialkylaminosulfonylamino, N-(alkenyl)-N-(alkyl)amino, N-(alkynyl)-N-(alkyl)amino, N-(alkoxy)-N-(alkyl)amino: saturated straight-chain or branched hydrocarbon radicals having one or more carbon atoms, for example 1 or 2, 1 to 4 or 1 to 6 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl. In one embodiment according to the invention, alkyl denotes small alkyl groups, such as C₁-C₄-alkyl. In another embodiment according to the invention, alkyl denotes relatively large alkyl groups, such as C₅-C₆-alkyl.

Haloalkyl: an alkyl radical as mentioned above, some or all of whose hydrogen atoms are substituted by halogen atoms, such as fluorine, chlorine, bromine and/or iodine, for example chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl and nonafluorobutyl.

Cycloalkyl and the cycloalkyl moieties for example in cycloalkoxy or cycloalkylcarbonyl: monocyclic saturated hydrocarbon groups having three or more carbon atoms, for example 3 to 6 carbon ring members, such as cyclopropyl, cyclobutyl, cyclopentyl and cyclohexyl.

Alkenyl and the alkenyl moieties for example in alkenylamino, alkenyloxy, N-(alkenyl)-N-(alkyl)amino, N-(alkenyl)-N-(alkoxy)amino: monounsaturated straight-chain or branched hydrocarbon radicals having two or more carbon atoms, for example 2 to 4, 2 to 6 or 3 to 6 carbon atoms, and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl.

Cycloalkenyl: monocyclic monounsaturated hydrocarbon groups having 3 to 6, preferably 5 or 6, carbon ring members, such as cyclopenten-1-yl, cyclopenten-3-yl, cyclohexen-1-yl, cyclohexen-3-yl, cyclohexen-4-yl.

Alkynyl and the alkynyl moieties for example in alkynyloxy, alkynylamino, N-(alkynyl)-N-(alkyl)amino or N-(alkynyl)-N-(alkoxy)amino: straight-chain or branched hydrocarbon groups having two or more carbon atoms, for example 2 to 4, 2 to 6 or 3 to 6 carbon atoms, and a triple bond in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl.

Alkoxy: alkyl as defined above which is attached via an oxygen atom, for example methoxy, ethoxy, n-propoxy, 1-methylethoxy, butoxy, 1-methylpropoxy, 2-methylpropoxy or 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexyloxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy.

A 5- or 6-membered heterocycle: a cyclic group which has 5 or 6 ring atoms, 1, 2, 3 or 4 ring atoms being heteroatoms selected from the group consisting of O, S and N, where the cyclic group is saturated, partially unsaturated or aromatic. Examples of heterocyclic groups are:

5-membered saturated rings which are attached via carbon, such as

tetrahydrofuran-2-yl, tetrahydrofuran-3-yl, tetrahydrothien-2-yl, tetrahydrothien-3-yl, tetrahydropyrrol-2-yl, tetrahydropyrrol-3-yl, tetrahydropyrazol-3-yl, tetrahydropyrazol-4-yl, tetrahydroisoxazol-3-yl, tetrahydroisoxazol-4-yl, tetrahydroisoxazol-5-yl, 1,2-oxathiolan-3-yl, 1,2-oxathiolan-4-yl, 1,2-oxathiolan-5-yl, tetrahydroisothiazol-3-yl, tetrahydroisothiazol-4-yl, tetrahydroisothiazol-5-yl, 1,2-dithiolan-3-yl, 1,2-dithiolan-4-yl, tetrahydroimidazol-2-yl, tetrahydroimidazol-4-yl, tetrahydrooxazol-2-yl, tetrahydrooxazol-4-yl, tetrahydrooxazol-5-yl, tetrahydrothiazol-2-yl, tetrahydrothiazol-4-yl, tetrahydrothiazol-5-yl, 1,3-dioxolan-2-yl, 1,3-dioxolan-4-yl, 1,3-oxathiolan-2-yl, 1,3-oxathiolan-4-yl, 1,3-oxathiolan-5-yl, 1,3-dithiolan-2-yl, 1,3-dithiolan-4-yl, 1,3,2-dioxathiolan-4-yl;

6-membered saturated rings which are attached via carbon, such as:

tetrahydropyran-2-yl, tetrahydropyran-3-yl, tetrahydropyran-4-yl, piperidin-2-yl, piperidin-3-yl, piperidin-4-yl, tetrahydrothiopyran-2-yl, tetrahydrothiopyran-3-yl, tetrahydrothiopyran-4-yl, 1,3-dioxan-2-yl, 1,3-dioxan-4-yl, 1,3-dioxan-5-yl, 1,4-dioxan-2-yl, 1,3-dithian-2-yl, 1,3-dithian-4-yl, 1,3-dithian-5-yl, 1,4-dithian-2-yl, 1,3-oxathian-2-yl, 1,3-oxathian-4-yl, 1,3-oxathian-5-yl, 1,3-oxathian-6-yl, 1,4-oxathian-2-yl, 1,4-oxathian-3-yl, 1,2-dithian-3-yl, 1,2-dithian-4-yl, hexahydropyrimidin-2-yl, hexahydropyrimidin-4-yl, hexahydropyrimidin-5-yl, hexahydropyrazin-2-yl, hexahydropyridazin-3-yl, hexahydropyridazin-4-yl, tetrahydro-1,3-oxazin-2-yl, tetrahydro-1,3-oxazin-4-yl, tetrahydro-1,3-oxazin-5-yl, tetrahydro-1,3-oxazin-6-yl, tetrahydro-1,3-thiazin-2-yl, tetrahydro-1,3-thiazin-4-yl, tetrahydro-1,3-thiazin-5-yl, tetrahydro-1,3-thiazin-6-yl, tetrahydro-1,4-thiazin-2-yl, tetrahydro-1,4-thiazin-3-yl, tetrahydro-1,4-oxazin-2-yl, tetrahydro-1,4-oxazin-3-yl, tetrahydro-1,2-oxazin-3-yl, tetrahydro-1,2-oxazin-4-yl, tetrahydro-1,2-oxazin-5-yl, tetrahydro-1,2-oxazin-6-yl;

5-membered saturated rings which are attached via nitrogen, such as:

tetrahydropyrrol-1-yl, tetrahydropyrazol-1-yl, tetrahydroisoxazol-2-yl, tetrahydroisothiazol-2-yl, tetrahydroimidazol-1-yl, tetrahydrooxazol-3-yl, tetrahydrothiazol-3-yl;

6-membered saturated rings which are attached via nitrogen, such as:

piperidin-1-yl, hexahydropyrimidin-1-yl, hexahydropyrazin-1-yl, hexahydropyridazin-1-yl, tetrahydro-1,3-oxazin-3-yl, tetrahydro-1,3-thiazin-3-yl, tetrahydro-1,4-thiazin-4-yl, tetrahydro-1,4-oxazin-4-yl, tetrahydro-1,2-oxazin-2-yl;

5-membered partially unsaturated rings which are attached via carbon, such as:

2,3-dihydrofuran-2-yl, 2,3-dihydrofuran-3-yl, 2,5-dihydrofuran-2-yl, 2,5-dihydrofuran-3-yl, 4,5-dihydrofuran-2-yl, 4,5-dihydrofuran-3-yl, 2,3-dihydrothien-2-yl, 2,3-dihydrothien-3-yl, 2,5-dihydrothien-2-yl, 2,5-dihydrothien-3-yl, 4,5-dihydrothien-2-yl, 4,5-dihydrothien-3-yl, 2,3-dihydro-1H-pyrrol-2-yl, 2,3-dihydro-1H-pyrrol-3-yl, 2,5-dihydro-1H-pyrrol-2-yl, 2,5-dihydro-1H-pyrrol-3-yl, 4,5-dihydro-1H-pyrrol-2-yl, 4,5-dihydro-1H-pyrrol-3-yl, 3,4-dihydro-2H-pyrrol-2-yl, 3,4-dihydro-2H-pyrrol-3-yl, 3,4-dihydro-5H-pyrrol-2-yl, 3,4-dihydro-5H-pyrrol-3-yl, 4,5-dihydro-1H-pyrazol-3-yl, 4,5-dihydro-1H-pyrazol-4-yl, 4,5-dihydro-1H-pyrazol-5-yl, 2,5-dihydro-1H-pyrazol-3-yl, 2,5-dihydro-1H-pyrazol-4-yl, 2,5-dihydro-1H-pyrazol-5-yl, 4,5-dihydroisoxazol-3-yl, 4,5-dihydroisoxazol-4-yl, 4,5-dihydroisoxazol-5-yl, 2,5-dihydroisoxazol-3-yl, 2,5-dihydroisoxazol-4-yl, 2,5-dihydroisoxazol-5-yl, 2,3-dihydroisoxazol-3-yl, 2,3-dihydroisoxazol-4-yl, 2,3-dihydroisoxazol-5-yl, 4,5-dihydroisothiazol-3-yl, 4,5-dihydroisothiazol-4-yl, 4,5-dihydroisothiazol-5-yl, 2,5-dihydroisothiazol-3-yl, 2,5-dihydroisothiazol-4-yl, 2,5-dihydroisothiazol-5-yl, 2,3-dihydroisothiazol-3-yl, 2,3-dihydroisothiazol-4-yl, 2,3-dihydroisothiazol-5-yl, Δ ³-1,2-dithiol-3-yl, Δ ³-1,2-dithiol-4-yl, Δ ³-1,2-dithiol-5-yl, 4,5-dihydro-1H-imidazol-2-yl, 4,5-dihydro-1H-imidazol-4-yl, 4,5-dihydro-1H-imidazol-5-yl, 2,5-dihydro-1H-imidazol-2-yl, 2,5-dihydro-1H-imidazol-4-yl, 2,5-dihydro-1H-imidazol-5-yl, 2,3-dihydro-1H-imidazol-2-yl, 2,3-dihydro-1H-imidazol-4-yl, 4,5-dihydrooxazol-2-yl, 4,5-dihydrooxazol-4-yl, 4,5-dihydrooxazol-5-yl, 2,5-dihydrooxazol-2-yl, 2,5-dihydrooxazol-4-yl, 2,5-dihydrooxazol-5-yl, 2,3-dihydrooxazol-2-yl, 2,3-dihydrooxazol-4-yl, 2,3-dihydrooxazol-5-yl, 4,5-dihydrothiazol-2-yl, 4,5-dihydrothiazol-4-yl, 4,5-dihydrothiazol-5-yl, 2,5-dihydrothiazol-2-yl, 2,5-dihydrothiazol-4-yl, 2,5-dihydrothiazol-5-yl, 2,3-dihydrothiazol-2-yl, 2,3-dihydrothiazol-4-yl, 2,3-dihydrothiazol-5-yl, 1,3-dioxol-2-yl, 1,3-dioxol-4-yl, 1,3-dithiol-2-yl, 1,3-dithiol-4-yl, 1,3-oxathiol-2-yl, 1,3-oxathiol-4-yl, 1,3-oxathiol-5-yl;

6-membered partially unsaturated rings which are attached via carbon, such as:

2H-3,4-dihydropyran-6-yl, 2H-3,4-dihydropyran-5-yl, 2H-3,4-dihydropyran-4-yl, 2H-3,4-dihydropyran-3-yl, 2H-3,4-dihydropyran-2-yl, 2H-3,4-dihydrothiopyran-6-yl, 2H-3,4-dihydrothiopyran-5-yl, 2H-3,4-dihydrothiopyran-4-yl, 2H-3,4-dihydrothiopyran-3-yl, 2H-3,4-dihydrothiopyran-2-yl, 1,2,3,4-tetrahydropyridin-6-yl, 1,2,3,4-tetrahydropyridin-5-yl, 1,2,3,4-tetrahydropyridin-4-yl, 1,2,3,4-tetrahydropyridin-3-yl, 1,2,3,4-tetrahydropyridin-2-yl, 2H-5,6-dihydropyran-2-yl, 2H-5,6-dihydropyran-3-yl, 2H-5,6-dihydropyran-4-yl, 2H-5,6-dihydropyran-5-yl, 2H-5,6-dihydropyran-6-yl, 2H-5,6-dihydrothiopyran-2-yl, 2H-5,6-dihydrothiopyran-3-yl, 2H-5,6-dihydrothiopyran-4-yl, 2H-5,6-dihydrothiopyran-5-yl, 2H-5,6-dihydrothiopyran-6-yl, 1,2,5,6-tetrahydropyridin-2-yl, 1,2,5,6-tetrahydropyridin-3-yl, 1,2,5,6-tetrahydropyridin-4-yl, 1,2,5,6-tetrahydropyridin-5-yl, 1,2,5,6-tetrahydropyridin-6-yl, 2,3,4,5-tetrahydropyridin-2-yl, 2,3,4,5-tetrahydropyridin-3-yl, 2,3,4,5-tetrahydropyridin-4-yl, 2,3,4,5-tetrahydropyridin-5-yl, 2,3,4,5-tetrahydropyridin-6-yl, 4H-pyran-2-yl, 4H-pyran-3-yl-, 4H-pyran-4-yl, 4H-thiopyran-2-yl, 4H-thiopyran-3-yl, 4H-thiopyran-4-yl, 1,4-dihydropyridin-2-yl, 1,4-dihydropyridin-3-yl, 1,4-dihydropyridin-4-yl, 2H-pyran-2-yl, 2H-pyran-3-yl, 2H-pyran-4-yl, 2H-pyran-5-yl, 2H-pyran-6-yl, 2H-thiopyran-2-yl, 2H-thiopyran-3-yl, 2H-thiopyran-4-yl, 2H-thiopyran-5-yl, 2H-thiopyran-6-yl, 1,2-dihydropyridin-2-yl, 1,2-dihydropyridin-3-yl, 1,2-dihydropyridin-4-yl, 1,2-dihydropyridin-5-yl, 1,2-dihydropyridin-6-yl, 3,4-dihydropyridin-2-yl, 3,4-dihydropyridin-3-yl, 3,4-dihydropyridin-4-yl, 3,4-dihydropyridin-5-yl, 3,4-dihydropyridin-6-yl, 2,5-dihydropyridin-2-yl, 2,5-dihydropyridin-3-yl, 2,5-dihydropyridin-4-yl, 2,5-dihydropyridin-5-yl, 2,5-dihydropyridin-6-yl, 2,3-dihydropyridin-2-yl, 2,3-dihydropyridin-3-yl, 2,3-dihydropyridin-4-yl, 2,3-dihydropyridin-5-yl, 2,3-dihydropyridin-6-yl, 2H-5,6-dihydro-1,2-oxazin-3-yl, 2H-5,6-dihydro-1,2-oxazin-4-yl, 2H-5,6-dihydro-1,2-oxazin-5-yl, 2H-5,6-dihydro-1,2-oxazin-6-yl, 2H-5,6-dihydro-1,2-thiazin-3-yl, 2H-5,6-dihydro-1,2-thiazin-4-yl, 2H-5,6-dihydro-1,2-thiazin-5-yl, 2H-5,6-dihydro-1,2-thiazin-6-yl, 4H-5,6-dihydro-1,2-oxazin-3-yl, 4H-5,6-dihydro-1,2-oxazin-4-yl, 4H-5,6-dihydro-1,2-oxazin-5-yl, 4H-5,6-dihydro-1,2-oxazin-6-yl, 4H-5,6-dihydro-1,2-thiazin-3-yl, 4H-5,6-dihydro-1,2-thiazin-4-yl, 4H-5,6-dihydro-1,2-thiazin-5-yl, 4H-5,6-dihydro-1,2-thiazin-6-yl, 2H-3,6-dihydro-1,2-oxazin-3-yl, 2H-3,6-dihydro-1,2-oxazin-4-yl, 2H-3,6-dihydro-1,2-oxazin-5-yl, 2H-3,6-dihydro-1,2-oxazin-6-yl, 2H-3,6-dihydro-1,2-thiazin-3-yl, 2H-3,6-dihydro-1,2-thiazin-4-yl, 2H-3,6-dihydro-1,2-thiazin-5-yl, 2H-3,6-dihydro-1,2-thiazin-6-yl, 2H-3,4-dihydro-1,2-oxazin-3-yl, 2H-3,4-dihydro-1,2-oxazin-4-yl, 2H-3,4-dihydro-1,2-oxazin-5-yl, 2H-3,4-dihydro-1,2-oxazin-6-yl, 2H-3,4-dihydro-1,2-thiazin-3-yl, 2H-3,4-dihydro-1,2-thiazin-4-yl, 2H-3,4-dihydro-1,2-thiazin-5-yl, 2H-3,4-dihydro-1,2-thiazin-6-yl, 2,3,4,5-tetrahydropyridazin-3-yl, 2,3,4,5-tetrahydropyridazin-4-yl, 2,3,4,5-tetrahydropyridazin-5-yl, 2,3,4,5-tetrahydropyridazin-6-yl, 3,4,5,6-tetrahydropyridazin-3-yl, 3,4,5,6-tetrahydropyridazin-4-yl, 1,2,5,6-tetrahydropyridazin-3-yl, 1,2,5,6-tetrahydropyridazin-4-yl, 1,2,5,6-tetrahydropyridazin-5-yl, 1,2,5,6-tetrahydropyridazin-6-yl, 1,2,3,6-tetrahydropyridazin-3-yl, 1,2,3,6-tetrahydropyridazin-4-yl, 4H-5,6-dihydro-1,3-oxazin-2-yl, 4H-5,6-dihydro-1,3-oxazin-4-yl, 4H-5,6-dihydro-1,3-oxazin-5-yl, 4H-5,6-dihydro-1,3-oxazin-6-yl, 4H-5,6-dihydro-1,3-thiazin-2-yl, 4H-5,6-dihydro-1,3-thiazin-4-yl, 4H-5,6-dihydro-1,3-thiazin-5-yl, 4H-5,6-dihydro-1,3-thiazin-6-yl, 3,4,5,6-tetrahydropyrimidin-2-yl, 3,4,5,6-tetrahydropyrimidin-4-yl, 3,4,5,6-tetrahydropyrimidin-5-yl, 3,4,5,6-tetrahydropyrimidin-6-yl, 1,2,3,4-tetrahydropyrazin-2-yl, 1,2,3,4-tetrahydropyrazin-5-yl, 1,2,3,4-tetrahydropyrimidin-2-yl, 1,2,3,4-tetrahydropyrimidin-4-yl, 1,2,3,4-tetrahydropyrimidin-5-yl, 1,2,3,4-tetrahydropyrimidin-6-yl, 2,3-dihydro-1,4-thiazin-2-yl, 2,3-dihydro-1,4-thiazin-3-yl, 2,3-dihydro-1,4-thiazin-5-yl, 2,3-dihydro-1,4-thiazin-6-yl, 2H-1,2-oxazin-3-yl, 2H-1,2-oxazin-4-yl, 2H-1,2-oxazin-5-yl, 2H-1,2-oxazin-6-yl, 2H-1,2-thiazin-3-yl, 2H-1,2-thiazin-4-yl, 2H-1,2-thiazin-5-yl, 2H-1,2-thiazin-6-yl, 4H-1,2-oxazin-3-yl, 4H-1,2-oxazin-4-yl, 4H-1,2-oxazin-5-yl, 4H-1,2-oxazin-6-yl, 4H-1,2-thiazin-3-yl, 4H-1,2-thiazin-4-yl, 4H-1,2-thiazin-5-yl, 4H-1,2-thiazin-6-yl, 6H-1,2-oxazin-3-yl, 6H-1,2-oxazin-4-yl, 6H-1,2-oxazin-5-yl, 6H-1,2-oxazin-6-yl, 6H-1,2-thiazin-3-yl, 6H-1,2-thiazin-4-yl, 6H-1,2-thiazin-5-yl, 6H-1,2-thiazin-6-yl, 2H-1,3-oxazin-2-yl, 2H-1,3-oxazin-4-yl, 2H-1,3-oxazin-5-yl, 2H-1,3-oxazin-6-yl, 2H-1,3-thiazin-2-yl, 2H-1,3-thiazin-4-yl, 2H-1,3-thiazin-5-yl, 2H-1,3-thiazin-6-yl, 4H-1,3-oxazin-2-yl, 4H-1,3-oxazin-4-yl, 4H-1,3-oxazin-5-yl, 4H-1,3-oxazin-6-yl, 4H-1,3-thiazin-2-yl, 4H-1,3-thiazin-4-yl, 4H-1,3-thiazin-5-yl, 4H-1,3-thiazin-6-yl, 6H-1,3-oxazin-2-yl, 6H-1,3-oxazin-4-yl, 6H-1,3-oxazin-5-yl, 6H-1,3-oxazin-6-yl, 6H-1,3-thiazin-2-yl, 6H-1,3-oxazin-4-yl, 6H-1,3-oxazin-5-yl, 6H-1,3-thiazin-6-yl, 2H-1,4-oxazin-2-yl, 2H-1,4-oxazin-3-yl, 2H-1,4-oxazin-5-yl, 2H-1,4-oxazin-6-yl, 2H-1,4-thiazin-2-yl, 2H-1,4-thiazin-3-yl, 2H-1,4-thiazin-5-yl, 2H-1,4-thiazin-6-yl, 4H-1,4-oxazin-2-yl, 4H-1,4-oxazin-3-yl, 4H-1,4-thiazin-2-yl, 4H-1,4-thiazin-3-yl, 1,4-dihydropyridazin-3-yl, 1,4-dihydropyridazin-4-yl, 1,4-dihydropyridazin-5-yl, 1,4-dihydropyridazin-6-yl, 1,4-dihydropyrazin-2-yl, 1,2-dihydropyrazin-2-yl, 1,2-dihydropyrazin-3-yl, 1,2-dihydropyrazin-5-yl, 1,2-dihydropyrazin-6-yl, 1,4-dihydropyrimidin-2-yl, 1,4-dihydropyrimidin-4-yl, 1,4-dihydropyrimidin-5-yl, 1,4-dihydropyrimidin-6-yl, 3,4-dihydropyrimidin-2-yl, 3,4-dihydropyrimidin-4-yl, 3,4-dihydropyrimidin-5-yl or 3,4-dihydropyrimidin-6-yl;

5-membered partially unsaturated rings which are attached via nitrogen, such as:

2,3-dihydro-1H-pyrrol-1-yl, 2,5-dihydro-1H-pyrrol-1-yl, 4,5-dihydro-1H-pyrazol-1-yl, 2,5-dihydro-1H-pyrazol-1-yl, 2,3-dihydro-1H-pyrazol-1-yl, 2,5-dihydroisoxazol-2-yl, 2,3-dihydroisoxazol-2-yl, 2,5-dihydroisothiazol-2-yl, 2,3-dihydroisoxazol-2-yl, 4,5-dihydro-1H-imidazol-1-yl, 2,5-dihydro-1H-imidazol-1-yl, 2,3-dihydro-1H-imidazol-1-yl, 2,3-dihydrooxazol-3-yl, 2,3-dihydrothiazol-3-yl, 1,2,4-Δ⁴-oxadiazolin-2-yl, 1,2,4-Δ²-oxadiazolin-4-yl, 1,2,4-Δ³-oxadiazolin-2-yl, 1,3,4-Δ²-oxadiazolin-4-yl, 1,2,4-Δ⁵-thiadiazolin-2-yl, 1,2,4-Δ³-thiadiazolin-2-yl, 1,2,4-Δ²-thiadiazolin-4-yl, 1,3,4-Δ²-thiadiazolin-4-yl, 1,2,3-Δ²-triazolin-1-yl, 1,2,4-Δ²-triazolin-1-yl, 1,2,4-Δ²-triazolin-4-yl, 1,2,4-Δ³-triazolin-1-yl, 1,2,4-Δ¹-triazolin-4-yl;

6-membered partially unsaturated rings which are attached via nitrogen, such as:

1,2,3,4-tetrahydropyridin-1-yl, 1,2,5,6-tetrahydropyridin-1-yl, 1,4-dihydropyridin-1-yl, 1,2-dihydropyridin-1-yl, 2H-5,6-dihydro-1,2-oxazin-2-yl, 2H-5,6-dihydro-1,2-thiazin-2-yl, 2H-3,6-dihydro-1,2-oxazin-2-yl, 2H-3,6-dihydro-1,2-thiazin-2-yl, 2H-3,4-dihydro-1,2-oxazin-2-yl, 2H-3,4-dihydro-1,2-thiazin-2-yl, 2,3,4,5-tetrahydropyridazin-2-yl, 1,2,5,6-tetrahydropyridazin-1-yl, 1,2,5,6-tetrahydropyridazin-2-yl, 1,2,3,6-tetrahydropyridazin-1-yl, 3,4,5,6-tetrahydropyrimidin-3-yl, 1,2,3,4-tetrahydropyrazin-1-yl, 1,2,3,4-tetrahydropyrimidin-1-yl, 1,2,3,4-tetrahydropyrimidin-3-yl, 2,3-dihydro-1,4-thiazin-4-yl, 2H-1,2-oxazin-2-yl, 2H-1,2-thiazin-2-yl, 4H-1,4-oxazin-4-yl, 4H-1,4-thiazin-4-yl, 1,4-dihydropyridazin-1-yl, 1,4-dihydropyrazin-1-yl, 1,2-dihydropyrazin-1-yl, 1,4-dihydropyrimidin-1-yl or 3,4-dihydropyrimidin-3-yl;

5-membered heteroaromatic rings which are attached via carbon, such as:

2-furyl, 3-furyl, 2-thienyl, 3-thienyl, pyrrol-2-yl, pyrrol-3-yl, pyrazol-3-yl, pyrazol-4-yl, isoxazol-3-yl, isoxazol-4-yl, isoxazol-5-yl, isothiazol-3-yl, isothiazol-4-yl, isothiazol-5-yl, imidazol-2-yl, imidazol-4-yl, oxazol-2-yl, oxazol-4-yl, oxazol-5-yl, thiazol-2-yl, thiazol-4-yl, thiazol-5-yl, 1,2,3-oxadiazol-4-yl, 1,2,3-oxadiazol-5-yl, 1,2,4-oxadiazol-3-yl, 1,2,4-oxadiazol-5-yl, 1,3,4-oxadiazol-2-yl, 1,2,3-thiadiazol-4-yl, 1,2,3-thiadiazol-5-yl, 1,2,4-thiadiazol-3-yl, 1,2,4-thiadiazol-5-yl, 1,3,4-thiadiazolyl-2-yl, 1,2,3-triazol-4-yl, 1,2,4-triazol-3-yl, [1H]-tetrazol-5-yl and [2H]-tetrazol-5-yl;

6-membered heteroaromatic rings which are attached via carbon, such as:

pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl, 1,3,5-triazin-2-yl, 1,2,4-triazin-3-yl, 1,2,4-triazin-5-yl and 1,2,4-triazin-6-yl;

5-membered heteroaromatic rings which are attached via nitrogen, such as:

pyrrol-1-yl, pyrazol-1-yl, imidazol-1-yl, 1,2,3-triazol-1-yl, 1,2,4-triazol-1-yl, [1H]-tetrazol-1-yl and [2H]-tetrazol-2-yl.

The heterocycles mentioned above can be substituted in the manner indicated. In the heterocycles mentioned above, a sulfur atom can be oxidized to S═O or S(═O)₂.

At the carbon atom which carries the group R³ and/or R⁴, the compounds of the formula I have a center of chirality. In addition, depending on the substitution pattern, they may contain one or more further centers of chirality. Accordingly, the compounds according to the invention can be present as pure enantiomers or diastereomers or as enantiomer or diastereomer mixtures. The invention provides both the pure enantiomers or diastereomers and their mixtures.

The compounds of the formula I may also be present in the form of their agriculturally useful salts, the type of salt generally not being important. Suitable salts are generally the salts of those cations or the acid addition salts of those acids whose cations and anions, respectively, have no adverse effect on the herbicidal activity of the compounds I.

Suitable cations are in particular ions of the alkali metals, preferably lithium, sodium or potassium, of the alkaline earth metals, preferably calcium or magnesium, and of the transition metals, preferably manganese, copper, zinc or iron. Another cation that may be used is ammonium, where, if desired, one to four hydrogen atoms may be replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, dimethylammonium, diisopropylammonium, tetramethylammonium, tetrabutylammonium, 2-(2-hydroxyeth-1-oxy)eth-1-ylammonium, di(2-hydroxyeth-1-yl)ammonium, trimethylbenzylammonium. Also suitable are phosphonium ions, sulfonium ions, preferably tri(C₁-C₄-alkyl)sulfonium, or sulfoxonium ions, preferably tri(C₁-C₄-alkyl)sulfoxonium. Anions of suitable acid addition salts are primarily chloride, bromide, fluoride, hydrogensulfate, sulfate, dihydrogenphosphate, hydrogenphosphate, nitrate, bicarbonate, carbonate, hexafluorosilicate, hexafluorophosphate, benzoate and also the anions of C₁-C₄-alkanoic acids, preferably formate, acetate, propionate or butyrate.

With respect to the variables, the particularly preferred embodiments of the intermediates correspond to those of the groups of the formula I.

In a particular embodiment, the variables of the compounds of the formula I have the following meanings, these meanings, both on their own and in combination with one another, being particular embodiments of the compounds of the formula I:

Of the variables V, W, X and Y, from zero to three groups are preferably nitrogen.

In a preferred embodiment the six-membered ring comprising the groups V, W, X, Y is a substituted heteroaromatic such as pyridine, pyrimidine and pyrazine, in particular pyridine.

A further particularly preferred embodiment relates to phenyl.

In one embodiment of the compounds of the formula I, V, W, X and Y are C—R^{b}. These compounds correspond to the formula I.1,

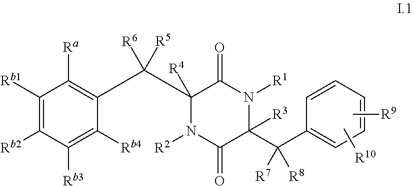

in which the groups R^{b1}, R^{b2}, R^{b3} and R^{b4} each correspond to a group R^{b} and preferably have the following meanings:

R^{b} is H, alkyl, alkoxy, in particular H, F, CH₃ or OCH₃;

R^{b2} is H, halogen, alkyl, halomethyl, in particular H, C₁, CF₃ or CH₃;

R^{b3} is halogen, CH₃ or OCH₃, in particular F, CH₃ or OCH₃;

R^{b4} is H, halogen, CH₃ or OCH₃, in particular H, F, Cl, Br, CH₃ or OCH₃.

In a further embodiment of the compounds of the formula I, V is N and W, X and Y are C—R^{b}. These compounds correspond to the formula I.2

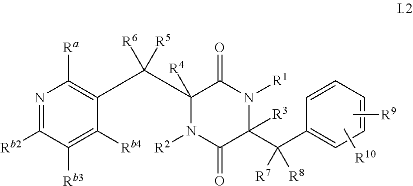

in which the groups R^{b2}, R^{b3} and R^{b4} each correspond to a group R^{b} and preferably have the following meanings:

R^{b2} is H, halogen, alkyl, halomethyl, in particular H, C₁, CF₃ or CH₃;

R^{b3} is halogen, CH₃ or OCH₃, in particular F, CH₃ or OCH₃;

R^{b4} is H, halogen, CH₃ or OCH₃, in particular H, F, Cl, Br, CH₃ or OCH₃.

In a further embodiment of the compounds of the formula I, V, X and Y are C—R^{b} and W is N. These compounds correspond to the formula I.3

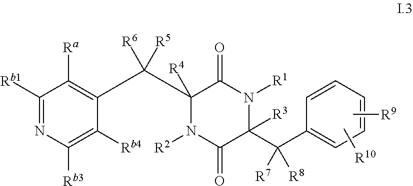

in which the groups R^{b1}, R^{b3} and R^{b4} each correspond to a group R^{b} and preferably have the following meanings:

R^{b1} is H;

R^{b3} is halogen, CH₃ or OCH₃, in particular F, CH₃ or OCH₃;

R^{b4} is H, halogen, CH₃ or OCH₃, in particular H, F, Cl, Br, CH₃ or OCH₃.

In a further embodiment of the compounds of the formula I, V, W and Y are C—R^{b} and X is N. These compounds correspond to the formula I.4

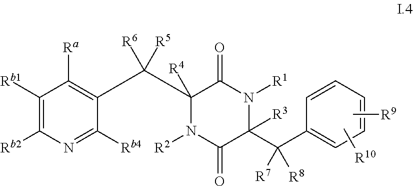

in which the groups R^{b1}, R^{b2} and R^{b4} each correspond to a group R^{b} and preferably have the following meanings:

R^{b1} is H;

R^{b2} is H, halogen, halomethyl, in particular H, C₁, CF₃;

R^{b4} is H, halogen, CH₃ or OCH₃, in particular H, F, Cl, Br, CH₃ or OCH₃.

In a further embodiment of the compounds of the formula I, V, W and X are C—R^{b} and Y is N. These compounds correspond to the formula I.5

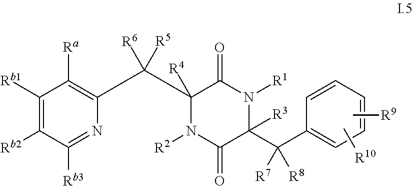

in which the groups R^{b1}, R^{b2} and R^{b3} each correspond to a group R^{b} and preferably have the following meanings:

R^{b1} is H,

R^{b2} is H, halogen, halomethyl, in particular H, C₁, CF₃;

R^{b3} is halogen, CH₃ or OCH₃, in particular F, CH₃ or OCH₃.

In a further embodiment of the compounds of the formula I, V and X are N and W and Y are C—R^{b}. These compounds correspond to the formula I.6

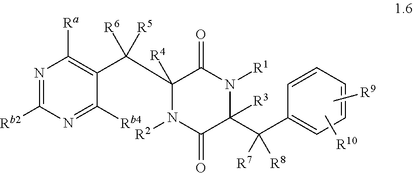

in which the groups R^{b2} and R^{b4} each correspond to a group R^{b} and preferably have the following meanings:

R^{b2} is H, halogen, halomethyl, in particular H, C₁, CF₃;

R^{b4} is H, halogen, CH₃ or OCH₃, in particular H, F, Cl, Br, CH₃ or OCH₃.

In a further embodiment of the compounds of the formula I, V and Y are N and W and X are C—R^{b}. These compounds correspond to the formula I.7

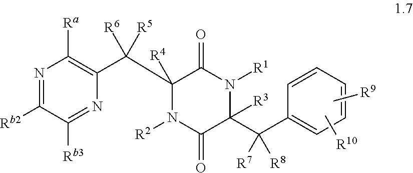

in which the groups R^{b2} and R^{b3} each correspond to a group R^{b} and preferably have the following meanings:

R^{b2} is H, halogen, halomethyl, in particular H, C₁, CF₃;

R^{b3} is halogen, CH₃ or OCH₃, in particular F, CH₃ or OCH₃.

In a further embodiment of the compounds of the formula I, V and W are N and X and Y are C—R^{b}. These compounds correspond to the formula I.8

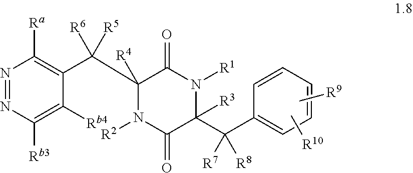

in which the groups R^{b3} and R^{b4} each correspond to a group R^{b} and preferably have the following meanings:

R^{b3} is halogen, CH₃ or OCH₃, in particular F, CH₃ or OCH₃;

R^{b4} is H, halogen, CH₃ or OCH₃, in particular H, F, Cl, Br, CH₃ or OCH₃.

In a further embodiment of the compounds of the formula I, W and Y are N and V and X are C—R^{b}. These compounds correspond to the formula I.9

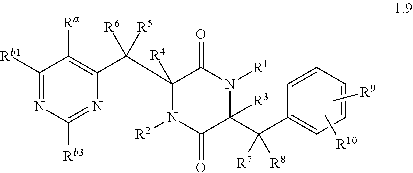

in which the groups R^{b1}, and R^{b3} each correspond to a group R^{b} and preferably have the following meanings:

R^{b1} is H_{,}

R^{b3} is halogen, CH₃ or OCH₃, in particular F, CH₃ or OCH₃.

In a further embodiment of the compounds of the formula I, W and X are N and V and Y are C—R^{b}. These compounds correspond to the formula I.10

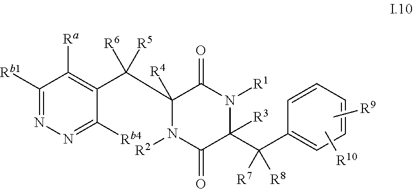

in which the groups R^{b1} and R^{b4} each correspond to a group R^{b} and preferably have the following meanings:

R^{b1} is H;

R^{b4} is H, halogen, CH₃ or OCH₃, in particular H, F, Cl, Br, CH₃ or OCH₃.

In a further embodiment of the compounds of the formula I, X and Y are N and V and W are C—R^{b}. These compounds correspond to the formula I.11

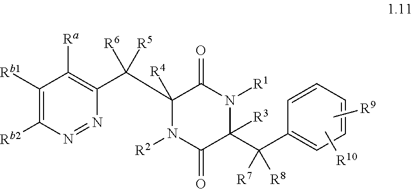

in which the groups R^{b1} and R^{b2} each correspond to a group R^{b} and preferably have the following meanings:

R^{b1} is H_{;}

R^{b2} is H, halogen, halomethyl, in particular H, C₁, CF₃.

Particularly preferred aspects of the compounds of the formula I relate to those of each of the formulae I.1 to I.11 in which the variables R^{a} and R¹ to R¹⁰ have the meanings preferred for formula I.

In a first preferred embodiment of the invention, R^{a} is CN or NO₂.

R^{a} is in particular CN, NO₂ or a 5- or 6-membered heteroaromatic group, as defined above, which preferably has either 1, 2 or 3 nitrogen atoms or 1 oxygen or 1 sulfur atom and optionally 1 or 2 nitrogen atoms as ring members and which is unsubstituted or may have 1 or 2 substituents selected from the group consisting of R^{aa} and/or R^{a1}.

In a further preferred embodiment of the invention, R^{a} is a 5- or 6-membered heterocycle as defined above, which preferably has either 1, 2, 3 or 4 nitrogen atoms or 1 oxygen or 1 sulfur atom and optionally 1 or 2 nitrogen atoms as ring members and which is unsubstituted or may have 1 or 2 substituents selected from R^{aa}. Preference is given to saturated or partially unsaturated groups attached via nitrogen, such as, for example:

Heteroaromatic groups: pyridazin-3-yl, pyridazin-4-yl, pyrimidin-2-yl, pyrimidin-4-yl, pyrimidin-5-yl, pyrazin-2-yl, 2-furyl, 3-furyl, 2-thienyl, 3-thienyl, pyrazol-1-yl, pyrazol-3-yl, pyrazol-4-yl, isoxazol-3-yl, isoxazol-4-yl, isoxazol-5-yl, isothiazol-3-yl, isothiazol-4-yl, isothiazol-5-yl, imidazol-1-yl, imidazol-2-yl, imidazol-4-yl, oxazol-2-yl, oxazol-4-yl, oxazol-5-yl, thiazol-2-yl, thiazol-4-yl and thiazol-5-yl;

In another aspect, R^{a} is a heteroaromatic group attached via carbon, such as pyrazol-3-yl, imidazol-5-yl, oxazol-2-yl, thiazol-2-yl, thiazol-4-yl, thiazol-5-yl, pyridin-2-yl, pyridin-3-yl, pyridin-4-yl, pyrimidin-2-yl, pyrimidin-4-yl, pyrimidin-5-yl, pyridazin-4-yl, pyrazin-2-yl, [1H]-tetrazol-5-yl and [2H]-tetrazol-5-yl, where each of the heterocycles mentioned here in an exemplary manner may have 1 or 2 substituents selected from R^{aa}. Preferred groups R^{aa} are in particular F, Cl, CN, NO₂, CH₃, ethyl, OCH₃, OC₂H₅, OCHF2, OCF₃ and CF₃.

Preference is also given to compounds of the formula I and their salts in which R^{a} is halogen, in particular Cl or Br.

In a further preferred aspect, R^{a} is NR^{A}R^{B}, where R^{A} and R^{B} independently of one another are hydrogen, alkyl, haloalkyl, alkenyl, alkynyl or alkoxyalkyl or cyanoalkyl.

In a further preferred aspect, R^{a} is C(R^{aa})C(O)R^{a1}, where R^{aa} is in particular CN or a group C(O)R^{a1} and Rat is preferably C₁-C₆-alkoxy.

If R^{a} is cycloalkyl, cyclohexyl and in particular cyclopropyl are preferred groups.

In a further preferred aspect, R^{a} is C₁-C₄-alkyl which may be substituted by C₁-C₆-alkoxy, C₃-C₈-alkenyloxy or C₃-C₈-alkynyloxy.

In a further preferred aspect, R^{a} is C₁-C₄-alkyl, C₂-C₆-alkenyl or C₂-C₆-alkynyl which may be substituted by halogen, CN, NO₂ or NR^{A}R^{B}.

In a further preferred aspect R^{a} is C₁-C₆-alkoxy which may be substituted by halogen, such as OCH₃, OC₂H₅, OCHF₂ or OCF₃.

The group R^{b} is preferably H, F, Cl, C₁-C₂-alkyl, C₁-C₂-fluoroalkyl, CH═CH₂, C₁-C₂-alkoxy or C₁-C₂-fluoroalkoxy, in particular F, Cl, CH₃, C₂H₅, OCH₃, CH═CH₂ or OCF₃.

In a particularly preferred embodiment, R^{b} is halogen, in particular Cl or F, methyl or methoxy which is located in the ortho-position to the point of attachment of the heteroaryl ring (R^{b4}).

From among the compounds of the formula I in which R^{b} is halogen, preference is also given to those compounds in which R^{b} is located in the para-position to the group R^{a}.

From among the compounds of the formula I in which R^{b} is halogen, preference is given to those compounds in which R^{b} is F or Cl.

In one aspect of the compounds of the formula I, R¹ is alkyl, in particular methyl, which is substituted by a group selected from CN, NO₂, halogen, C₁-C₄-alkoxy, C(═O)—R^{a1}. C₃-C₆-cycloalkyl and optionally substituted phenyl.

In a further aspect of the compounds of the formula I, R¹ is NH₂ or SO₂R^{y}.

In a further aspect of the compounds of the formula I, R¹ is CH₂CH═CHCH₃, CH₂CH₂CH═CH₂, CH₂C≡CCH₃, CH₂CH₂C≡CH.

In a further aspect of the compounds of the formula I, R¹ is substituted C₃-C₄-alkenyl or C₃-C₄-alkynyl, in particular substituted by halogen. If R¹ is a group substituted by R^{aa}, the meaning R¹ of the respective group R^{aa} is of course different.

R² is preferably CH₃.

R³ is preferably C₁-C₃-alkyl, C₁-C₂-fluoroalkyl or C₂-C₃-alkenyl, in particular CH₃, C₂H₅, n-propyl, CF₃ or allyl and preferably CH₃ or C₂H₅.

Preference is also given to compounds of the formula I in which R⁶ is a group C(═O)R¹¹ in which R¹¹ has one of the meanings mentioned above and is in particular H, C₁-C₄-alkyl, preferably CH₃ or C₂H₅, or is C₁-C₄-haloalkyl, preferably C₁-C₂-fluoroalkyl, such as CF₃.

Preferably, at least one and in particular both groups R⁷ and R⁸ is/are H.

From among the compounds of the formula I in which R⁹ is a group different from H, preference is given to those compounds in which R⁹ is located in the para-position to the group CR⁷R⁸.

From among the compounds of the formula I in which R⁹ is a group different from H, preference is given to those compounds in which R⁹ is located in the meta-position to the point of attachment and is preferably halogen, in particular F or Cl. In another, likewise preferred embodiment, R⁹ is H.

R¹⁰ is preferably H or halogen, such as Cl or F, in particular F. In a preferred aspect, R¹⁰ is located in the ortho- or para-position. Particularly preferably, R¹⁹ is H.

In the group C(O)R¹¹, R¹¹ is preferably H, C₁-C₄-alkyl or C₁-C₄-haloalkyl.

From among the compounds of the formula I and their salts, preference is given to the compounds of the formula I.A and their agriculturally suitable salts:

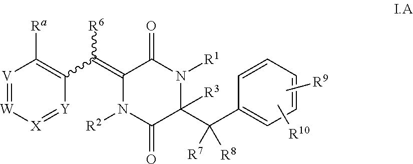

in which the variables have one of the meanings given for formula I, in particular the meanings given as being preferred.

In formula I and in particular in formula I.A and the subformulae derived therefrom, the groups R¹, R², R³, R⁶, R⁷, R⁸, R⁹, R¹⁰, R^{a} and R^{b} independently of one another, but preferably in combination, have the meanings below:
R¹ is NH₂, SO₂CH₃, CH₂CO₂—C₁-C₄-alkyl, CH₂CH₂C≡CCH₃, CH₂C(CH₃)═CH₂, CH₂CH═CHCH₃, C₃-C₄-haloalkenyl, CH₂-c-C₃H₅, optionally substituted benzyl, C₁-C₂-alkyl substituted by CN, Cl, F;

R² is CH₃;

R³ is C₁-C₄-alkyl, OH, CH₂OH, NH₂, C(O)R¹¹, where R¹¹ is C₁-C₄-alkoxy, in particular CH₃ or C₂H₅;

R⁶ is H, CH₃ or C₂H₅, in particular H;

R⁷, R⁸ are H;

R⁹ is H, halogen, OH, C₁-C₄-alkyl, C₁-C₄-alkylcarbonyloxy, in particular H or 3-halogen, OH, CH₃, OCOCH₃, especially H or 3-F;

R¹⁰ is H or F;

R^{a} is halogen, CN, NO₂, C₁-C₄-alkyl, C₁-C₄-alkoxy, C₁-C₄-thioalkyl, NR^{A}R^{B}, haloalkyl, haloalkoxy, in particular Cl, CN, NO₂, CH₃, OCH₃, OC₂H₅, SCH₃, or NR^{A}R^{B}, where R^{A} and R^{B} together with the nitrogen atom form a six-membered saturated heterocycle, such as, for example, N-morpholinyl; and

R^{b} is H, F, Cl, Br, CH₃, OCH₃, halomethyl, in particular, depending on the position of the group R^{b}, the meanings mentioned further above for R^{b1}, R^{b2}, R^{b3}, R^{b4}.

In particularly preferred aspects, the compounds I.A have the preferred features of the formulae I.1 to I.11. Accordingly, they are referred to as formulae I.1A to I.11A.

A further embodiment of the compounds of the formula I relates to those where R⁴ and R⁵ are H. Such compounds correspond to the formula I.B

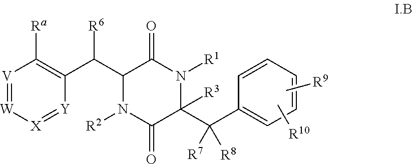

In particularly preferred aspects, the compounds I.B have the preferred features of the formulae I.1 to I.11. Accordingly, they are referred to as formulae I.1B to I.11B.

At the carbon atom which carries the group R³, the compounds of the formula I have a center of chirality. A preferred embodiment of the invention relates to the pure enantiomers of the formula I-S shown below

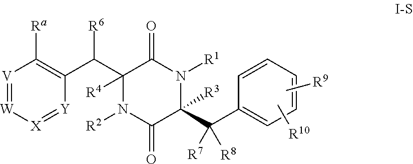

in which the variables have one of the meanings given above, in particular one of the meanings given as being preferred or as being particularly preferred, and also to enantiomer mixtures having an enantiomeric excess of the enantiomer of the formula I-S.

In particularly preferred aspects, the compounds I-S have the preferred features of the formulae I.1 to I.11. Accordingly, they are referred to as formulae I.1-S to I.11-S.

If R⁴ does not represent a bond to R⁵, the compounds I also have a center of chirality at the carbon atom which carries the group R⁴. For the compounds of the formula I, in particular those of the formula I-S, the S configuration at this position is preferred.

Enantiomeric excess preferably means an ee value of at least 70%, in particular at least 80% and preferably at least 90%. Preference is also given to the agriculturally suitable salts of the enantiomers I-S and enantiomer mixtures of the salts having an enantiomeric excess of the enantiomer of the formula I-S.

Another embodiment, which is likewise preferred, relates to the racemates of I and their salts.

A particularly preferred embodiment relates to the pure enantiomers of the formula I.A-S given below in which the variables have one of the meanings given above, in particular one of the meanings given as being preferred or as being particularly preferred, and also to enantiomer mixtures having an enantiomeric excess of the enantiomer of the formula I.A-S.

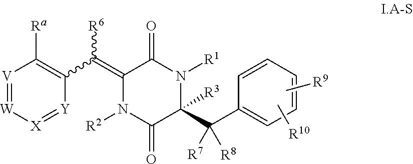

Preference is also given to the agriculturally suitable salts of the enantiomers I.A-S and to enantiomer mixtures of the salts having an enantiomeric excess of the enantiomer of the formula I.A-S.

Another particularly preferred embodiment of the invention relates to the racemates of I.A and their salts.

From among the compounds of the formulae I.A and the subformulae derived therefrom, preference is given to those compounds in which the exo double bond at the piperazine ring has the (Z) configuration. Preference is also given to mixtures of the (E) isomer with the (Z) isomer in which the Z isomer is present in excess, in particular to isomer mixtures having an E/Z ratio of not more than 1:2, in particular not more than 1:5.

In particular with a view to their use, preference is given to the compounds of the formula I compiled in the tables below, which compounds correspond to the formulae I.1A′, I.1B′, I.2A′ and I.2B′, respectively. The groups mentioned for a substituent in the tables are furthermore per se, independently of the combination in which they are mentioned, a particularly preferred aspect of the substituent in question.

Table 1

Compounds of the formula I in which R^{a} is CN, R^{b2}, R^{b3} and R^{b4} are H and the combination of R¹, R³, R⁹ and R¹⁰ for a compound corresponds in each case to one row of table A

Table 2

Compounds of the formula I in which R^{a} is CN, R^{b2} and R^{b3} are H and R^{b4} is F and the combination of R¹, R³, R⁹ and R¹⁰ for a compound corresponds in each case to one row of table A

Table 3

Compounds of the formula I in which R^{a} is CN, R^{b2} and R^{b3} are H and R^{b4} is Cl and the combination of R¹, R³, R⁹ and R¹⁰ for a compound corresponds in each case to one row of table A

Table 4

Compounds of the formula I in which R^{a} is CN, R^{b2} and R^{b3} are H and R^{b4} is Br and the combination of R¹, R³, R⁹ and R¹⁰ for a compound corresponds in each case to one row of table A

Table 5

Compounds of the formula I in which R^{a} is CN, R^{b2} and R^{b3} are H and R^{b4} is CH₃ and the combination of R¹, R³, R⁹ and R¹⁰ for a compound corresponds in each case to one row of table A

Table 6

Compounds of the formula I in which R^{a} is CN, R^{b2} and R^{b3} are H and R^{b4} is OCH₃ and the combination of R¹, R³, R⁹ and R¹⁰ for a compound corresponds in each case to one row of table A

Table 7

Compounds of the formula I in which R^{a} is CN, R^{b2} is H, R^{b3} is F and R^{b4} is H and the combination of R¹, R³, R⁹ and R¹⁰ for a compound corresponds in each case to one row of table A

Table 8

Compounds of the formula I in which R^{a} is CN, R^{b2} is H, R^{b3} and R^{b4} are F and the combination of R¹, R³, R⁹ and R¹⁰ for a compound corresponds in each case to one row of table A

Table 9

Compounds of the formula I in which R^{a} is CN, R^{b2} is H, R^{b3} is F and R^{b4} is Cl and the combination of R¹, R³, R⁹ and R¹⁰ for a compound corresponds in each case to one row of table A

Table 10

Compounds of the formula I in which R^{a} is CN, R^{b2} is H, R^{b3} is F and R^{b4} is Br and the combination of R¹, R³, R⁹ and R¹⁰ for a compound corresponds in each case to one row of table A

Table 11

Compounds of the formula I in which R^{a} is CN, R^{b2} is H, R^{b3} is F and R^{b4} is CH₃ and the 1.5 combination of R¹, R³, R⁹ and R¹⁰ for a compound corresponds in each case to one row of table A

Table 12

Compounds of the formula I in which R^{a} is CN, R^{b2} is H, R^{b3} is F and R^{b4} is OCH₃ and the combination of R¹, R³, R⁹ and R¹⁰ for a compound corresponds in each case to one row of table A

Table 13

Compounds of the formula I in which R^{a} is Cl, R^{b2}, R^{b3} and R^{b4} are H and the combination of R¹, R³, R⁹ and R¹⁰ for a compound corresponds in each case to one row of table A

Table 14

Compounds of the formula I in which R^{a} is Cl, R^{b2} and R^{b3} are H and R^{b4} is F and the combination of R¹, R³, R⁹ and R¹⁰ for a compound corresponds in each case to one row of table A

Table 15

Compounds of the formula I in which R^{a} is Cl, R^{b2} and R^{b3} are H and R^{b4} is Cl and the combination of R¹, R³, R⁹ and R¹⁰ for a compound corresponds in each case to one row of table A

Table 16

Compounds of the formula I in which R^{a} is Cl, R^{b2} and R^{b3} are H and R^{b4} is Br and the combination of R¹, R³, R⁹ and R¹⁰ for a compound corresponds in each case to one row of table A

Table 17

Compounds of the formula I in which R^{a} is Cl, R^{b2} and R^{b3} are H and R^{b4} is CH₃ and the combination of R¹, R³, R⁹ and R¹⁰ for a compound corresponds in each case to one row of table A

Table 18

Compounds of the formula I in which R^{a} is Cl, R^{b2} and R^{b3} are H and R^{b4} is OCH₃ and the combination of R¹, R³, R⁹ and R¹⁰ for a compound corresponds in each case to one row of table A

Table 19

Compounds of the formula I in which R^{a} is Cl, R^{b2} is H, R^{b3} is F and R^{b4} is H and the combination of R¹, R³, R⁹ and R¹⁰ for a compound corresponds in each case to one row of table A

Table 20

Compounds of the formula I in which R^{a} is Cl, R^{b2} is H, R^{b3} and R^{b4} are F and the combination of R¹, R³, R⁹ and R¹⁰ for a compound corresponds in each case to one row of table A

Table 21

Compounds of the formula I in which R^{a} is C¹, R^{b2} is H, R^{b3} is F and R^{b4} is Cl and the combination of R¹, R³, R⁹ and R¹⁰ for a compound corresponds in each case to one row of table A

Table 22

Compounds of the formula I in which R^{a} is Cl, R^{b2} is H, R^{b3} is F and R^{b4} is Br and the combination of R¹, R³, R⁹ and R¹⁰ for a compound corresponds in each case to one row of table A

Table 23

Compounds of the formula I in which R^{a} is Cl, R^{b2} is H, R^{b3} is F and R^{b4} is CH₃ and the combination of R¹, R³, R⁹ and R¹⁰ for a compound corresponds in each case to one row of table A

Table 24

Compounds of the formula I in which R^{a} is Cl, R^{b2} is H, R^{b3} is F and R^{b4} is OCH₃ and the combination of R¹, R³, R⁹ and R¹⁰ for a compound corresponds in each case to one row of table A

Table 25

Compounds of the formula I in which R^{a} is NO₂, R^{b2}, R^{b3} and R^{b4} are H and the combination of R¹, R³, R⁹ and R¹⁰ for a compound corresponds in each case to one row of table A

Table 26

Compounds of the formula I in which R^{a} is NO₂, R^{b2} and R^{b3} are H and R^{b4} is F and the combination of R¹, R³, R⁹ and R¹⁰ for a compound corresponds in each case to one row of table A

Table 27

Compounds of the formula I in which R^{a} is NO₂, R^{b2} and R^{b3} are H and R^{b4} is Cl and the combination of R¹, R³, R⁹ and R¹⁰ for a compound corresponds in each case to one row of table A

Table 28

Compounds of the formula I in which R^{a} is NO₂, R^{b2} and R^{b3} are H and R^{b4} is Br and the combination of R¹, R³, R⁹ and R¹⁰ for a compound corresponds in each case to one row of table A

Table 29

Compounds of the formula I in which R^{a} is NO₂, R^{b2} and R^{b3} are H and R^{b4} is CH₃ and the combination of R¹, R³, R⁹ and R¹⁰ for a compound corresponds in each case to one row of table A

Table 30

Compounds of the formula I in which R^{a} is NO₂, R^{b2} and R^{b3} are H and R^{b4} is OCH₃ and the combination of R¹, R³, R⁹ and R¹⁰ for a compound corresponds in each case to one row of table A

Table 31

Compounds of the formula I in which R^{a} is NO₂, R^{b2} is H, R^{b3} is F and R^{b4} is H and the combination of R¹, R³, R⁹ and R¹⁰ for a compound corresponds in each case to one row of table A

Table 32

Compounds of the formula I in which R^{a} is NO₂, R^{b2} is H, R^{b3} and R^{b4} are F and the combination of R¹, R³, R⁹ and R¹⁰ for a compound corresponds in each case to one row of table A

Table 33

Compounds of the formula I in which R^{a} is NO₂, R^{b2} is H, R^{b3} is F and R^{b4} is Cl and the combination of R¹, R³, R⁹ and R¹⁰ for a compound corresponds in each case to one row of table A

Table 34

Compounds of the formula I in which R^{a} is NO₂, R^{b2} is H, R^{b3} is F and R^{b4} is Br and the combination of R¹, R³, R⁹ and R¹⁰ for a compound corresponds in each case to one row of table A

Table 35

Compounds of the formula I in which R^{a} is NO₂, R^{b2} is H, R^{b3} is F and R^{b4} is CH₃ and the combination of R¹, R³, R⁹ and R¹⁰ for a compound corresponds in each case to one row of table A

Table 36

Compounds of the formula I in which R^{a} is NO₂, R^{b2} is H, R^{b3} is F and R^{b4} is OCH₃ and the combination of R¹, R³, R⁹ and R¹⁰ for a compound corresponds in each case to one row of table A

Table 37

Compounds of the formula I in which R^{a} is CH₃, R^{b2} and R^{b3} are H and R^{b4} is H and the combination of R¹, R³, R⁹ and R¹⁰ for a compound corresponds in each case to one row of table A

Table 38

Compounds of the formula I in which R^{a} is CH₃, R^{b2} and R^{b3} are H and R^{b4} is F and the combination of R¹, R³, R⁹ and R¹⁰ for a compound corresponds in each case to one row of table A

Table 39

Compounds of the formula I in which R^{a} is CH₃, R^{b2} and R^{b3} are H and R^{b4} is Cl and the combination of R¹, R³, R⁹ and R¹⁰ for a compound corresponds in each case to one row of table A

Table 40

Compounds of the formula I in which R^{a} is CH₃, R^{b2} and R^{b3} are H and R^{b4} is Br and the combination of R¹, R³, R⁹ and R¹⁰ for a compound corresponds in each case to one row of table A

Table 41

Compounds of the formula I in which R^{a} is CH₃, R^{b2} and R^{b3} are H and R^{b4} is CH₃ and the combination of R¹, R³, R⁹ and R¹⁰ for a compound corresponds in each case to one row of table A

Table 42

Compounds of the formula I in which R^{a} is CH₃, R^{b2} and R^{b3} are H and R^{b4} is OCH₃ and the combination of R¹, R³, R⁹ and R¹⁰ for a compound corresponds in each case to one row of table A

Table 43

Compounds of the formula I in which R^{a} is CH₃, R^{b2} is H, R^{b3} is F and R^{b4} is H and the combination of R¹, R³, R⁹ and R¹⁰ for a compound corresponds in each case to one row of table A

Table 44

Compounds of the formula I in which R^{a} is CH₃, R^{b2} is H, R^{b3} is F and R^{b4} is F and the combination of R¹, R³, R⁹ and R¹⁰ for a compound corresponds in each case to one row of table A

Table 45

Compounds of the formula I in which R^{a} is CH₃, R^{b2} is H, R^{b3} is F and R^{b4} is Cl and the combination of R¹, R³, R⁹ and R¹⁰ for a compound corresponds in each case to one row of table A

Table 46

Compounds of the formula I in which R^{a} is CH₃, R^{b2} is H, R^{b3} is F and R^{b4} is Br and the combination of R¹, R³, R⁹ and R¹⁰ for a compound corresponds in each case to one row of table A

Table 47

Compounds of the formula I in which R^{a} is CH₃, R^{b2} is H, R^{b3} is F and R^{b4} is CH₃ and the combination of R¹, R³, R⁹ and R¹⁰ for a compound corresponds in each case to one row of table A

Table 48

Compounds of the formula I in which R^{a} is CH₃, R^{b2} is H, R^{b3} is F and R^{b4} is OCH₃ and the combination of R¹, R³, R⁹ and R¹⁰ for a compound corresponds in each case to one row of table A

Table 49

Compounds of the formula I in which R^{a} is CF₃, R^{b2} and R^{b3} are H and R^{b4} is H and the combination of R¹, R³, R⁹ and R¹⁰ for a compound corresponds in each case to one row of table A

Table 50

Compounds of the formula I in which R^{a} is CF₃, R^{b2} and R^{b3} are H and R^{b4} is F and the combination of R¹, R³, R⁹ and R¹⁰ for a compound corresponds in each case to one row of table A

Table 51

Compounds of the formula I in which R^{a} is CF₃, R^{b2} and R^{b3} are H and R^{b4} is Cl and the combination of R¹, R³, R⁹ and R¹⁰ for a compound corresponds in each case to one row of table A

Table 52

Compounds of the formula I in which R^{a} is CF₃, R^{b2} and R^{b3} are H and R^{b4} is Br and the combination of R¹, R³, R⁹ and R¹⁰ for a compound corresponds in each case to one row of table A

Table 53

Compounds of the formula I in which R^{a} is CF₃, R^{b2} and R^{b3} are H and R^{b4} is CH₃ and the combination of R¹, R³, R⁹ and R¹⁰ for a compound corresponds in each case to one row of table A

Table 54

Compounds of the formula I in which R^{a} is CF₃, R^{b2} and R^{b3} are H and R^{b4} is OCH₃ and the combination of R¹, R³, R⁹ and R¹⁰ for a compound corresponds in each case to one row of table A

Table 55

Compounds of the formula I in which R^{a} is CF₃, R^{b2} is H, R^{b3} is F and R^{b4} is H and the combination of R¹, R³, R⁹ and R¹⁰ for a compound corresponds in each case to one row of table A

Table 56

Compounds of the formula I in which R^{a} is CF₃, R^{b2} is H, R^{b3} is F and R^{b4} is F and the combination of R¹, R³, R⁹ and R¹⁰ for a compound corresponds in each case to one row of table A

Table 57

Compounds of the formula I in which R^{a} is CF₃, R^{b2} is H, R^{b3} is F and R^{b4} is Cl and the combination of R¹, R³, R⁹ and R¹⁰ for a compound corresponds in each case to one row of table A

Table 58

Compounds of the formula I in which R^{a} is CF₃, R^{b2} is H, R^{b3} is F and R^{b4} is Br and the combination of R¹, R³, R⁹ and R¹⁰ for a compound corresponds in each case to one row of table A

Table 59

Compounds of the formula I in which R^{a} is CF₃, R^{b2} is H, R^{b3} is F and R^{b4} is CH₃ and the combination of R¹, R³, R⁹ and R¹⁰ for a compound corresponds in each case to one row of table A

Table 60

Compounds of the formula I in which R^{a} is CF₃, R^{b2} is H, R^{b3} is F and R^{b4} is OCH₃ and the combination of R¹, R³, R⁹ and R¹⁰ for a compound corresponds in each case to one row of table A

Table 61

Compounds of the formula I in which R^{a} is OCH₃, R^{b2} and R^{b3} are H and R^{b4} is H and the combination of R¹, R³, R⁹ and R¹⁰ for a compound corresponds in each case to one row of table A

Table 62

Compounds of the formula I in which R^{a} is OCH₃, R^{b2} and R^{b3} are H and R^{b4} is F and the combination of R¹, R³, R⁹ and R¹⁰ for a compound corresponds in each case to one row of table A

Table 63

Compounds of the formula I in which R^{a} is OCH₃, R^{b2} and R^{b3} are H and R^{b4} is Cl and the combination of R¹, R³, R⁹ and R¹⁰ for a compound corresponds in each case to one row of table A

Table 64

Compounds of the formula I in which R^{a} is OCH₃, R^{b2} and R^{b3} are H and R^{b4} is Br and the combination of R¹, R³, R⁹ and R¹⁰ for a compound corresponds in each case to one row of table A

Table 65

Compounds of the formula I in which R^{a} is OCH₃, R^{b2} and R^{b3} are H and R^{b4} is CH₃ and the combination of R¹, R³, R⁹ and R¹⁰ for a compound corresponds in each case to one row of table A

Table 66

Compounds of the formula I in which R^{a} is OCH₃, R^{b2} and R^{b3} are H and R^{b4} is OCH₃ and the combination of R¹, R³, R⁹ and R¹⁰ for a compound corresponds in each case to one row of table A

Table 67

Compounds of the formula I in which R^{a} is OCH₃, R^{b2} is H, R^{b3} is F and R^{b4} is H and the combination of R¹, R³, R⁹ and R¹⁰ for a compound corresponds in each case to one row of table A

Table 68

Compounds of the formula I in which R^{a} is OCH₃, R^{b2} is H, R^{b3} is F and R^{b4} is F and the combination of R¹, R³, R⁹ and R¹⁰ for a compound corresponds in each case to one row of table A

Table 69

Compounds of the formula I in which R^{a} is OCH₃, R^{b2} is H, R^{b3} is F and R^{b4} is Cl and the combination of R¹, R³, R⁹ and R¹⁰ for a compound corresponds in each case to one row of table A

Table 70

Compounds of the formula I in which R^{a} is OCH₃, R^{b2} is H, R^{b3} is F and R^{b4} is Br and the combination of R¹, R³, R⁹ and R¹⁰ for a compound corresponds in each case to one row of table A

Table 71

Compounds of the formula I in which R^{a} is OCH₃, R^{b2} is H, R^{b3} is F and R^{b4} is CH₃ and the combination of R¹, R³, R⁹ and R¹⁰ for a compound corresponds in each case to one row of table A

Table 72

Compounds of the formula I in which R^{a} is OCH₃, R^{b2} is H, R^{b3} is F and R^{b4} is OCH₃ and the combination of R¹, R³, R⁹ and R¹⁰ for a compound corresponds in each case to one row of table A

Table 73

Compounds of the formula I in which R^{a} is OC₂H₅, R^{b2} and R^{b3} are H and R^{b4} is H and the combination of R¹, R³, R⁹ and R¹⁰ for a compound corresponds in each case to one row of table A

Table 74

Compounds of the formula I in which R^{a} is OC₂H₅, R^{b2} and R^{b3} are H and R^{b4} is F and the combination of R¹, R³, R⁹ and R¹⁰ for a compound corresponds in each case to one row of table A

Table 75

Compounds of the formula I in which R^{a} is OC₂H₅, R^{b2} and R^{b3} are H and R^{b4} is Cl and the combination of R¹, R³, R⁹ and R¹⁰ for a compound corresponds in each case to one row of table A

Table 76

Compounds of the formula I in which R^{a} is OC₂H₅, R^{b2} and R^{b3} are H and R^{b4} is Br and the combination of R¹, R³, R⁹ and R¹⁰ for a compound corresponds in each case to one row of table A

Table 77

Compounds of the formula I in which R^{a} is OC₂H₅, R^{b2} and R^{b3} are H and R^{b4} is CH₃ and the combination of R¹, R³, R⁹ and R¹⁰ for a compound corresponds in each case to one row of table A

Table 78

Compounds of the formula I in which R^{a} is OC₂H₅, R^{b2} and R^{b3} are H and R^{b4} is OCH₃ and the combination of R¹, R³, R⁹ and R¹⁰ for a compound corresponds in each case to one row of table A

Table 79

Compounds of the formula I in which R^{a} is OC₂H₅, R^{b2} is H, R^{b3} is F and R^{b4} is H and the combination of R¹, R³, R⁹ and R¹⁰ for a compound corresponds in each case to one row of table A

Table 80

Compounds of the formula I in which R^{a} is OC₂H₅, R^{b2} is H, R^{b3} is F and R^{b4} is F and the combination of R¹, R³, R⁹ and R¹⁰ for a compound corresponds in each case to one row of table A

Table 81

Compounds of the formula I in which R^{a} is OC₂H₅, R^{b2} is H, R^{b3} is F and R^{b4} is Cl and the combination of R¹, R³, R⁹ and R¹⁰ for a compound corresponds in each case to one row of table A

Table 82

Compounds of the formula I in which R^{a} is OC₂H₅, R^{b2} is H, R^{b3} is F and R^{b4} is Br and the combination of R¹, R³, R⁹ and R¹⁰ for a compound corresponds in each case to one row of table A

Table 83

Compounds of the formula I in which R^{a} is OC₂H₅, R^{b2} is H, R^{b3} is F and R^{b4} is CH₃ and the combination of R¹, R³, R⁹ and R¹⁰ for a compound corresponds in each case to one row of table A

Table 84

Compounds of the formula I in which R^{a} is OC₂H₅, R^{b2} is H, R^{b3} is F and R^{b4} is OCH₃ and the combination of R¹, R³, R⁹ and R¹⁰ for a compound corresponds in each case to one row of table A

Table 85

Compounds of the formula I in which R^{a} is morpholin-1-yl, R^{b2} and R^{b3} are H and R^{b4} is H and the combination of R¹, R³, R⁹ and R¹⁰ for a compound corresponds in each case to one row of table A

Table 86

Compounds of the formula I in which R^{a} is morpholin-1-yl, R^{b2} and R^{b3} are H and R^{b4} is F and the combination of R¹, R³, R⁹ and R¹⁰ for a compound corresponds in each case to one row of table A

Table 87

Compounds of the formula I in which R^{a} is morpholin-1-yl, R^{b2} and R^{b3} are H and R^{b4} is Cl and the combination of R¹, R³, R⁹ and R¹⁰ for a compound corresponds in each case to one row of table A

Table 88

Compounds of the formula I in which R^{a} is morpholin-1-yl, R^{b2} and R^{b3} are H and R^{b4} is Br and the combination of R¹, R³, R⁹ and R¹⁰ for a compound corresponds in each case to one row of table A

Table 89

Compounds of the formula I in which R^{a} is morpholin-1-yl, R^{b2} and R^{b3} are H and R^{b4} is CH₃ and the combination of R¹, R³, R⁹ and R¹⁰ for a compound corresponds in each case to one row of table A

Table 90

Compounds of the formula I in which R^{a} is morpholin-1-yl, R^{b2} and R^{b3} are H and R^{b4} is OCH₃ and the combination of R¹, R³, R⁹ and R¹⁰ for a compound corresponds in each case to one row of table A

Table 91

Compounds of the formula I in which R^{a} is morpholin-1-yl, R^{b2} is H, R^{b3} is F and R^{b4} is H and the combination of R¹, R³, R⁹ and R¹⁰ for a compound corresponds in each case to one row of table A

Table 92

Compounds of the formula I in which R^{a} is morpholin-1-yl, R^{b2} is H, R^{b3} is F and R^{b4} is F and the combination of R¹, R³, R⁹ and R¹⁰ for a compound corresponds in each case to one row of table A

Table 93

Compounds of the formula I in which R^{a} is morpholin-1-yl, R^{b2} is H, R^{b3} is F and R^{b4} is Cl and the combination of R¹, R³, R⁹ and R¹⁰ for a compound corresponds in each case to one row of table A

Table 94

Compounds of the formula I in which R^{a} is morpholin-1-yl, R^{b2} is H, R^{b3} is F and R^{b4} is Br and the combination of R¹, R³, R⁹ and R¹⁰ for a compound corresponds in each case to one row of table A

Table 95

Compounds of the formula I in which R^{a} is morpholin-1-yl, R^{b2} is H, R^{b3} is F and R^{b4} is CH₃ and the combination of R¹, R³, R⁹ and R¹⁰ for a compound corresponds in each case to one row of table A

Table 96

Compounds of the formula I in which R^{a} is morpholin-1-yl, R^{b2} is H, R^{b3} is F and R^{b4} is OCH₃ and the combination of R¹, R³, R⁹ and R¹⁰ for a compound corresponds in each case to one row of table A

Table 97

Compounds of the formula I in which R^{a} is CN, R^{b2} is F, R^{b3} and R^{b4} are H and the combination of R¹, R³, R⁹ and R¹⁰ for a compound corresponds in each case to one row of table A

Table 98

Compounds of the formula I in which R^{a} is CN, R^{b2} is F, R^{b3} is H and R^{b4} is F and the combination of R¹, R³, R⁹ and R¹⁰ for a compound corresponds in each case to one row of table A

Table 99

Compounds of the formula I in which R^{a} is CN, R^{b2} is F, R^{b3} is H and R^{b4} is Cl and the combination of R¹, R³, R⁹ and R¹⁰ for a compound corresponds in each case to one row of table A

Table 100

Compounds of the formula I in which R^{a} is CN, R^{b2} is F, R^{b3} is H and R^{b4} is Br and the combination of R¹, R³, R⁹ and R¹⁰ for a compound corresponds in each case to one row of table A

Table 101

Compounds of the formula I in which R^{a} is CN, R^{b2} is F, R^{b3} is H and R^{b4} is CH₃ and the combination of R¹, R³, R⁹ and R¹⁰ for a compound corresponds in each case to one row of table A

Table 102

Compounds of the formula I in which R^{a} is CN, R^{b2} is F, R^{b3} is H and R^{b4} is OCH₃ and the combination of R¹, R³, R⁹ and R¹⁰ for a compound corresponds in each case to one row of table A

Table 103

Compounds of the formula I in which R^{a} is CN, R^{b2} is F, R^{b3} is F and R^{b4} is H and the combination of R¹, R³, R⁹ and R¹⁰ for a compound corresponds in each case to one row of table A

Table 104

Compounds of the formula I in which R^{a} is CN, R^{b2} is F, R^{b3} and R^{b4} are F and the combination of R¹, R³, R⁹ and R¹⁰ for a compound corresponds in each case to one row of table A

Table 105

Compounds of the formula I in which R^{a} is CN, R^{b2} is F, R^{b3} is F and R^{b4} is Cl and the combination of R¹, R³, R⁹ and R¹⁰ for a compound corresponds in each case to one row of table A

Table 106

Compounds of the formula I in which R^{a} is CN, R^{b2} is F, R^{b3} is F and R^{b4} is Br and the combination of R¹, R³, R⁹ and R¹⁰ for a compound corresponds in each case to one row of table A

Table 107

Compounds of the formula I in which R^{a} is CN, R^{b2} is F, R^{b3} is F and R^{b4} is CH₃ and the combination of R¹, R³, R⁹ and R¹⁰ for a compound corresponds in each case to one row of table A

Table 108

Compounds of the formula I in which R^{a} is CN, R^{b2} is F, R^{b3} is F and R^{b4} is OCH₃ and the combination of R¹, R³, R⁹ and R¹⁰ for a compound corresponds in each case to one row of table A

Table 109

Compounds of the formula I in which R^{a} is Cl, R^{b2} is F, R^{b3} and R^{b4} are H and the combination of R¹, R³, R⁹ and R¹⁰ for a compound corresponds in each case to one row of table A

Table 110

Compounds of the formula I in which R^{a} is C¹, R^{b2} is F, R^{b3} is H and R^{b4} is F and the combination of R¹, R³, R⁹ and R¹⁰ for a compound corresponds in each case to one row of table A

Table 111

Compounds of the formula I in which R^{a} is Cl, R^{b2} is F, R^{b3} is H and R^{b4} is Cl and the combination of R¹, R³, R⁹ and R¹⁰ for a compound corresponds in each case to one row of table A

Table 112

Compounds of the formula I in which R^{a} is Cl, R^{b2} is F, R^{b3} is H and R^{b4} is Br and the combination of R¹, R³, R⁹ and R¹⁰ for a compound corresponds in each case to one row of table A

Table 113

Compounds of the formula I in which R^{a} is Cl, R^{b2} is F, R^{b3} is H and R^{b4} is CH₃ and the combination of R¹, R³, R⁹ and R¹⁰ for a compound corresponds in each case to one row of table A

Table 114

Compounds of the formula I in which R^{a} is Cl, R^{b2} is F, R^{b3} is H and R^{b4} is OCH₃ and the combination of R¹, R³, R⁹ and R¹⁰ for a compound corresponds in each case to one row of table A

Table 115

Compounds of the formula I in which R^{a} is Cl, R_{b2} is F, R^{b3} is F and R^{b4} is H and the combination of R¹, R³, R⁹ and R¹⁰ for a compound corresponds in each case to one row of table A

Table 116

Compounds of the formula I in which R^{a} is Cl, R^{b2} is F, R^{b3} and R^{b4} are F and the combination of R¹, R³, R⁹ and R¹⁰ for a compound corresponds in each case to one row of table A

Table 117

Compounds of the formula I in which R^{a} is Cl, R^{b2} is F, R^{b3} is F and R^{b4} is Cl and the combination of R¹, R³, R⁹ and R¹⁰ for a compound corresponds in each case to one row of table A

Table 118

Compounds of the formula I in which R^{a} is Cl, R^{b2} is F, R^{b3} is F and R^{b4} is Br and the combination of R¹, R³, R⁹ and R¹⁰ for a compound corresponds in each case to one row of table A

Table 119

Compounds of the formula I in which R^{a} is Cl, R^{b2} is F, R^{b3} is F and R^{b4} is CH₃ and the combination of R¹, R³, R⁹ and R¹⁰ for a compound corresponds in each case to one row of table A

Table 120

Compounds of the formula I in which R^{a} is Cl, R^{b2} is F, R^{b3} is F and R^{b4} is OCH₃ and the combination of R¹, R³, R⁹ and R¹⁰ for a compound corresponds in each case to one row of table A

Table 121

Compounds of the formula I in which R^{a} is NO₂, R^{b2} is F, R^{b3} and R^{b4} are H and the combination of R¹, R³, R⁹ and R¹⁰ for a compound corresponds in each case to one row of table A

Table 122

Compounds of the formula I in which R^{a} is NO₂, R^{b2} is F, R^{b3} is H and R^{b4} is F and the combination of R¹, R³, R⁹ and R¹⁰ for a compound corresponds in each case to one row of table A

Table 123

Compounds of the formula I in which R^{a} is NO₂, R^{b2} is F, R^{b3} is H and R^{b4} is Cl and the combination of R¹, R³, R⁹ and R¹⁰ for a compound corresponds in each case to one row of table A

Table 124

Compounds of the formula I in which R^{a} is NO₂, R^{b2} is F, R^{b3} is H and R^{b4} is Br and the combination of R¹, R³, R⁹ and R¹⁰ for a compound corresponds in each case to one row of table A

Table 125

Compounds of the formula I in which R^{a} is NO₂, R^{b2} is F, R^{b3} is H and R^{b4} is CH₃ and the combination of R¹, R³, R⁹ and R¹⁰ for a compound corresponds in each case to one row of table A

Table 126

Compounds of the formula I in which R^{a} is NO₂, R^{b2} is F, R^{b3} is H and R^{b4} is OCH₃ and the combination of R¹, R³, R⁹ and R¹⁰ for a compound corresponds in each case to one row of table A

Table 127

Compounds of the formula I in which R^{a} is NO₂, R^{b2} is F, R^{b3} is F and R^{b4} is H and the combination of R¹, R³, R⁹ and R¹⁰ for a compound corresponds in each case to one row of table A

Table 128

Compounds of the formula I in which R^{a} is NO₂, R^{b2} is F, R^{b3} and R^{b4} are F and the combination of R¹, R³, R⁹ and R¹⁰ for a compound corresponds in each case to one row of table A

Table 129

Compounds of the formula I in which R^{a} is NO₂, R^{b2} is F, R^{b3} is F and R^{b4} is Cl and the combination of R¹, R³, R⁹ and R¹⁰ for a compound corresponds in each case to one row of table A

Table 130

Compounds of the formula I in which R^{a} is NO₂, R^{b2} is F, R^{b3} is F and R^{b4} is Br and the combination of R¹, R³, R⁹ and R¹⁰ for a compound corresponds in each case to one row of table A

Table 131

Compounds of the formula I in which R^{a} is NO₂, R^{b2} is F, R^{b3} is F and R^{b4} is CH₃ and the combination of R¹, R³, R⁹ and R¹⁰ for a compound corresponds in each case to one row of table A

Table 132

Compounds of the formula I in which R^{a} is NO₂, R^{b2} is F, R^{b3} is F and R^{b4} is OCH₃ and the combination of R¹, R³, R⁹ and R¹⁰ for a compound corresponds in each case to one row of table A

Table 133

Compounds of the formula I in which R^{a} is CH₃, R^{b2} is F, R^{b3} is H and R^{b4} is H and the combination of R¹, R³, R⁹ and R¹⁰ for a compound corresponds in each case to one row of table A

Table 134

Compounds of the formula I in which R^{a} is CH₃, R^{b2} is F, R^{b3} is H and R^{b4} is F and the combination of R¹, R³, R⁹ and R¹⁰ for a compound corresponds in each case to one row of table A

Table 135

Compounds of the formula I in which R^{a} is CH₃, R^{b2} is F, R^{b3} is H and R^{b4} is Cl and the combination of R¹, R³, R⁹ and R¹⁰ for a compound corresponds in each case to one row of table A

Table 136

Compounds of the formula I in which R^{a} is CH₃, R^{b2} is F, R^{b3} is H and R^{b4} is Br and the combination of R¹, R³, R⁹ and R¹⁰ for a compound corresponds in each case to one row of table A

Table 137

Compounds of the formula I in which R^{a} is CH₃, R^{b2} is F, R^{b3} is H and R^{b4} is CH₃ and the combination of R¹, R³, R⁹ and R¹⁰ for a compound corresponds in each case to one row of table A

Table 138

Compounds of the formula I in which R^{a} is CH₃, R^{b2} is F, R^{b3} is H and R^{b4} is OCH₃ and the combination of R¹, R³, R⁹ and R¹⁰ for a compound corresponds in each case to one row of table A

Table 139

Compounds of the formula I in which R^{a} is CH₃, R^{b2} is F, R^{b3} is F and R^{b4} is H and the combination of R¹, R³, R⁹ and R¹⁰ for a compound corresponds in each case to one row of table A

Table 140

Compounds of the formula I in which R^{a} is CH₃, R^{b2} is F, R^{b3} is F and R^{b4} is F and the combination of R¹, R³, R⁹ and R¹⁰ for a compound corresponds in each case to one row of table A

Table 141

Compounds of the formula I in which R^{a} is CH₃, R^{b2} is F, R^{b3} is F and R^{b4} is Cl and the combination of R¹, R³, R⁹ and R¹⁰ for a compound corresponds in each case to one row of table A

Table 142

Compounds of the formula I in which R^{a} is CH₃, R^{b2} is F, R^{b3} is F and R^{b4} is Br and the combination of R¹, R³, R⁹ and R¹⁰ for a compound corresponds in each case to one row of table A

Table 143

Compounds of the formula I in which R^{a} is CH₃, R^{b2} is F, R^{b3} is F and R^{b4} is CH₃ and the combination of R¹, R³, R⁹ and R¹⁰ for a compound corresponds in each case to one row of table A

Table 144

Compounds of the formula I in which R^{a} is CH₃, R^{b2} is F, R^{b3} is F and R^{b4} is OCH₃ and the combination of R¹, R³, R⁹ and R¹⁰ for a compound corresponds in each case to one row of table A

Table 145

Compounds of the formula I in which R^{a} is CF₃, R^{b2} is F, R^{b3} is H and R^{b4} is H and the combination of R¹, R³, R⁹ and R¹⁰ for a compound corresponds in each case to one row of table A

Table 146

Compounds of the formula I in which R^{a} is CF₃, R^{b2} is F, R^{b3} is H and R^{b4} is F and the combination of R¹, R³, R⁹ and R¹⁰ for a compound corresponds in each case to one row of table A

Table 147

Compounds of the formula I in which R^{a} is CF₃, R^{b2} is F, R^{b3} is H and R^{b4} is Cl and the combination of R¹, R³, R⁹ and R¹⁰ for a compound corresponds in each case to one row of table A

Table 148

Compounds of the formula I in which R^{a} is CF₃, R^{b2} is F, R^{b3} is H and R^{b4} is Br and the combination of R¹, R³, R⁹ and R¹⁰ for a compound corresponds in each case to one row of table A

Table 149

Compounds of the formula I in which R^{a} is CF₃, R^{b2} is F, R^{b3} is H and R^{b4} is CH₃ and the combination of R¹, R³, R⁹ and R¹⁰ for a compound corresponds in each case to one row of table A

Table 150

Compounds of the formula I in which R^{a} is CF₃, R^{b2} is F, R^{b3} is H and R^{b4} is OCH₃ and the combination of R¹, R³, R⁹ and R¹⁰ for a compound corresponds in each case to one row of table A

Table 151

Compounds of the formula I in which R^{a} is CF₃, R^{b2} is F, R^{b3} is F and R^{b4} is H and the combination of R¹, R³, R⁹ and R¹⁰ for a compound corresponds in each case to one row of table A

Table 152

Compounds of the formula I in which R^{a} is CF₃, R^{b2} is F, R^{b3} is F and R^{b4} is F and the combination of R¹, R³, R⁹ and R¹⁰ for a compound corresponds in each case to one row of table A

Table 153

Compounds of the formula I in which R^{a} is CF₃, R^{b2} is F, R^{b3} is F and R^{b4} is Cl and the combination of R¹, R³, R⁹ and R¹⁰ for a compound corresponds in each case to one row of table A

Table 154

Compounds of the formula I in which R^{a} is CF₃, R^{b2} is F, R^{b3} is F and R^{b4} is Br and the combination of R¹, R³, R⁹ and R¹⁰ for a compound corresponds in each case to one row of table A

Table 155

Compounds of the formula I in which R^{a} is CF₃, R^{b2} is F, R^{b3} is F and R^{b4} is CH₃ and the combination of R¹, R³, R⁹ and R¹⁰ for a compound corresponds in each case to one row of table A

Table 156

Compounds of the formula I in which R^{a} is CF₃, R^{b2} is F, R^{b3} is F and R^{b4} is OCH₃ and the combination of R¹, R³, R⁹ and R¹⁰ for a compound corresponds in each case to one row of table A

Table 157

Compounds of the formula I in which R^{a} is OCH₃, R^{b2} is F, R^{b3} is H and R^{b4} is H and the combination of R¹, R³, R⁹ and R¹⁰ for a compound corresponds in each case to one row of table A

Table 158

Compounds of the formula I in which R^{a} is OCH₃, R^{b2} is F, R^{b3} is H and R^{b4} is F and the combination of R¹, R³, R⁹ and R¹⁰ for a compound corresponds in each case to one row of table A

Table 159

Compounds of the formula I in which R^{a} is OCH₃, R^{b2} is F, R^{b3} is H and R^{b4} is Cl and the combination of R¹, R³, R⁹ and R¹⁰ for a compound corresponds in each case to one row of table A

Table 160

Compounds of the formula I in which R^{a} is OCH₃, R^{b2} is F, R^{b3} is H and R^{b4} is Br and the combination of R¹, R³, R⁹ and R¹⁰ for a compound corresponds in each case to one row of table A

Table 161

Compounds of the formula I in which R^{a} is OCH₃, R^{b2} is F, R^{b3} is H and R^{b4} is CH₃ and the combination of R¹, R³, R⁹ and R¹⁰ for a compound corresponds in each case to one row of table A

Table 162

Compounds of the formula I in which R^{a} is OCH₃, R^{b2} is F, R^{b3} is H and R^{b4} is OCH₃ and the combination of R¹, R³, R⁹ and R¹⁰ for a compound corresponds in each case to one row of table A

Table 163

Compounds of the formula I in which R^{a} is OCH₃, R^{b2} is F, R^{b3} is F and R^{b4} is H and the combination of R¹, R³, R⁹ and R¹⁰ for a compound corresponds in each case to one row of table A

Table 164

Compounds of the formula I in which R^{a} is OCH₃, R^{b2} is F, R^{b3} is F and R^{b4} is F and the combination of R¹, R³, R⁹ and R¹⁰ for a compound corresponds in each case to one row of table A

Table 165

Compounds of the formula I in which R^{a} is OCH₃, R^{b2} is F, R^{b3} is F and R^{b4} is Cl and the combination of R¹, R³, R⁹ and R¹⁰ for a compound corresponds in each case to one row of table A

Table 166

Compounds of the formula I in which R^{a} is OCH₃, R^{b2} is F, R^{b3} is F and R^{b4} is Br and the combination of R¹, R³, R⁹ and R¹⁰ for a compound corresponds in each case to one row of table A

Table 167

Compounds of the formula I in which R^{a} is OCH₃, R^{b2} is F, R^{b3} is F and R^{b4} is CH₃ and the combination of R¹, R³, R⁹ and R¹⁰ for a compound corresponds in each case to one row of table A

Table 168

Compounds of the formula I in which R^{a} is OCH₃, R^{b2} is F, R^{b3} is F and R^{b4} is OCH₃ and the combination of R¹, R³, R⁹ and R¹⁰ for a compound corresponds in each case to one row of table A

Table 169

Compounds of the formula I in which R^{a} is OC₂H₅, R^{b2} is F, R^{b3} is H and R^{b4} is H and the combination of R¹, R³, R⁹ and R¹⁰ for a compound corresponds in each case to one row of table A

Table 170

Compounds of the formula I in which R^{a} is OC₂H₅, R^{b2} is F, R^{b3} is H and R^{b4} is F and the combination of R¹, R³, R⁹ and R¹⁰ for a compound corresponds in each case to one row of table A

Table 171

Compounds of the formula I in which R^{a} is OC₂H₅, R^{b2} is F, R^{b3} is H and R^{b4} is Cl and the combination of R¹, R³, R⁹ and R¹⁰ for a compound corresponds in each case to one row of table A

Table 172

Compounds of the formula I in which R^{a} is OC₂H₅, R^{b2} is F, R^{b3} is H and R^{b4} is Br and the combination of R¹, R³, R⁹ and R¹⁰ for a compound corresponds in each case to one row of table A

Table 173

Compounds of the formula I in which R^{a} is OC₂H₅, R^{b2} is F, R^{b3} is H and R^{b4} is CH₃ and the combination of R¹, R³, R⁹ and R¹⁰ for a compound corresponds in each case to one row of table A

Table 174

Compounds of the formula I in which R^{a} is OC₂H₅, R^{b2} is F, R^{b3} is H and R^{b4} is OCH₃ and the combination of R¹, R³, R⁹ and R¹⁰ for a compound corresponds in each case to one row of table A

Table 175

Compounds of the formula I in which R^{a} is OC₂H₅, R^{b2} is F, R^{b3} is F and R^{b4} is H and the combination of R¹, R³, R⁹ and R¹⁰ for a compound corresponds in each case to one row of table A

Table 176

Compounds of the formula I in which R^{a} is OC₂H₅, R^{b2} is F, R^{b3} is F and R^{b4} is F and the combination of R¹, R³, R⁹ and R¹⁰ for a compound corresponds in each case to one row of table A

Table 177

Compounds of the formula I in which R^{a} is OC₂H₅, R^{b2} is F, R^{b3} is F and R^{b4} is Cl and the combination of R¹, R³, R⁹ and R¹⁰ for a compound corresponds in each case to one row of table A

Table 178

Compounds of the formula I in which R^{a} is OC₂H₅, R^{b2} is F, R^{b3} is F and R^{b4} is Br and the combination of R¹, R³, R⁹ and R¹⁰ for a compound corresponds in each case to one row of table A

Table 179

Compounds of the formula I in which R^{a} is OC₂H₅, R^{b2} is F, R^{b3} is F and R^{b4} is CH₃ and the combination of R¹, R³, R⁹ and R¹⁰ for a compound corresponds in each case to one row of table A

Table 180

Compounds of the formula I in which R^{a} is OC₂H₅, R^{b2} is F, R^{b3} is F and R^{b4} is OCH₃ and the combination of R¹, R³, R⁹ and R¹⁰ for a compound corresponds in each case to one row of table A

Table 181

Compounds of the formula I in which R^{a} is morpholin-1-yl, R^{b2} is F and R^{b3} is H and R^{b4} is H and the combination of R¹, R³, R⁹ and R¹⁰ for a compound corresponds in each case to one row of table A

Table 182

Compounds of the formula I in which R^{a} is morpholin-1-yl, R^{b2} is F and R^{b3} is H and R^{b4} is F and the combination of R¹, R³, R⁹ and R¹⁰ for a compound corresponds in each case to one row of table A

Table 183

Compounds of the formula I in which R^{a} is morpholin-1-yl, R^{b2} is F and R^{b3} is H and R^{b4} is Cl and the combination of R¹, R³, R⁹ and R¹⁰ for a compound corresponds in each case to one row of table A

Table 184

Compounds of the formula I in which R^{a} is morpholin-1-yl, R^{b2} is F and R^{b3} is H and R^{b4} is Br and the combination of R¹, R³, R⁹ and R¹⁰ for a compound corresponds in each case to one row of table A

Table 185

Compounds of the formula I in which R^{a} is morpholin-1-yl, R^{b2} is F and R^{b3} is H and R^{b4} is CH₃ and the combination of R¹, R³, R⁹ and R¹⁰ for a compound corresponds in each case to one row of table A

Table 186

Compounds of the formula I in which R^{a} is morpholin-1-yl, R^{b2} is F and R^{b3} is H and R^{b4} is OCH₃ and the combination of R¹, R³, R⁹ and R¹⁰ for a compound corresponds in each case to one row of table A

Table 187

Compounds of the formula I in which R^{a} is morpholin-1-yl, R^{b2} is F, R^{b3} is F and R^{b4} is H and the combination of R¹, R³, R⁹ and R¹⁰ for a compound corresponds in each case to one row of table A

Table 188

Compounds of the formula I in which R^{a} is morpholin-1-yl, R^{b2} is F, R^{b3} is F and R^{b4} is F and the combination of R¹, R³, R⁹ and R¹⁰ for a compound corresponds in each case to one row of table A

Table 189

Compounds of the formula I in which R^{a} is morpholin-1-yl, R^{b2} is F, R^{b3} is F and R^{b4} is Cl and the combination of R¹, R³, R⁹ and R¹⁰ for a compound corresponds in each case to one row of table A

Table 190

Compounds of the formula I in which R^{a} is morpholin-1-yl, R^{b2} is F, R^{b3} is F and R^{b4} is Br and the combination of R¹, R³, R⁹ and R¹⁰ for a compound corresponds in each case to one row of table A

Table 191

Compounds of the formula I in which R^{a} is morpholin-1-yl, R^{b2} is F, R^{b3} is F and R^{b4} is CH₃ and the combination of R¹, R³, R⁹ and R¹⁰ for a compound corresponds in each case to one row of table A

Table 192

Compounds of the formula I in which R^{a} is morpholin-1-yl, R^{b2} is F, R^{b3} is F and R^{b4} is OCH₃ and the combination of R¹, R³, R⁹ and R¹⁰ for a compound corresponds in each case to one row of table A

Table 193

Compounds of the formula I in which R^{a} is CN, R^{b2} is Cl, R^{b3} and R^{b4} are H and the combination of R¹, R³, R⁹ and R¹⁰ for a compound corresponds in each case to one row of table A

Table 194

Compounds of the formula I in which R^{a} is CN, R^{b2} is Cl, R^{b3} is H and R^{b4} is F and the combination of R¹, R³, R⁹ and R¹⁰ for a compound corresponds in each case to one row of table A

Table 195

Compounds of the formula I in which R^{a} is CN, R^{b2} is Cl, R^{b3} is H and R^{b4} is Cl and the combination of R¹, R³, R⁹ and R¹⁰ for a compound corresponds in each case to one row of table A

Table 196

Compounds of the formula I in which R^{a} is CN, R^{b2} is Cl, R^{b3} is H and R^{b4} is Br and the combination of R¹, R³, R⁹ and R¹⁰ for a compound corresponds in each case to one row of table A

Table 197

Compounds of the formula I in which R^{a} is CN, R^{b2} is Cl, R^{b3} is H and R^{b4} is CH₃ and the combination of R¹, R³, R⁹ and R¹⁰ for a compound corresponds in each case to one row of table A

Table 198

Compounds of the formula I in which R^{a} is CN, R^{b2} is Cl, R^{b3} is H and R^{b4} is OCH₃ and the combination of R¹, R³, R⁹ and R¹⁰ for a compound corresponds in each case to one row of table A

Table 199

Compounds of the formula I in which R^{a} is CN, R^{b2} is Cl, R^{b3} is F and R^{b4} is H and the combination of R¹, R³, R⁹ and R¹⁰ for a compound corresponds in each case to one row of table A

Table 200

Compounds of the formula I in which R^{a} is CN, R^{b2} is Cl, R^{b3} and R^{b4} are F and the combination of R¹, R³, R⁹ and R¹⁰ for a compound corresponds in each case to one row of table A

Table 201

Compounds of the formula I in which R^{a} is CN, R^{b2} is Cl, R^{b3} is F and R^{b4} is Cl and the combination of R¹, R³, R⁹ and R¹⁰ for a compound corresponds in each case to one row of table A

Table 202

Compounds of the formula I in which R^{a} is CN, R^{b2} is Cl, R^{b3} is F and R^{b4} is Br and the combination of R¹, R³, R⁹ and R¹⁰ for a compound corresponds in each case to one row of table A

Table 203

Compounds of the formula I in which R^{a} is CN, R^{b2} is Cl, R^{b3} is F and R^{b4} is CH₃ and the combination of R¹, R³, R⁹ and R¹⁰ for a compound corresponds in each case to one row of table A

Table 204

Compounds of the formula I in which R^{a} is CN, R^{b2} is Cl, R^{b3} is F and R^{b4} is OCH₃ and the combination of R¹, R³, R⁹ and R¹⁰ for a compound corresponds in each case to one row of table A

Table 205

Compounds of the formula I in which R^{a} is C¹, R^{b2} is C¹, R^{b3} and R^{b4} are H and the combination of R¹, R³, R⁹ and R¹⁰ for a compound corresponds in each case to one row of table A

Table 206

Compounds of the formula I in which R^{a} is C¹, R^{b2} is C¹, R^{b3} is H and R^{b4} is F and the combination of R¹, R³, R⁹ and R¹⁰ for a compound corresponds in each case to one row of table A

Table 207

Compounds of the formula I in which R^{a} is C¹, R^{b2} is C¹, R^{b3} is H and R^{b4} is Cl and the combination of R¹, R³, R⁹ and R¹⁰ for a compound corresponds in each case to one row of table A

Table 208

Compounds of the formula I in which R^{a} is Cl, R^{b2} is Cl, R^{b3} is H and R^{b4} is Br and the combination of R¹, R³, R⁹ and R¹⁰ for a compound corresponds in each case to one row of table A

Table 209

Compounds of the formula I in which R^{a} is Cl, R^{b2} is Cl, R^{b3} is H and R^{b4} is CH₃ and the combination of R¹, R³, R⁹ and R¹⁰ for a compound corresponds in each case to one row of table A

Table 210

Compounds of the formula I in which R^{a} is Cl, R^{b2} is Cl, R^{b3} is H and R^{b4} is OCH₃ and the combination of R¹, R³, R⁹ and R¹⁰ for a compound corresponds in each case to one row of table A

Table 211

Compounds of the formula I in which R^{a} is Cl, R^{b2} is Cl, R^{b3} is F and R^{b4} is H and the combination of R¹, R³, R⁹ and R¹⁰ for a compound corresponds in each case to one row of table A

Table 212

Compounds of the formula I in which R^{a} is Cl, R^{b2} is Cl, R^{b3} and R^{b4} are F and the combination of R¹, R³, R⁹ and R¹⁰ for a compound corresponds in each case to one row of table A

Table 213

Compounds of the formula I in which R^{a} is Cl, R^{b2} is Cl, R^{b3} is F and R^{b4} is Cl and the combination of R¹, R³, R⁹ and R¹⁰ for a compound corresponds in each case to one row of table A

Table 214

Compounds of the formula I in which R^{a} is Cl, R^{b2} is Cl, R^{b3} is F and R^{b4} is Br and the combination of R¹, R³, R⁹ and R¹⁰ for a compound corresponds in each case to one row of table A

Table 215

Compounds of the formula I in which R^{a} is Cl, R^{b2} is Cl, R^{b3} is F and R^{b4} is CH₃ and the combination of R¹, R³, R⁹ and R¹⁰ for a compound corresponds in each case to one row of table A

Table 216

Compounds of the formula I in which R^{a} is Cl, R^{b2} is Cl, R^{b3} is F and R^{b4} is OCH₃ and the combination of R¹, R³, R⁹ and R¹⁰ for a compound corresponds in each case to one row of table A

Table 217

Compounds of the formula I in which R^{a} is NO₂, R^{b2} is Cl, R^{b3} and R^{b4} are H and the combination of R¹, R³, R⁹ and R¹⁰ for a compound corresponds in each case to one row of table A

Table 218

Compounds of the formula I in which R^{a} is NO₂, R^{b2} is Cl, R^{b3} is H and R^{b4} is F and the combination of R¹, R³, R⁹ and R¹⁰ for a compound corresponds in each case to one row of table A

Table 219

Compounds of the formula I in which R^{a} is NO₂, R^{b2} is Cl, R^{b3} is H and R^{b4} is Cl and the combination of R¹, R³, R⁹ and R¹⁰ for a compound corresponds in each case to one row of table A

Table 220

Compounds of the formula I in which R^{a} is NO₂, R^{b2} is Cl, R^{b3} is H and R^{b4} is Br and the combination of R¹, R³, R⁹ and R¹⁰ for a compound corresponds in each case to one row of table A

Table 221

Compounds of the formula I in which R^{a} is NO₂, R^{b2} is Cl, R^{b3} is H and R^{b4} is CH₃ and the combination of R¹, R³, R⁹ and R¹⁰ for a compound corresponds in each case to one row of table A

Table 222

Compounds of the formula I in which R^{a} is NO₂, R^{b2} is Cl, R^{b3} is H and R^{b4} is OCH₃ and the combination of R¹, R³, R⁹ and R¹⁰ for a compound corresponds in each case to one row of table A

Table 223

Compounds of the formula I in which R^{a} is NO₂, R^{b2} is Cl, R^{b3} is F and R^{b4} is H and the combination of R¹, R³, R⁹ and R¹⁰ for a compound corresponds in each case to one row of table A

Table 224

Compounds of the formula I in which R^{a} is NO₂, R^{b2} is Cl, R^{b3} and R^{b4} are F and the combination of R¹, R³, R⁹ and R¹⁰ for a compound corresponds in each case to one row of table A

Table 225

Compounds of the formula I in which R^{a} is NO₂, R^{b2} is Cl, R^{b3} is F and R^{b4} is Cl and the combination of R¹, R³, R⁹ and R¹⁰ for a compound corresponds in each case to one row of table A

Table 226

Compounds of the formula I in which R^{a} is NO₂, R^{b2} is Cl, R^{b3} is F and R^{b4} is Br and the combination of R¹, R³, R⁹ and R¹⁰ for a compound corresponds in each case to one row of table A

Table 227

Compounds of the formula I in which R^{a} is NO₂, R^{b2} is Cl, R^{b3} is F and R^{b4} is CH₃ and the combination of R¹, R³, R⁹ and R¹⁰ for a compound corresponds in each case to one row of table A

Table 228

Compounds of the formula I in which R^{a} is NO₂, R^{b2} is Cl, R^{b3} is F and R^{b4} is OCH₃ and the combination of R¹, R³, R⁹ and R¹⁰ for a compound corresponds in each case to one row of table A

Table 229

Compounds of the formula I in which R^{a} is CH₃, R^{b2} is Cl, R^{b3} is H and R^{b4} is H and the combination of R¹, R³, R⁹ and R¹⁰ for a compound corresponds in each case to one row of table A

Table 230

Compounds of the formula I in which R^{a} is CH₃, R^{b2} is Cl, R^{b3} is H and R^{b4} is F and the combination of R¹, R³, R⁹ and R¹⁰ for a compound corresponds in each case to one row of table A

Table 231

Compounds of the formula I in which R^{a} is CH₃, R^{b2} is Cl, R^{b3} is H and R^{b4} is Cl and the combination of R¹, R³, R⁹ and R¹⁰ for a compound corresponds in each case to one row of table A

Table 232

Compounds of the formula I in which R^{a} is CH₃, R^{b2} is Cl, R^{b3} is H and R^{b4} is Br and the combination of R¹, R³, R⁹ and R¹⁰ for a compound corresponds in each case to one row of table A

Table 233

Compounds of the formula I in which R^{a} is CH₃, R^{b2} is Cl, R^{b3} is H and R^{b4} is CH₃ and the combination of R¹, R³, R⁹ and R¹⁰ for a compound corresponds in each case to one row of table A

Table 234

Compounds of the formula I in which R^{a} is CH₃, R^{b2} is Cl, R^{b3} is H and R^{b4} is OCH₃ and the combination of R¹, R³, R⁹ and R¹⁰ for a compound corresponds in each case to one row of table A

Table 235

Compounds of the formula I in which R^{a} is CH₃, R^{b2} is Cl, R^{b3} is F and R^{b4} is H and the combination of R¹, R³, R⁹ and R¹⁰ for a compound corresponds in each case to one row of table A

Table 236

Compounds of the formula I in which R^{a} is CH₃, R^{b2} is Cl, R^{b3} is F and R^{b4} is F and the combination of R¹, R³, R⁹ and R¹⁰ for a compound corresponds in each case to one row of table A

Table 237

Compounds of the formula I in which R^{a} is CH₃, R^{b2} is Cl, R^{b3} is F and R^{b4} is Cl and the combination of R¹, R³, R⁹ and R¹⁰ for a compound corresponds in each case to one row of table A

Table 238

Compounds of the formula I in which R^{a} is CH₃, R^{b2} is Cl, R^{b3} is F and R^{b4} is Br and the combination of R¹, R³, R⁹ and R¹⁰ for a compound corresponds in each case to one row of table A

Table 239

Compounds of the formula I in which R^{a} is CH₃, R^{b2} is C¹, R^{b3} is F and R^{b4} is CH₃ and the combination of R¹, R³, R⁹ and R¹⁰ for a compound corresponds in each case to one row of table A

Table 240

Compounds of the formula I in which R^{a} is CH₃, R^{b2} is C¹, R^{b3} is F and R^{b4} is OCH₃ and the combination of R¹, R³, R⁹ and R¹⁰ for a compound corresponds in each case to one row of table A

Table 241

Compounds of the formula I in which R^{a} is CF₃, R^{b2} is C¹, R^{b3} is H and R^{b4} is H and the combination of R¹, R³, R⁹ and R¹⁰ for a compound corresponds in each case to one row of table A

Table 242

Compounds of the formula I in which R^{a} is CF₃, R^{b2} is C¹, R^{b3} is H and R^{b4} is F and the combination of R¹, R³, R⁹ and R¹⁰ for a compound corresponds in each case to one row of table A

Table 243

Compounds of the formula I in which R^{a} is CF₃, R^{b2} is C¹, R^{b3} is H and R^{b4} is Cl and the combination of R¹, R³, R⁹ and R¹⁰ for a compound corresponds in each case to one row of table A

Table 244

Compounds of the formula I in which R^{a} is CF₃, R^{b2} is C¹, R^{b3} is H and R^{b4} is Br and the combination of R¹, R³, R⁹ and R¹⁰ for a compound corresponds in each case to one row of table A

Table 245

Compounds of the formula I in which R^{a} is CF₃, R^{b2} is C¹, R^{b3} is H and R^{b4} is CH₃ and the combination of R¹, R³, R⁹ and R¹⁰ for a compound corresponds in each case to one row of table A

Table 246

Compounds of the formula I in which R^{a} is CF₃, R^{b2} is F, R^{b3} is H and R^{b4} is OCH₃ and the combination of R¹, R³, R⁹ and R¹⁰ for a compound corresponds in each case to one row of table A

Table 247

Compounds of the formula I in which R^{a} is CF₃, R^{b2} is C¹, R^{b3} is F and R^{b4} is H and the combination of R¹, R³, R⁹ and R¹⁰ for a compound corresponds in each case to one row of table A

Table 248

Compounds of the formula I in which R^{a} is CF₃, R^{b2} is C¹, R^{b3} is F and R^{b4} is F and the combination of R¹, R³, R⁹ and R¹⁰ for a compound corresponds in each case to one row of table A

Table 249

Compounds of the formula I in which R^{a} is CF₃, R^{b2} is C¹, R^{b3} is F and R^{b4} is Cl and the combination of R¹, R³, R⁹ and R¹⁰ for a compound corresponds in each case to one row of table A

Table 250

Compounds of the formula I in which R^{a} is CF₃, R^{b2} is C¹, R^{b3} is F and R^{b4} is Br and the combination of R¹, R³, R⁹ and R¹⁰ for a compound corresponds in each case to one row of table A

Table 251

Compounds of the formula I in which R^{a} is CF₃, R^{b2} is Cl, R^{b3} is F and R^{b4} is CH₃ and the combination of R¹, R³, R⁹ and R¹⁰ for a compound corresponds in each case to one row of table A

Table 252

Compounds of the formula I in which R^{a} is CF₃, R^{b2} is C¹, R^{b3} is F and R^{b4} is OCH₃ and the combination of R¹, R³, R⁹ and R¹⁰ for a compound corresponds in each case to one row of table A

Table 253

Compounds of the formula I in which R^{a} is OCH₃, R^{b2} is C¹, R^{b3} is H and R^{b4} is H and the combination of R¹, R³, R⁹ and R¹⁰ for a compound corresponds in each case to one row of table A

Table 254

Compounds of the formula I in which R^{a} is OCH₃, R^{b2} is C¹, R^{b3} is H and R^{b4} is F and the combination of R¹, R³, R⁹ and R¹⁰ for a compound corresponds in each case to one row of table A

Table 255

Compounds of the formula I in which R^{a} is OCH₃, R^{b2} is C¹, R^{b3} is H and R^{b4} is Cl and the combination of R¹, R³, R⁹ and R¹⁰ for a compound corresponds in each case to one row of table A

Table 256

Compounds of the formula I in which R^{a} is OCH₃, R^{b2} is C¹, R^{b3} is H and R^{b4} is Br and the combination of R¹, R³, R⁹ and R¹⁰ for a compound corresponds in each case to one row of table A

Table 257

Compounds of the formula I in which R^{a} is OCH₃, R^{b2} is C¹, R^{b3} is H and R^{b4} is CH₃ and the combination of R¹, R³, R⁹ and R¹⁰ for a compound corresponds in each case to one row of table A

Table 258

Compounds of the formula I in which R^{a} is OCH₃, R^{b2} is C¹, R^{b3} is H and R^{b4} is OCH₃ and the combination of R¹, R³, R⁹ and R¹⁰ for a compound corresponds in each case to one row of table A

Table 259

Compounds of the formula I in which R^{a} is OCH₃, R^{b2} is C¹, R^{b3} is F and R^{b4} is H and the combination of R¹, R³, R⁹ and R¹⁰ for a compound corresponds in each case to one row of table A

Table 260

Compounds of the formula I in which R^{a} is OCH₃, R^{b2} is C¹, R^{b3} is F and R^{b4} is F and the combination of R¹, R³, R⁹ and R¹⁰ for a compound corresponds in each case to one row of table A

Table 261

Compounds of the formula I in which R^{a} is OCH₃, R^{b2} is Cl, R^{b3} is F and R^{b4} is Cl and the combination of R¹, R³, R⁹ and R¹⁰ for a compound corresponds in each case to one row of table A

Table 262

Compounds of the formula I in which R^{a} is OCH₃, R^{b2} is Cl, R^{b3} is F and R^{b4} is Br and the combination of R¹, R³, R⁹ and R¹⁰ for a compound corresponds in each case to one row of table A

Table 263

Compounds of the formula I in which R^{a} is OCH₃, R^{b2} is Cl, R^{b3} is F and R^{b4} is CH₃ and the combination of R¹, R³, R⁹ and R¹⁰ for a compound corresponds in each case to one row of table A

Table 264

Compounds of the formula I in which R^{a} is OCH₃, R^{b2} is Cl, R^{b3} is F and R^{b4} is OCH₃ and the combination of R¹, R³, R⁹ and R¹⁰ for a compound corresponds in each case to one row of table A

Table 265

Compounds of the formula I in which R^{a} is OC₂H₅, R^{b2} is Cl, R^{b3} is H and R^{b4} is H and the combination of R¹, R³, R⁹ and R¹⁰ for a compound corresponds in each case to one row of table A

Table 266

Compounds of the formula I in which R^{a} is OC₂H₅, R^{b2} is Cl, R^{b3} is H and R^{b4} is F and the combination of R¹, R³, R⁹ and R¹⁰ for a compound corresponds in each case to one row of table A

Table 267

Compounds of the formula I in which R^{a} is OC₂H₅, R^{b2} is Cl, R^{b3} is H and R^{b4} is Cl and the combination of R¹, R³, R⁹ and R¹⁰ for a compound corresponds in each case to one row of table A

Table 268

Compounds of the formula I in which R^{a} is OC₂H₅, R^{b2} is Cl, R^{b3} is H and R^{b4} is Br and the combination of R¹, R³, R⁹ and R¹⁰ for a compound corresponds in each case to one row of table A

Table 269

Compounds of the formula I in which R^{a} is OC₂H₅, R^{b2} is Cl, R^{b3} is H and R^{b4} is CH₃ and the combination of R¹, R³, R⁹ and R¹⁰ for a compound corresponds in each case to one row of table A

Table 270

Compounds of the formula I in which R^{a} is OC₂H₅, R^{b2} is Cl, R^{b3} is H and R^{b4} is OCH₃ and the combination of R¹, R³, R⁹ and R¹⁰ for a compound corresponds in each case to one row of table A

Table 271

Compounds of the formula I in which R^{a} is OC₂H₅, R^{b2} is Cl, R^{b3} is F and R^{b4} is H and the combination of R¹, R³, R⁹ and R¹⁰ for a compound corresponds in each case to one row of table A

Table 272

Compounds of the formula I in which R^{a} is OC₂H₅, R^{b2} is Cl, R^{b3} is F and R^{b4} is F and the combination of R¹, R³, R⁹ and R¹⁰ for a compound corresponds in each case to one row of table A

Table 273

Compounds of the formula I in which R^{a} is OC₂H₅, R^{b2} is Cl, R^{b3} is F and R^{b4} is Cl and the combination of R¹, R³, R⁹ and R¹⁰ for a compound corresponds in each case to one row of table A

Table 274

Compounds of the formula I in which R^{a} is OC₂H₅, R^{b2} is Cl, R^{b3} is F and R^{b4} is Br and the combination of R¹, R³, R⁹ and R¹⁰ for a compound corresponds in each case to one row of table A

Table 275

Compounds of the formula I in which R^{a} is OC₂H₅, R^{b2} is Cl, R^{b3} is F and R^{b4} is CH₃ and the combination of R¹, R³, R⁹ and R¹⁰ for a compound corresponds in each case to one row of table A

Table 276

Compounds of the formula I in which R^{a} is OC₂H₅, R^{b2} is Cl, R^{b3} is F and R^{b4} is OCH₃ and the combination of R¹, R³, R⁹ and R¹⁰ for a compound corresponds in each case to one row of table A

Table 277

Compounds of the formula I in which R^{a} is morpholin-1-yl, R^{b2} is Cl and R^{b3} is H and R^{b4} is H and the combination of R¹, R³, R⁹ and R¹⁰ for a compound corresponds in each case to one row of table A

Table 278

Compounds of the formula I in which R^{a} is morpholin-1-yl, R^{b2} is Cl and R^{b3} is H and R^{b4} is F and the combination of R¹, R³, R⁹ and R¹⁰ for a compound corresponds in each case to one row of table A

Table 279

Compounds of the formula I in which R^{a} is morpholin-1-yl, R^{b2} is Cl and R^{b3} is H and R^{b4} is Cl and the combination of R¹, R³, R⁹ and R¹⁰ for a compound corresponds in each case to one row of table A

Table 280

Compounds of the formula I in which R^{a} is morpholin-1-yl, R^{b2} is Cl and R^{b3} is H and R^{b4} is Br and the combination of R¹, R³, R⁹ and R¹⁰ for a compound corresponds in each case to one row of table A

Table 281

Compounds of the formula I in which R^{a} is morpholin-1-yl, R^{b2} is Cl and R^{b3} is H and R^{b4} is CH₃ and the combination of R¹, R³, R⁹ and R¹⁰ for a compound corresponds in each case to one row of table A

Table 282

Compounds of the formula I in which R^{a} is morpholin-1-yl, R^{b2} is Cl and R^{b3} is H and R^{b4} is OCH₃ and the combination of R¹, R³, R⁹ and R¹⁰ for a compound corresponds in each case to one row of table A

Table 283

Compounds of the formula I in which R^{a} is morpholin-1-yl, R^{b2} is Cl, R^{b3} is F and R^{b4} is H and the combination of R¹, R³, R⁹ and R¹⁰ for a compound corresponds in each case to one row of table A

Table 284

Compounds of the formula I in which R^{a} is morpholin-1-yl, R^{b2} is Cl, R^{b3} is F and R^{b4} is F and the combination of R¹, R³, R⁹ and R¹⁰ for a compound corresponds in each case to one row of table A

Table 285

Compounds of the formula I in which R^{a} is morpholin-1-yl, R^{b2} is Cl, R^{b3} is F and R^{b4} is Cl and the combination of R¹, R³, R⁹ and R¹⁰ for a compound corresponds in each case to one row of table A

Table 286

Compounds of the formula I in which R^{a} is morpholin-1-yl, R^{b2} is Cl, R^{b3} is F and R^{b4} is Br and the combination of R¹, R³, R⁹ and R¹⁰ for a compound corresponds in each case to one row of table A

Table 287

Compounds of the formula I in which R^{a} is morpholin-1-yl, R^{b2} is Cl, R^{b3} is F and R^{b4} is CH₃ and the combination of R¹, R³, R⁹ and R¹⁰ for a compound corresponds in each case to one row of table A

Table 288

Compounds of the formula I in which R^{a} is morpholin-1-yl, R^{b2} is Cl, R^{b3} is F and R^{b4} is OCH₃ and the combination of R¹, R³, R⁹ and R¹⁰ for a compound corresponds in each case to one row of table A

Table 289

Compounds of the formula I in which R^{a} is CN, R^{b2} is CH₃, R^{b3} and R^{b4} are H and the combination of R¹, R³, R⁹ and R¹⁰ for a compound corresponds in each case to one row of table A

Table 290

Compounds of the formula I in which R^{a} is CN, R^{b2} is CH₃, R^{b3} is H and R^{b4} is F and the combination of R¹, R³, R⁹ and R¹⁰ for a compound corresponds in each case to one row of table A

Table 291

Compounds of the formula I in which R^{a} is CN, R^{b2} is CH₃, R^{b3} is H and R^{b4} is Cl and the combination of R¹, R³, R⁹ and R¹⁰ for a compound corresponds in each case to one row of table A

Table 292

Compounds of the formula I in which R^{a} is CN, R^{b2} is CH₃, R^{b3} is H and R^{b4} is Br and the combination of R¹, R³, R⁹ and R¹⁰ for a compound corresponds in each case to one row of table A

Table 293

Compounds of the formula I in which R^{a} is CN, R^{b2} is CH₃, R^{b3} is H and R^{b4} is CH₃ and the combination of R¹, R³, R⁹ and R¹⁰ for a compound corresponds in each case to one row of table A

Table 294

Compounds of the formula I in which R^{a} is CN, R^{b2} is CH₃, R^{b3} is H and R^{b4} is OCH₃ and the combination of R¹, R³, R⁹ and R¹⁰ for a compound corresponds in each case to one row of table A

Table 295

Compounds of the formula I in which R^{a} is CN, R^{b2} is CH₃, R^{b3} is F and R^{b4} is H and the combination of R¹, R³, R⁹ and R¹⁰ for a compound corresponds in each case to one row of table A

Table 296

Compounds of the formula I in which R^{a} is CN, R^{b2} is CH₃, R^{b3} and R^{b4} are F and the combination of R¹, R³, R⁹ and R¹⁰ for a compound corresponds in each case to one row of table A

Table 297

Compounds of the formula I in which R^{a} is CN, R^{b2} is CH₃, R^{b3} is F and R^{b4} is Cl and the combination of R¹, R³, R⁹ and R¹⁰ for a compound corresponds in each case to one row of table A

Table 298

Compounds of the formula I in which R^{a} is CN, R^{b2} is CH₃, R^{b3} is F and R^{b4} is Br and the combination of R¹, R³, R⁹ and R¹⁰ for a compound corresponds in each case to one row of table A

Table 299

Compounds of the formula I in which R^{a} is CN, R^{b2} is CH₃, R^{b3} is F and R^{b4} is CH₃ and the combination of R¹, R³, R⁹ and R¹⁰ for a compound corresponds in each case to one row of table A

Table 300

Compounds of the formula I in which R^{a} is CN, R^{b2} is CH₃, R^{b3} is F and R^{b4} is OCH₃ and the combination of R¹, R³, R⁹ and R¹⁰ for a compound corresponds in each case to one row of table A

Table 301

Compounds of the formula I in which R^{a} is C¹, R^{b2} is CH₃, R^{b3} and R^{b4} are H and the combination of R¹, R³, R⁹ and R¹⁰ for a compound corresponds in each case to one row of table A

Table 302

Compounds of the formula I in which R^{a} is C¹, R^{b2} is CH₃, R^{b3} is H and R^{b4} is F and the combination of R¹, R³, R⁹ and R¹⁰ for a compound corresponds in each case to one row of table A

Table 303

Compounds of the formula I in which R^{a} is C¹, R^{b2} is CH₃, R^{b3} is H and R^{b4} is Cl and the combination of R¹, R³, R⁹ and R¹⁰ for a compound corresponds in each case to one row of table A

Table 304

Compounds of the formula I in which R^{a} is C¹, R^{b2} is CH₃, R^{b3} is H and R^{b4} is Br and the combination of R¹, R³, R⁹ and R¹⁰ for a compound corresponds in each case to one row of table A

Table 305

Compounds of the formula I in which R^{a} is C¹, R^{b2} is CH₃, R^{b3} is H and R^{b4} is CH₃ and the combination of R¹, R³, R⁹ and R¹⁰ for a compound corresponds in each case to one row of table A

Table 306

Compounds of the formula I in which R^{a} is C¹, R^{b2} is CH₃, R^{b3} is H and R^{b4} is OCH₃ and the combination of R¹, R³, R⁹ and R¹⁰ for a compound corresponds in each case to one row of table A

Table 307

Compounds of the formula I in which R^{a} is C¹, R^{b2} is CH₃, R^{b3} is F and R^{b4} is H and the combination of R¹, R³, R⁹ and R¹⁰ for a compound corresponds in each case to one row of table A

Table 308

Compounds of the formula I in which R^{a} is C¹, R^{b2} is CH₃, R^{b3} and R^{b4} are F and the combination of R¹, R³, R⁹ and R¹⁰ for a compound corresponds in each case to one row of table A

Table 309

Compounds of the formula I in which R^{a} is C¹, R^{b2} is CH₃, R^{b3} is F and R^{b4} is Cl and the combination of R¹, R³, R⁹ and R¹⁰ for a compound corresponds in each case to one row of table A

Table 310

Compounds of the formula I in which R^{a} is C¹, R^{b2} is CH₃, R^{b3} is F and R^{b4} is Br and the combination of R¹, R³, R⁹ and R¹⁰ for a compound corresponds in each case to one row of table A

Table 311

Compounds of the formula I in which R^{a} is C¹, R^{b2} is CH₃, R^{b3} is F and R^{b4} is CH₃ and the combination of R¹, R³, R⁹ and R¹⁰ for a compound corresponds in each case to one row of table A

Table 312

Compounds of the formula I in which R^{a} is C¹, R^{b2} is CH₃, R^{b3} is F and R^{b4} is OCH₃ and the combination of R¹, R³, R⁹ and R¹⁰ for a compound corresponds in each case to one row of table A

Table 313

Compounds of the formula I in which R^{a} is NO₂, R^{b2} is CH₃, R^{b3} and R^{b4} are H and the combination of R¹, R³, R⁹ and R¹⁰ for a compound corresponds in each case to one row of table A

Table 314

Compounds of the formula I in which R^{a} is NO₂, R^{b2} is CH₃, R^{b3} is H and R^{b4} is F and the combination of R¹, R³, R⁹ and R¹⁰ for a compound corresponds in each case to one row of table A

Table 315

Compounds of the formula I in which R^{a} is NO₂, R^{b2} is CH₃, R^{b3} is H and R^{b4} is Cl and the combination of R¹, R³, R⁹ and R¹⁰ for a compound corresponds in each case to one row of table A

Table 316

Compounds of the formula I in which R^{a} is NO₂, R^{b2} is CH₃, R^{b3} is H and R^{b4} is Br and the combination of R¹, R³, R⁹ and R¹⁰ for a compound corresponds in each case to one row of table A

Table 317

Compounds of the formula I in which R^{a} is NO₂, R^{b2} is CH₃, R^{b3} is H and R^{b4} is CH₃ and the combination of R¹, R³, R⁹ and R¹⁰ for a compound corresponds in each case to one row of table A

Table 318

Compounds of the formula I in which R^{a} is NO₂, R^{b2} is CH₃, R^{b3} is H and R^{b4} is OCH₃ and the combination of R¹, R³, R⁹ and R¹⁰ for a compound corresponds in each case to one row of table A

Table 319

Compounds of the formula I in which R^{a} is NO₂, R^{b2} is CH₃, R^{b3} is F and R^{b4} is H and the combination of R¹, R³, R⁹ and R¹⁰ for a compound corresponds in each case to one row of table A

Table 320

Compounds of the formula I in which R^{a} is NO₂, R^{b2} is CH₃, R^{b3} and R^{b4} are F and the combination of R¹, R³, R⁹ and R¹⁰ for a compound corresponds in each case to one row of table A

Table 321

Compounds of the formula I in which R^{a} is NO₂, R^{b2} is CH₃, R^{b3} is F and R^{b4} is Cl and the combination of R¹, R³, R⁹ and R¹⁰ for a compound corresponds in each case to one row of table A

Table 322

Compounds of the formula I in which R^{a} is NO₂, R^{b2} is CH₃, R^{b3} is F and R^{b4} is Br and the combination of R¹, R³, R⁹ and R¹⁰ for a compound corresponds in each case to one row of table A

Table 323

Compounds of the formula I in which R^{a} is NO₂, R^{b2} is CH₃, R^{b3} is F and R^{b4} is CH₃ and the combination of R¹, R³, R⁹ and R¹⁰ for a compound corresponds in each case to one row of table A

Table 324

Compounds of the formula I in which R^{a} is NO₂, R^{b2} is CH₃, R^{b3} is F and R^{b4} is OCH₃ and the combination of R¹, R³, R⁹ and R¹⁰ for a compound corresponds in each case to one row of table A

Table 325

Compounds of the formula I in which R^{a} is CH₃, R^{b2} is CH₃, R^{b3} is H and R^{b4} is H and the combination of R¹, R³, R⁹ and R¹⁰ for a compound corresponds in each case to one row of table A

Table 326

Compounds of the formula I in which R^{a} is CH₃, R^{b2} is CH₃, R^{b3} is H and R^{b4} is F and the combination of R¹, R³, R⁹ and R¹⁰ for a compound corresponds in each case to one row of table A

Table 327

Compounds of the formula I in which R^{a} is CH₃, R^{b2} is CH₃, R^{b3} is H and R^{b4} is Cl and the combination of R¹, R³, R⁹ and R¹⁰ for a compound corresponds in each case to one row of table A

Table 328

Compounds of the formula I in which R^{a} is CH₃, R^{b2} is CH₃, R^{b3} is H and R^{b4} is Br and the combination of R¹, R³, R⁹ and R¹⁰ for a compound corresponds in each case to one row of table A

Table 329

Compounds of the formula I in which R^{a} is CH₃, R^{b2} is CH₃, R^{b3} is H and R^{b4} is CH₃ and the combination of R¹, R³, R⁹ and R¹⁰ for a compound corresponds in each case to one row of table A

Table 330

Compounds of the formula I in which R^{a} is CH₃, R^{b2} is CH₃, R^{b3} is H and R^{b4} is OCH₃ and the combination of R¹, R³, R⁹ and R¹⁰ for a compound corresponds in each case to one row of table A

Table 331

Compounds of the formula I in which R^{a} is CH₃, R^{b2} is CH₃, R^{b3} is F and R^{b4} is H and the combination of R¹, R³, R⁹ and R¹⁰ for a compound corresponds in each case to one row of table A

Table 332

Compounds of the formula I in which R^{a} is CH₃, R^{b2} is CH₃, R^{b3} is F and R^{b4} is F and the combination of R¹, R³, R⁹ and R¹⁰ for a compound corresponds in each case to one row of table A

Table 333

Compounds of the formula I in which R^{a} is CH₃, R^{b2} is CH₃, R^{b3} is F and R^{b4} is Cl and the combination of R¹, R³, R⁹ and R¹⁰ for a compound corresponds in each case to one row of table A

Table 334

Compounds of the formula I in which R^{a} is CH₃, R^{b2} is CH₃, R^{b3} is F and R^{b4} is Br and the combination of R¹, R³, R⁹ and R¹⁰ for a compound corresponds in each case to one row of table A

Table 335

Compounds of the formula I in which R^{a} is CH₃, R^{b2} is CH₃, R^{b3} is F and R^{b4} is CH₃ and the combination of R¹, R³, R⁹ and R¹⁰ for a compound corresponds in each case to one row of table A

Table 336

Compounds of the formula I in which R^{a} is CH₃, R^{b2} is CH₃, R^{b3} is F and R^{b4} is OCH₃ and the combination of R¹, R³, R⁹ and R¹⁰ for a compound corresponds in each case to one row of table A

Table 337

Compounds of the formula I in which R^{a} is CF₃, R^{b2} is CH₃, R^{b3} is H and R^{b4} is H and the combination of R¹, R³, R⁹ and R¹⁰ for a compound corresponds in each case to one row of table A

Table 338

Compounds of the formula I in which R^{a} is CF₃, R^{b2} is CH₃, R^{b3} is H and R^{b4} is F and the combination of R¹, R³, R⁹ and R¹⁰ for a compound corresponds in each case to one row of table A

Table 339

Compounds of the formula I in which R^{a} is CF₃, R^{b2} is CH₃, R^{b3} is H and R^{b4} is Cl and the combination of R¹, R³, R⁹ and R¹⁰ for a compound corresponds in each case to one row of table A

Table 340

Compounds of the formula I in which R^{a} is CF₃, R^{b2} is CH₃, R^{b3} is H and R^{b4} is Br and the combination of R¹, R³, R⁹ and R¹⁰ for a compound corresponds in each case to one row of table A

Table 341

Compounds of the formula I in which R^{a} is CF₃, R^{b2} is CH₃, R^{b3} is H and R^{b4} is CH₃ and the combination of R¹, R³, R⁹ and R¹⁰ for a compound corresponds in each case to one row of table A

Table 342

Compounds of the formula I in which R^{a} is CF₃, R^{b2} is CH₃, R^{b3} is H and R^{b4} is OCH₃ and the combination of R¹, R³, R⁹ and R¹⁰ for a compound corresponds in each case to one row of table A

Table 343

Compounds of the formula I in which R^{a} is CF₃, R^{b2} is CH₃, R^{b3} is F and R^{b4} is H and the combination of R¹, R³, R⁹ and R¹⁰ for a compound corresponds in each case to one row of table A

Table 344

Compounds of the formula I in which R^{a} is CF₃, R^{b2} is CH₃, R^{b3} is F and R^{b4} is F and the combination of R¹, R³, R⁹ and R¹⁰ for a compound corresponds in each case to one row of table A

Table 345

Compounds of the formula I in which R^{a} is CF₃, R^{b2} is CH₃, R^{b3} is F and R^{b4} is Cl and the combination of R¹, R³, R⁹ and R¹⁰ for a compound corresponds in each case to one row of table A

Table 346

Compounds of the formula I in which R^{a} is CF₃, R^{b2} is CH₃, R^{b3} is F and R^{b4} is Br and the combination of R¹, R³, R⁹ and R¹⁰ for a compound corresponds in each case to one row of table A

Table 347

Compounds of the formula I in which R^{a} is CF₃, R^{b2} is CH₃, R^{b3} is F and R^{b4} is CH₃ and the combination of R¹, R³, R⁹ and R¹⁰ for a compound corresponds in each case to one row of table A

Table 348

Compounds of the formula I in which R^{a} is CF₃, R^{b2} is CH₃, R^{b3} is F and R^{b4} is OCH₃ and the combination of R¹, R³, R⁹ and R¹⁰ for a compound corresponds in each case to one row of table A

Table 349

Compounds of the formula I in which R^{a} is OCH₃, R^{b2} is CH₃, R^{b3} is H and R^{b4} is H and the combination of R¹, R³, R⁹ and R¹⁰ for a compound corresponds in each case to one row of table A

Table 350

Compounds of the formula I in which R^{a} is OCH₃, R^{b2} is CH₃, R^{b3} is H and R^{b4} is F and the combination of R¹, R³, R⁹ and R¹⁰ for a compound corresponds in each case to one row of table A

Table 351

Compounds of the formula I in which R^{a} is OCH₃, R^{b2} is CH₃, R^{b3} is H and R^{b4} is Cl and the combination of R¹, R³, R⁹ and R¹⁰ for a compound corresponds in each case to one row of table A

Table 352

Compounds of the formula I in which R^{a} is OCH₃, R^{b2} is CH₃, R^{b3} is H and R^{b4} is Br and the combination of R¹, R³, R⁹ and R¹⁰ for a compound corresponds in each case to one row of table A

Table 353

Compounds of the formula I in which R^{a} is OCH₃, R^{b2} is CH₃, R^{b3} is H and R^{b4} is CH₃ and the combination of R¹, R³, R⁹ and R¹⁰ for a compound corresponds in each case to one row of table A

Table 354

Compounds of the formula I in which R^{a} is OCH₃, R^{b2} is CH₃, R^{b3} is H and R^{b4} is OCH₃ and the combination of R¹, R³, R⁹ and R¹⁰ for a compound corresponds in each case to one row of table A

Table 355

Compounds of the formula I in which R^{a} is OCH₃, R^{b2} is CH₃, R^{b3} is F and R^{b4} is H and the combination of R¹, R³, R⁹ and R¹⁰ for a compound corresponds in each case to one row of table A

Table 356

Compounds of the formula I in which R^{a} is OCH₃, R^{b2} is CH₃, R^{b3} is F and R^{b4} is F and the combination of R¹, R³, R⁹ and R¹⁰ for a compound corresponds in each case to one row of table A

Table 357

Compounds of the formula I in which R^{a} is OCH₃, R^{b2} is CH₃, R^{b3} is F and R^{b4} is Cl and the combination of R¹, R³, R⁹ and R¹⁰ for a compound corresponds in each case to one row of table A

Table 358

Compounds of the formula I in which R^{a} is OCH₃, R^{b2} is CH₃, R^{b3} is F and R^{b4} is Br and the combination of R¹, R³, R⁹ and R¹⁰ for a compound corresponds in each case to one row of table A

Table 359

Compounds of the formula I in which R^{a} is OCH₃, R^{b2} is CH₃, R^{b3} is F and R^{b4} is CH₃ and the combination of R¹, R³, R⁹ and R¹⁰ for a compound corresponds in each case to one row of table A

Table 360

Compounds of the formula I in which R^{a} is OCH₃, R^{b2} is CH₃, R^{b3} is F and R^{b4} is OCH₃ and the combination of R¹, R³, R⁹ and R¹⁰ for a compound corresponds in each case to one row of table A

Table 361

Compounds of the formula I in which R^{a} is OC₂H₅, R^{b2} is CH₃, R^{b3} is H and R^{b4} is H and the combination of R¹, R³, R⁹ and R¹⁰ for a compound corresponds in each case to one row of table A

Table 362

Compounds of the formula I in which R^{a} is OC₂H₅, R^{b2} is CH₃, R^{b3} is H and R^{b4} is F and the combination of R¹, R³, R⁹ and R¹⁰ for a compound corresponds in each case to one row of table A

Table 363

Compounds of the formula I in which R^{a} is OC₂H₅, R^{b2} is CH₃, R^{b3} is H and R^{b4} is Cl and the combination of R¹, R³, R⁹ and R¹⁰ for a compound corresponds in each case to one row of table A

Table 364

Compounds of the formula I in which R^{a} is OC₂H₅, R^{b2} is CH₃, R^{b3} is H and R^{b4} is Br and the combination of R¹, R³, R⁹ and R¹⁰ for a compound corresponds in each case to one row of table A

Table 365

Compounds of the formula I in which R^{a} is OC₂H₅, R^{b2} is CH₃, R^{b3} is H and R^{b4} is CH₃ and the combination of R¹, R³, R⁹ and R¹⁰ for a compound corresponds in each case to one row of table A

Table 366

Compounds of the formula I in which R^{a} is OC₂H₅, R^{b2} is CH₃, R^{b3} is H and R^{b4} is OCH₃ and the combination of R¹, R³, R⁹ and R¹⁰ for a compound corresponds in each case to one row of table A

Table 367

Compounds of the formula I in which R^{a} is OC₂H₅, R^{b2} is CH₃, R^{b3} is F and R^{b4} is H and the combination of R¹, R³, R⁹ and R¹⁰ for a compound corresponds in each case to one row of table A

Table 368

Compounds of the formula I in which R^{a} is OC₂H₅, R^{b2} is CH₃, R^{b3} is F and R^{b4} is F and the combination of R¹, R³, R⁹ and R¹⁰ for a compound corresponds in each case to one row of table A

Table 369

Compounds of the formula I in which R^{a} is OC₂H₅, R^{b2} is CH₃, R^{b3} is F and R^{b4} is Cl and the combination of R¹, R³, R⁹ and R¹⁰ for a compound corresponds in each case to one row of table A

Table 370

Compounds of the formula I in which R^{a} is OC₂H₅, R^{b2} is CH₃, R^{b3} is F and R^{b4} is Br and the combination of R¹, R³, R⁹ and R¹⁰ for a compound corresponds in each case to one row of table A

Table 371

Compounds of the formula I in which R^{a} is OC₂H₅, R^{b2} is CH₃, R^{b3} is F and R^{b4} is CH₃ and the combination of R¹, R³, R⁹ and R¹⁰ for a compound corresponds in each case to one row of table A

Table 372

Compounds of the formula I in which R^{a} is OC₂H₅, R^{b2} is CH₃, R^{b3} is F and R^{b4} is OCH₃ and the combination of R¹, R³, R⁹ and R¹⁰ for a compound corresponds in each case to one row of table A

Table 373

Compounds of the formula I in which R^{a} is morpholin-1-yl, R^{b2} is CH₃ and R^{b3} is H and R^{b4} is H and the combination of R¹, R³, R⁹ and R¹⁰ for a compound corresponds in each case to one row of table A

Table 374

Compounds of the formula I in which R^{a} is morpholin-1-yl, R^{b2} is CH₃ and R^{b3} is H and R^{b4} is F and the combination of R¹, R³, R⁹ and R¹⁰ for a compound corresponds in each case to one row of table A

Table 375

Compounds of the formula I in which R^{a} is morpholin-1-yl, R^{b2} is CH₃ and R^{b3} is H and R^{b4} is Cl and the combination of R¹, R³, R⁹ and R¹⁰ for a compound corresponds in each case to one row of table A

Table 376

Compounds of the formula I in which R^{a} is morpholin-1-yl, R^{b2} is CH₃ and R^{b3} is H and R^{b4} is Br and the combination of R¹, R³, R⁹ and R¹⁰ for a compound corresponds in each case to one row of table A

Table 377

Compounds of the formula I in which R^{a} is morpholin-1-yl, R^{b2} is CH₃ and R^{b3} is H and R^{b4} is CH₃ and the combination of R¹, R³, R⁹ and R¹⁰ for a compound corresponds in each case to one row of table A

Table 378

Compounds of the formula I in which R^{a} is morpholin-1-yl, R^{b2} is CH₃ and R^{b3} is H and R^{b4} is OCH₃ and the combination of R¹, R³, R⁹ and R¹⁰ for a compound corresponds in each case to one row of table A

Table 379

Compounds of the formula I in which R^{a} is morpholin-1-yl, R^{b2} is CH₃, R^{b3} is F and R^{b4} is H and the combination of R¹, R³, R⁹ and R¹⁰ for a compound corresponds in each case to one row of table A

Table 380

Compounds of the formula I in which R^{a} is morpholin-1-yl, R^{b2} is CH₃, R^{b3} is F and R^{b4} is F and the combination of R¹, R³, R⁹ and R¹⁰ for a compound corresponds in each case to one row of table A

Table 381

Compounds of the formula I in which R^{a} is morpholin-1-yl, R^{b2} is CH₃, R^{b3} is F and R^{b4} is Cl and the combination of R¹, R³, R⁹ and R¹⁰ for a compound corresponds in each case to one row of table A

Table 382

Compounds of the formula I in which R^{a} is morpholin-1-yl, R^{b2} is CH₃, R^{b3} is F and R^{b4} is Br and the combination of R¹, R³, R⁹ and R¹⁰ for a compound corresponds in each case to one row of table A

Table 383

Compounds of the formula I in which R^{a} is morpholin-1-yl, R^{b2} is CH₃, R^{b3} is F and R^{b4} is CH₃ and the combination of R¹, R³, R⁹ and R¹⁰ for a compound corresponds in each case to one row of table A

Table 384

Compounds of the formula I in which R^{a} is morpholin-1-yl, R^{b2} is CH₃, R^{b3} is F and R^{b4} is OCH₃ and the combination of R¹, R³, R⁹ and R¹⁰ for a compound corresponds in each case to one row of table A

Table 385

Compounds of the formula I in which R^{a} is CN, R^{b2} is OCH₃, R^{b3} and R^{b4} are H and the combination of R¹, R³, R⁹ and R¹⁰ for a compound corresponds in each case to one row of table A

Table 386

Compounds of the formula I in which R^{a} is CN, R^{b2} is OCH₃, R^{b3} is H and R^{b4} is F and the combination of R¹, R³, R⁹ and R¹⁰ for a compound corresponds in each case to one row of table A

Table 387

Compounds of the formula I in which R^{a} is CN, R^{b2} is OCH₃, R^{b3} is H and R^{b4} is Cl and the combination of R¹, R³, R⁹ and R¹⁰ for a compound corresponds in each case to one row of table A

Table 388

Compounds of the formula I in which R^{a} is CN, R^{b2} is OCH₃, R^{b3} is H and R^{b4} is Br and the combination of R¹, R³, R⁹ and R¹⁰ for a compound corresponds in each case to one row of table A

Table 389

Compounds of the formula I in which R^{a} is CN, R^{b2} is OCH₃, R^{b3} is H and R^{b4} is CH₃ and the combination of R¹, R³, R⁹ and R¹⁰ for a compound corresponds in each case to one row of table A

Table 390

Compounds of the formula I in which R^{a} is CN, R^{b2} is OCH₃, R^{b3} is H and R^{b4} is OCH₃ and the combination of R¹, R³, R⁹ and R¹⁰ for a compound corresponds in each case to one row of table A

Table 391

Compounds of the formula I in which R^{a} is CN, R^{b2} is OCH₃, R^{b3} is F and R^{b4} is H and the combination of R¹, R³, R⁹ and R¹⁰ for a compound corresponds in each case to one row of table A

Table 392

Compounds of the formula I in which R^{a} is CN, R^{b2} is OCH₃, R^{b3} and R^{b4} are F and the combination of R¹, R³, R⁹ and R¹⁰ for a compound corresponds in each case to one row of table A

Table 393

Compounds of the formula I in which R^{a} is CN, R^{b2} is OCH₃, R^{b3} is F and R^{b4} is Cl and the combination of R¹, R³, R⁹ and R¹⁰ for a compound corresponds in each case to one row of table A

Table 394

Compounds of the formula I in which R^{a} is CN, R^{b2} is OCH₃, R^{b3} is F and R^{b4} is Br and the combination of R¹, R³, R⁹ and R¹⁰ for a compound corresponds in each case to one row of table A

Table 395

Compounds of the formula I in which R^{a} is CN, R^{b2} is OCH₃, R^{b3} is F and R^{b4} is CH₃ and the combination of R¹, R³, R⁹ and R¹⁰ for a compound corresponds in each case to one row of table A

Table 396

Compounds of the formula I in which R^{a} is CN, R^{b2} is OCH₃, R^{b3} is F and R^{b4} is OCH₃ and the combination of R¹, R³, R⁹ and R¹⁰ for a compound corresponds in each case to one row of table A

Table 397

Compounds of the formula I in which R^{a} is Cl, R^{b2} is OCH₃, R^{b3} and R^{b4} are H and the combination of R¹, R³, R⁹ and R¹⁰ for a compound corresponds in each case to one row of table A

Table 398

Compounds of the formula I in which R^{a} is Cl, R^{b2} is OCH₃, R^{b3} is H and R^{b4} is F and the combination of R¹, R³, R⁹ and R¹⁰ for a compound corresponds in each case to one row of table A

Table 399

Compounds of the formula I in which R^{a} is Cl, R^{b2} is OCH₃, R^{b3} is H and R^{b4} is Cl and the combination of R¹, R³, R⁹ and R¹⁰ for a compound corresponds in each case to one row of table A

Table 400

Compounds of the formula I in which R^{a} is Cl, R^{b2} is OCH₃, R^{b3} is H and R^{b4} is Br and the combination of R¹, R³, R⁹ and R¹⁰ for a compound corresponds in each case to one row of table A

Table 401

Compounds of the formula I in which R^{a} is Cl, R^{b2} is OCH₃, R^{b3} is H and R^{b4} is CH₃ and the combination of R¹, R³, R⁹ and R¹⁰ for a compound corresponds in each case to one row of table A

Table 402

Compounds of the formula I in which R^{a} is Cl, R^{b2} is OCH₃, R^{b3} is H and R^{b4} is OCH₃ and the combination of R¹, R³, R⁹ and R¹⁰ for a compound corresponds in each case to one row of table A

Table 403

Compounds of the formula I in which R^{a} is Cl, R^{b2} is OCH₃, R^{b3} is F and R^{b4} is H and the combination of R¹, R³, R⁹ and R¹⁰ for a compound corresponds in each case to one row of table A

Table 404

Compounds of the formula I in which R^{a} is Cl, R^{b2} is OCH₃, R^{b3} and R^{b4} are F and the combination of R¹, R³, R⁹ and R¹⁰ for a compound corresponds in each case to one row of table A

Table 405

Compounds of the formula I in which R^{a} is Cl, R^{b2} is OCH₃, R^{b3} is F and R^{b4} is Cl and the combination of R¹, R³, R⁹ and R¹⁰ for a compound corresponds in each case to one row of table A

Table 406

Compounds of the formula I in which R^{a} is Cl, R^{b2} is OCH₃, R^{b3} is F and R^{b4} is Br and the combination of R¹, R³, R⁹ and R¹⁰ for a compound corresponds in each case to one row of table A

Table 407

Compounds of the formula I in which R^{a} is Cl, R^{b2} is OCH₃, R^{b3} is F and R^{b4} is CH₃ and the combination of R¹, R³, R⁹ and R¹⁰ for a compound corresponds in each case to one row of table A

Table 408

Compounds of the formula I in which R^{a} is Cl, R^{b2} is OCH₃, R^{b3} is F and R^{b4} is OCH₃ and the combination of R¹, R³, R⁹ and R¹⁰ for a compound corresponds in each case to one row of table A

Table 409

Compounds of the formula I in which R^{a} is NO₂, R^{b2} is OCH₃, R^{b3} and R^{b4} are H and the combination of R¹, R³, R⁹ and R¹⁰ for a compound corresponds in each case to one row of table A

Table 410

Compounds of the formula I in which R^{a} is NO₂, R^{b2} is OCH₃, R^{b3} is H and R^{b4} is F and the combination of R¹, R³, R⁹ and R¹⁰ for a compound corresponds in each case to one row of table A

Table 411

Compounds of the formula I in which R^{a} is NO₂, R^{b2} is OCH₃, R^{b3} is H and R^{b4} is Cl and the combination of R¹, R³, R⁹ and R¹⁰ for a compound corresponds in each case to one row of table A

Table 412

Compounds of the formula I in which R^{a} is NO₂, R^{b2} is OCH₃, R^{b3} is H and R^{b4} is Br and the combination of R¹, R³, R⁹ and R¹⁰ for a compound corresponds in each case to one row of table A

Table 413

Compounds of the formula I in which R^{a} is NO₂, R^{b2} is OCH₃, R^{b3} is H and R^{b4} is CH₃ and the combination of R¹, R³, R⁹ and R¹⁰ for a compound corresponds in each case to one row of table A

Table 414

Compounds of the formula I in which R^{a} is NO₂, R^{b2} is OCH₃, R^{b3} is H and R^{b4} is OCH₃ and the combination of R¹, R³, R⁹ and R¹⁰ for a compound corresponds in each case to one row of table A

Table 415

Compounds of the formula I in which R^{a} is NO₂, R^{b2} is OCH₃, R^{b3} is F and R^{b4} is H and the combination of R¹, R³, R⁹ and R¹⁰ for a compound corresponds in each case to one row of table A

Table 416

Compounds of the formula I in which R^{a} is NO₂, R^{b2} is OCH₃, R^{b3} and R^{b4} are F and the combination of R¹, R³, R⁹ and R¹⁰ for a compound corresponds in each case to one row of table A

Table 417

Compounds of the formula I in which R^{a} is NO₂, R^{b2} is OCH₃, R^{b3} is F and R^{b4} is Cl and the combination of R¹, R³, R⁹ and R¹⁰ for a compound corresponds in each case to one row of table A

Table 418

Compounds of the formula I in which R^{a} is NO₂, R^{b2} is OCH₃, R^{b3} is F and R^{b4} is Br and the combination of R¹, R³, R⁹ and R¹⁰ for a compound corresponds in each case to one row of table A

Table 419

Compounds of the formula I in which R^{a} is NO₂, R^{b2} is OCH₃, R^{b3} is F and R^{b4} is CH₃ and the combination of R¹, R³, R⁹ and R¹⁰ for a compound corresponds in each case to one row of table A

Table 420

Compounds of the formula I in which R^{a} is NO₂, R^{b2} is OCH₃, R^{b3} is F and R^{b4} is OCH₃ and the combination of R¹, R³, R⁹ and R¹⁰ for a compound corresponds in each case to one row of table A

Table 421

Compounds of the formula I in which R^{a} is CH₃, R^{b2} is OCH₃, R^{b3} is H and R^{b4} is H and the combination of R¹, R³, R⁹ and R¹⁰ for a compound corresponds in each case to one row of table A

Table 422

Compounds of the formula I in which R^{a} is CH₃, R^{b2} is OCH₃, R^{b3} is H and R^{b4} is F and the combination of R¹, R³, R⁹ and R¹⁰ for a compound corresponds in each case to one row of table A

Table 423

Compounds of the formula I in which R^{a} is CH₃, R^{b2} is OCH₃, R^{b3} is H and R^{b4} is Cl and the combination of R¹, R³, R⁹ and R¹⁰ for a compound corresponds in each case to one row of table A

Table 424

Compounds of the formula I in which R^{a} is CH₃, R^{b2} is OCH₃, R^{b3} is H and R^{b4} is Br and the combination of R¹, R³, R⁹ and R¹⁰ for a compound corresponds in each case to one row of table A

Table 425

Compounds of the formula I in which R^{a} is CH₃, R^{b2} is OCH₃, R^{b3} is H and R^{b4} is CH₃ and the combination of R¹, R³, R⁹ and R¹⁰ for a compound corresponds in each case to one row of table A

Table 426

Compounds of the formula I in which R^{a} is CH₃, R^{b2} is OCH₃, R^{b3} is H and R^{b4} is OCH₃ and the combination of R¹, R³, R⁹ and R¹⁰ for a compound corresponds in each case to one row of table A

Table 427

Compounds of the formula I in which R^{a} is CH₃, R^{b2} is OCH₃, R^{b3} is F and R^{b4} is H and the combination of R¹, R³, R⁹ and R¹⁰ for a compound corresponds in each case to one row of table A

Table 428

Compounds of the formula I in which R^{a} is CH₃, R^{b2} is OCH₃, R^{b3} is F and R^{b4} is F and the combination of R¹, R³, R⁹ and R¹⁰ for a compound corresponds in each case to one row of table A

Table 429

Compounds of the formula I in which R^{a} is CH₃, R^{b2} is OCH₃, R^{b3} is F and R^{b4} is Cl and the combination of R¹, R³, R⁹ and R¹⁰ for a compound corresponds in each case to one row of table A

Table 430

Compounds of the formula I in which R^{a} is CH₃, R^{b2} is OCH₃, R^{b3} is F and R^{b4} is Br and the combination of R¹, R³, R⁹ and R¹⁰ for a compound corresponds in each case to one row of table A

Table 431

Compounds of the formula I in which R^{a} is CH₃, R^{b2} is OCH₃, R^{b3} is F and R^{b4} is CH₃ and the combination of R¹, R³, R⁹ and R¹⁰ for a compound corresponds in each case to one row of table A

Table 432

Compounds of the formula I in which R^{a} is CH₃, R^{b2} is OCH₃, R^{b3} is F and R^{b4} is OCH₃ and the combination of R¹, R³, R⁹ and R¹⁰ for a compound corresponds in each case to one row of table A

Table 433

Compounds of the formula I in which R^{a} is CF₃, R^{b2} is OCH₃, R^{b3} is H and R^{b4} is H and the combination of R¹, R³, R⁹ and R¹⁰ for a compound corresponds in each case to one row of table A

Table 434

Compounds of the formula I in which R^{a} is CF₃, R^{b2} is OCH₃, R^{b3} is H and R^{b4} is F and the combination of R¹, R³, R⁹ and R¹⁰ for a compound corresponds in each case to one row of table A

Table 435

Compounds of the formula I in which R^{a} is CF₃, R^{b2} is OCH₃, R^{b3} is H and R^{b4} is Cl and the combination of R¹, R³, R⁹ and R¹⁰ for a compound corresponds in each case to one row of table A

Table 436

Compounds of the formula I in which R^{a} is CF₃, R^{b2} is OCH₃, R^{b3} is H and R^{b4} is Br and the combination of R¹, R³, R⁹ and R¹⁰ for a compound corresponds in each case to one row of table A

Table 437

Compounds of the formula I in which R^{a} is CF₃, R^{b2} is OCH₃, R^{b3} is H and R^{b4} is CH₃ and the combination of R¹, R³, R⁹ and R¹⁰ for a compound corresponds in each case to one row of table A

Table 438

Compounds of the formula I in which R^{a} is CF₃, R^{b2} is OCH₃, R^{b3} is H and R^{b4} is OCH₃ and the combination of R¹, R³, R⁹ and R¹⁰ for a compound corresponds in each case to one row of table A

Table 439

Compounds of the formula I in which R^{a} is CF₃, R^{b2} is OCH₃, R^{b3} is F and R^{b4} is H and the combination of R¹, R³, R⁹ and R¹⁰ for a compound corresponds in each case to one row of table A

Table 440

Compounds of the formula I in which R^{a} is CF₃, R^{b2} is OCH₃, R^{b3} is F and R^{b4} is F and the combination of R¹, R³, R⁹ and R¹⁰ for a compound corresponds in each case to one row of table A

Table 441

Compounds of the formula I in which R^{a} is CF₃, R^{b2} is OCH₃, R^{b3} is F and R^{b4} is Cl and the combination of R¹, R³, R⁹ and R¹⁰ for a compound corresponds in each case to one row of table A

Table 442

Compounds of the formula I in which R^{a} is CF₃, R^{b2} is OCH₃, R^{b3} is F and R^{b4} is Br and the combination of R¹, R³, R⁹ and R¹⁰ for a compound corresponds in each case to one row of table A

Table 443

Compounds of the formula I in which R^{a} is CF₃, R^{b2} is OCH₃, R^{b3} is F and R^{b4} is CH₃ and the combination of R¹, R³, R⁹ and R¹⁰ for a compound corresponds in each case to one row of table A

Table 444

Compounds of the formula I in which R^{a} is CF₃, R^{b2} is OCH₃, R^{b3} is F and R^{b4} is OCH₃ and the combination of R¹, R³, R⁹ and R¹⁰ for a compound corresponds in each case to one row of table A

Table 445

Compounds of the formula I in which R^{a} is OCH₃, R^{b2} is OCH₃, R^{b3} is H and R^{b4} is H and the combination of R¹, R³, R⁹ and R¹⁰ for a compound corresponds in each case to one row of table A

Table 446

Compounds of the formula I in which R^{a} is OCH₃, R^{b2} is OCH₃, R^{b3} is H and R^{b4} is F and the combination of R¹, R³, R⁹ and R¹⁰ for a compound corresponds in each case to one row of table A

Table 447

Compounds of the formula I in which R^{a} is OCH₃, R^{b2} is OCH₃, R^{b3} is H and R^{b4} is Cl and the combination of R¹, R³, R⁹ and R¹⁰ for a compound corresponds in each case to one row of table A

Table 448

Compounds of the formula I in which R^{a} is OCH₃, R^{b2} is OCH₃, R^{b3} is H and R^{b4} is Br and the combination of R¹, R³, R⁹ and R¹⁰ for a compound corresponds in each case to one row of table A

Table 449

Compounds of the formula I in which R^{a} is OCH₃, R^{b2} is OCH₃, R^{b3} is H and R^{b4} is CH₃ and the combination of R¹, R³, R⁹ and R¹⁰ for a compound corresponds in each case to one row of table A

Table 450

Compounds of the formula I in which R^{a} is OCH₃, R^{b2} is OCH₃, R^{b3} is H and R^{b4} is OCH₃ and the combination of R¹, R³, R⁹ and R¹⁰ for a compound corresponds in each case to one row of table A

Table 451

Compounds of the formula I in which R^{a} is OCH₃, R^{b2} is OCH₃, R^{b3} is F and R^{b4} is H and the combination of R¹, R³, R⁹ and R¹⁰ for a compound corresponds in each case to one row of table A

Table 452

Compounds of the formula I in which R^{a} is OCH₃, R^{b2} is OCH₃, R^{b3} is F and R^{b4} is F and the combination of R¹, R³, R⁹ and R¹⁰ for a compound corresponds in each case to one row of table A

Table 453

Compounds of the formula I in which R^{a} is OCH₃, R^{b2} is OCH₃, R^{b3} is F and R^{b4} is C₁₋and the combination of R¹, R³, R⁹ and R¹⁰ for a compound corresponds in each case to one row of table A

Table 454

Compounds of the formula I in which R^{a} is OCH₃, R^{b2} is OCH₃, R^{b3} is F and R^{b4} is Br and the combination of R¹, R³, R⁹ and R¹⁰ for a compound corresponds in each case to one row of table A

Table 455

Compounds of the formula I in which R^{a} is OCH₃, R^{b2} is OCH₃, R^{b3} is F and R^{b4} is CH₃ and the combination of R¹, R³, R⁹ and R¹⁰ for a compound corresponds in each case to one row of table A

Table 456

Compounds of the formula I in which R^{a} is OCH₃, R^{b2} is OCH₃, R^{b3} is F and R^{b4} is OCH₃ and the combination of R¹, R³, R⁹ and R¹⁰ for a compound corresponds in each case to one row of table A

Table 457

Compounds of the formula I in which R^{a} is OC₂H₅, R^{b2} is OCH₃, R^{b3} is H and R^{b4} is H and the combination of R¹, R³, R⁹ and R¹⁰ for a compound corresponds in each case to one row of table A

Table 458

Compounds of the formula I in which R^{a} is OC₂H₅, R^{b2} is OCH₃, R^{b3} is H and R^{b4} is F and the combination of R¹, R³, R⁹ and R¹⁰ for a compound corresponds in each case to one row of table A

Table 459

Compounds of the formula I in which R^{a} is OC₂H₅, R^{b2} is OCH₃, R^{b3} is H and R^{b4} is Cl and the combination of R¹, R³, R⁹ and R¹⁰ for a compound corresponds in each case to one row of table A

Table 460

Compounds of the formula I in which R^{a} is OC₂H₅, R^{b2} is OCH₃, R^{b3} is H and R^{b4} is Br and the combination of R¹, R³, R⁹ and R¹⁰ for a compound corresponds in each case to one row of table A

Table 461

Compounds of the formula I in which R^{a} is OC₂H₅, R^{b2} is OCH₃, R^{b3} is H and R^{b4} is CH₃ and the combination of R¹, R³, R⁹ and R¹⁰ for a compound corresponds in each case to one row of table A

Table 462

Compounds of the formula I in which R^{a} is OC₂H₅, R^{b2} is OCH₃, R^{b3} is H and R^{b4} is OCH₃ and the combination of R¹, R³, R⁹ and R¹⁰ for a compound corresponds in each case to one row of table A

Table 463

Compounds of the formula I in which R^{a} is OC₂H₅, R^{b2} is OCH₃, R^{b3} is F and R^{b4} is H and the combination of R¹, R³, R⁹ and R¹⁰ for a compound corresponds in each case to one row of table A

Table 464

Compounds of the formula I in which R^{a} is OC₂H₅, R^{b2} is OCH₃, R^{b3} is F and R^{b4} is F and the combination of R¹, R³, R⁹ and R¹⁰ for a compound corresponds in each case to one row of table A

Table 465

Compounds of the formula I in which R^{a} is OC₂H₅, R^{b2} is OCH₃, R^{b3} is F and R^{b4} is Cl and the combination of R¹, R³, R⁹ and R¹⁰ for a compound corresponds in each case to one row of table A

Table 466

Compounds of the formula I in which R^{a} is OC₂H₅, R^{b2} is OCH₃, R^{b3} is F and R^{b4} is Br and the combination of R¹, R³, R⁹ and R¹⁰ for a compound corresponds in each case to one row of table A

Table 467

Compounds of the formula I in which R^{a} is OC₂H₅, R^{b2} is OCH₃, R^{b3} is F and R^{b4} is CH₃ and the combination of R¹, R³, R⁹ and R¹⁰ for a compound corresponds in each case to one row of table A

Table 468

Compounds of the formula I in which R^{a} is OC₂H₅, R^{b2} is OCH₃, R^{b3} is F and R^{b4} is OCH₃ and the combination of R¹, R³, R⁹ and R¹⁰ for a compound corresponds in each case to one row of table A

Table 469

Compounds of the formula I in which R^{a} is morpholin-1-yl, R^{b2} is OCH₃ and R^{b3} is H and R^{b4} is H and the combination of R¹, R³, R⁹ and R¹⁰ for a compound corresponds in each case to one row of table A

Table 470

Compounds of the formula I in which R^{a} is morpholin-1-yl, R^{b2} is OCH₃ and R^{b3} is H and R^{b4} is F and the combination of R¹, R³, R⁹ and R¹⁰ for a compound corresponds in each case to one row of table A

Table 471

Compounds of the formula I in which R^{a} is morpholin-1-yl, R^{b2} is OCH₃ and R^{b3} is H and R^{b4} is Cl and the combination of R¹, R³, R⁹ and R¹⁰ for a compound corresponds in each case to one row of table A

Table 472

Compounds of the formula I in which R^{a} is morpholin-1-yl, R^{b2} is OCH₃ and R^{b3} is H and R^{b4} is Br and the combination of R¹, R³, R⁹ and R¹⁰ for a compound corresponds in each case to one row of table A

Table 473

Compounds of the formula I in which R^{a} is morpholin-1-yl, R^{b2} is OCH₃ and R^{b3} is H and R^{b4} is CH₃ and the combination of R¹, R³, R⁹ and R¹⁰ for a compound corresponds in each case to one row of table A

Table 474

Compounds of the formula I in which R^{a} is morpholin-1-yl, R^{b2} is OCH₃ and R^{b3} is H and R^{b4} is OCH₃ and the combination of R¹, R³, R⁹ and R¹⁰ for a compound corresponds in each case to one row of table A

Table 475

Compounds of the formula I in which R^{a} is morpholin-1-yl, R^{b2} is OCH₃, R^{b3} is F and R^{b4} is H and the combination of R¹, R³, R⁹ and R¹⁰ for a compound corresponds in each case to one row of table A

Table 476

Compounds of the formula I in which R^{a} is morpholin-1-yl, R^{b2} is OCH₃, R^{b3} is F and R^{b4} is F and the combination of R¹, R³, R⁹ and R¹⁰ for a compound corresponds in each case to one row of table A

Table 477

Compounds of the formula I in which R^{a} is morpholin-1-yl, R^{b2} is OCH₃, R^{b3} is F and R^{b4} is Cl and the combination of R¹, R³, R⁹ and R¹⁰ for a compound corresponds in each case to one row of table A

Table 478

Compounds of the formula I in which R^{a} is morpholin-1-yl, R^{b2} is OCH₃, R^{b3} is F and R^{b4} is Br and the combination of R¹, R³, R⁹ and R¹⁰ for a compound corresponds in each case to one row of table A

Table 479

Compounds of the formula I in which R^{a} is morpholin-1-yl, R^{b2} is OCH₃, R^{b3} is F and R^{b4} is CH₃ and the combination of R¹, R³, R⁹ and R¹⁰ for a compound corresponds in each case to one row of table A

Table 480

Compounds of the formula I in which R^{a} is morpholin-1-yl, R^{b2} is OCH₃, R^{b3} is F and R^{b4} is OCH₃ and the combination of R¹, R³, R⁹ and R¹⁰ for a compound corresponds in each case to one row of table A

TABLE A
Compounds of the formula I which correspond to the formulae
I.1A′, I.1B′, I.2A′ and I.2B′, respectively

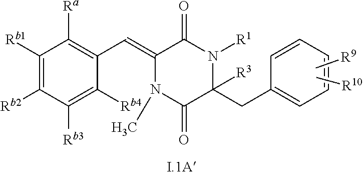

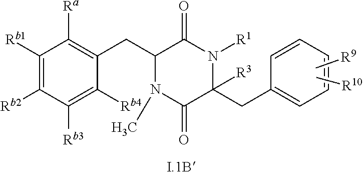

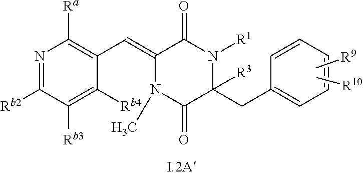

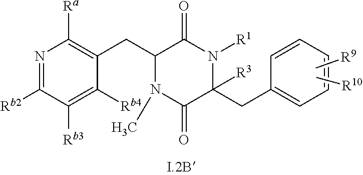

| No. | Formula | R¹ | R³ | R⁹ | R¹⁰ |
|---|---|---|---|---|---|
| A-1 | I.1A′ | CH₂CN | CH₃ | H | H |
| A-2 | I.1A′ | CH₂OCH₃ | CH₃ | H | H |
| A-3 | I.1A′ | CH₂-c-C₃H₅ | CH₃ | H | H |
| A-4 | I.1A′ | CH₂CH═CHCl | CH₃ | H | H |
| A-5 | I.1A′ | CH₂CN | CH₂CH₃ | H | H |
| A-6 | I.1A′ | CH₂OCH₃ | CH₂CH₃ | H | H |
| A-7 | I.1A′ | CH₂-c-C₃H₅ | CH₂CH₃ | H | H |
| A-8 | I.1A′ | CH₂CH═CHCl | CH₂CH₃ | H | H |
| A-9 | I.1A′ | CH₂CN | CH₂CH₂CH₃ | H | H |
| A-10 | I.1A′ | CH₂OCH₃ | CH₂CH₂CH₃ | H | H |
| A-11 | I.1A′ | CH₂-c-C₃H₅ | CH₂CH₂CH₃ | H | H |
| A-12 | I.1A′ | CH₂CH═CHCl | CH₂CH₂CH₃ | H | H |
| A-13 | I.1A′ | CH₂CN | OH | H | H |
| A-14 | I.1A′ | CH₂OCH₃ | OH | H | H |
| A-15 | I.1A′ | CH₂-c-C₃H₅ | OH | H | H |
| A-16 | I.1A′ | CH₂CH═CHCl | OH | H | H |
| A-17 | I.1A′ | CH₂CN | CH₂OH | H | H |
| A-18 | I.1A′ | CH₂OCH₃ | CH₂OH | H | H |
| A-19 | I.1A′ | CH₂-c-C₃H₅ | CH₂OH | H | H |
| A-20 | I.1A′ | CH₂CH═CHCl | CH₂OH | H | H |
| A-21 | I.1A′ | CH₂CN | NH₂ | H | H |
| A-22 | I.1A′ | CH₂OCH₃ | NH₂ | H | H |
| A-23 | I.1A′ | CH₂-c-C₃H₅ | NH₂ | H | H |
| A-24 | I.1A′ | CH₂CH═CHCl | NH₂ | H | H |
| A-25 | I.1A′ | CH₂CN | COOCH₃ | H | H |
| A-26 | I.1A′ | CH₂OCH₃ | COOCH₃ | H | H |
| A-27 | I.1A′ | CH₂-c-C₃H₅ | COOCH₃ | H | H |
| A-28 | I.1A′ | CH₂CH═CHCl | COOCH₃ | H | H |
| A-29 | I.1A′ | CH₂CN | CH₂CH═CH₂ | H | H |
| A-30 | I.1A′ | CH₂OCH₃ | CH₂CH═CH₂ | H | H |
| A-31 | I.1A′ | CH₂-c-C₃H₅ | CH₂CH═CH₂ | H | H |
| A-32 | I.1A′ | CH₂CH═CHCl | CH₂CH═CH₂ | H | H |
| A-33 | I.1A′ | CH₂CN | CH₃ | 2-F | H |
| A-34 | I.1A′ | CH₂OCH₃ | CH₃ | 2-F | H |
| A-35 | I.1A′ | CH₂-c-C₃H₅ | CH₃ | 2-F | H |
| A-36 | I.1A′ | CH₂CH═CHCl | CH₃ | 2-F | H |
| A-37 | I.1A′ | CH₂CN | CH₂CH₃ | 2-F | H |
| A-38 | I.1A′ | CH₂OCH₃ | CH₂CH₃ | 2-F | H |
| A-39 | I.1A′ | CH₂-c-C₃H₅ | CH₂CH₃ | 2-F | H |
| A-40 | I.1A′ | CH₂CH═CHCl | CH₂CH₃ | 2-F | H |
| A-41 | I.1A′ | CH₂CN | CH₂CH₂CH₃ | 2-F | H |
| A-42 | I.1A′ | CH₂OCH₃ | CH₂CH₂CH₃ | 2-F | H |
| A-43 | I.1A′ | CH₂-c-C₃H₅ | CH₂CH₂CH₃ | 2-F | H |
| A-44 | I.1A′ | CH₂CH═CHCl | CH₂CH₂CH₃ | 2-F | H |
| A-45 | I.1A′ | CH₂CN | OH | 2-F | H |
| A-46 | I.1A′ | CH₂OCH₃ | OH | 2-F | H |
| A-47 | I.1A′ | CH₂-c-C₃H₅ | OH | 2-F | H |
| A-48 | I.1A′ | CH₂CH═CHCl | OH | 2-F | H |
| A-49 | I.1A′ | CH₂CN | CH₂OH | 2-F | H |
| A-50 | I.1A′ | CH₂OCH₃ | CH₂OH | 2-F | H |
| A-51 | I.1A′ | CH₂-c-C₃H₅ | CH₂OH | 2-F | H |
| A-52 | I.1A′ | CH₂CH═CHCl | CH₂OH | 2-F | H |
| A-53 | I.1A′ | CH₂CN | NH₂ | 2-F | H |
| A-54 | I.1A′ | CH₂OCH₃ | NH₂ | 2-F | H |
| A-55 | I.1A′ | CH₂-c-C₃H₅ | NH₂ | 2-F | H |
| A-56 | I.1A′ | CH₂CH═CHCl | NH₂ | 2-F | H |
| A-57 | I.1A′ | CH₂CN | COOCH₃ | 2-F | H |
| A-58 | I.1A′ | CH₂OCH₃ | COOCH₃ | 2-F | H |
| A-59 | I.1A′ | CH₂-c-C₃H₅ | COOCH₃ | 2-F | H |
| A-60 | I.1A′ | CH₂CH═CHCl | COOCH₃ | 2-F | H |
| A-61 | I.1A′ | CH₂CN | CH₂CH═CH₂ | 2-F | H |
| A-62 | I.1A′ | CH₂OCH₃ | CH₂CH═CH₂ | 2-F | H |
| A-63 | I.1A′ | CH₂-c-C₃H₅ | CH₂CH═CH₂ | 2-F | H |
| A-64 | I.1A′ | CH₂CH═CHCl | CH₂CH═CH₂ | 2-F | H |
| A-65 | I.1A′ | CH₂CN | CH₃ | 3-F | H |
| A-66 | I.1A′ | CH₂OCH₃ | CH₃ | 3-F | H |
| A-67 | I.1A′ | CH₂-c-C₃H₅ | CH₃ | 3-F | H |
| A-68 | I.1A′ | CH₂CH═CHCl | CH₃ | 3-F | H |
| A-69 | I.1A′ | CH₂CN | CH₂CH₃ | 3-F | H |
| A-70 | I.1A′ | CH₂OCH₃ | CH₂CH₃ | 3-F | H |
| A-71 | I.1A′ | CH₂-c-C₃H₅ | CH₂CH₃ | 3-F | H |
| A-72 | I.1A′ | CH₂CH═CHCl | CH₂CH₃ | 3-F | H |
| A-73 | I.1A′ | CH₂CN | CH₂CH₂CH₃ | 3-F | H |
| A-74 | I.1A′ | CH₂OCH₃ | CH₂CH₂CH₃ | 3-F | H |
| A-75 | I.1A′ | CH₂-c-C₃H₅ | CH₂CH₂CH₃ | 3-F | H |
| A-76 | I.1A′ | CH₂CH═CHCl | CH₂CH₂CH₃ | 3-F | H |
| A-77 | I.1A′ | CH₂CN | OH | 3-F | H |
| A-78 | I.1A′ | CH₂OCH₃ | OH | 3-F | H |
| A-79 | I.1A′ | CH₂-c-C₃H₅ | OH | 3-F | H |
| A-80 | I.1A′ | CH₂CH═CHCl | OH | 3-F | H |
| A-81 | I.1A′ | CH₂CN | CH₂OH | 3-F | H |
| A-82 | I.1A′ | CH₂OCH₃ | CH₂OH | 3-F | H |
| A-83 | I.1A′ | CH₂-c-C₃H₅ | CH₂OH | 3-F | H |
| A-84 | I.1A′ | CH₂CH═CHCl | CH₂OH | 3-F | H |
| A-85 | I.1A′ | CH₂CN | NH₂ | 3-F | H |
| A-86 | I.1A′ | CH₂OCH₃ | NH₂ | 3-F | H |
| A-87 | I.1A′ | CH₂-c-C₃H₅ | NH₂ | 3-F | H |
| A-88 | I.1A′ | CH₂CH═CHCl | NH₂ | 3-F | H |
| A-89 | I.1A′ | CH₂CN | COOCH₃ | 3-F | H |
| A-90 | I.1A′ | CH₂OCH₃ | COOCH₃ | 3-F | H |
| A-91 | I.1A′ | CH₂-c-C₃H₅ | COOCH₃ | 3-F | H |
| A-92 | I.1A′ | CH₂CH═CHCl | COOCH₃ | 3-F | H |
| A-93 | I.1A′ | CH₂CN | CH₂CH═CH₂ | 3-F | H |
| A-94 | I.1A′ | CH₂OCH₃ | CH₂CH═CH₂ | 3-F | H |
| A-95 | I.1A′ | CH₂-c-C₃H₅ | CH₂CH═CH₂ | 3-F | H |
| A-96 | I.1A′ | CH₂CH═CHCl | CH₂CH═CH₂ | 3-F | H |
| A-97 | I.1A′ | CH₂CN | CH₃ | 4-F | H |
| A-98 | I.1A′ | CH₂OCH₃ | CH₃ | 4-F | H |
| A-99 | I.1A′ | CH₂-c-C₃H₅ | CH₃ | 4-F | H |
| A-100 | I.1A′ | CH₂CH═CHCl | CH₃ | 4-F | H |
| A-101 | I.1A′ | CH₂CN | CH₂CH₃ | 4-F | H |
| A-102 | I.1A′ | CH₂OCH₃ | CH₂CH₃ | 4-F | H |
| A-103 | I.1A′ | CH₂-c-C₃H₅ | CH₂CH₃ | 4-F | H |
| A-104 | I.1A′ | CH₂CH═CHCl | CH₂CH₃ | 4-F | H |
| A-105 | I.1A′ | CH₂CN | CH₂CH₂CH₃ | 4-F | H |
| A-106 | I.1A′ | CH₂OCH₃ | CH₂CH₂CH₃ | 4-F | H |
| A-107 | I.1A′ | CH₂-c-C₃H₅ | CH₂CH₂CH₃ | 4-F | H |
| A-108 | I.1A′ | CH₂CH═CHCl | CH₂CH₂CH₃ | 4-F | H |
| A-109 | I.1A′ | CH₂CN | OH | 4-F | H |
| A-110 | I.1A′ | CH₂OCH₃ | OH | 4-F | H |
| A-111 | I.1A′ | CH₂-c-C₃H₅ | OH | 4-F | H |
| A-112 | I.1A′ | CH₂CH═CHCl | OH | 4-F | H |
| A-113 | I.1A′ | CH₂CN | CH₂OH | 4-F | H |
| A-114 | I.1A′ | CH₂OCH₃ | CH₂OH | 4-F | H |
| A-115 | I.1A′ | CH₂-c-C₃H₅ | CH₂OH | 4-F | H |
| A-116 | I.1A′ | CH₂CH═CHCl | CH₂OH | 4-F | H |
| A-117 | I.1A′ | CH₂CN | NH₂ | 4-F | H |
| A-118 | I.1A′ | CH₂OCH₃ | NH₂ | 4-F | H |
| A-119 | I.1A′ | CH₂-c-C₃H₅ | NH₂ | 4-F | H |
| A-120 | I.1A′ | CH₂CH═CHCl | NH₂ | 4-F | H |
| A-121 | I.1A′ | CH₂CN | COOCH₃ | 4-F | H |
| A-122 | I.1A′ | CH₂OCH₃ | COOCH₃ | 4-F | H |
| A-123 | I.1A′ | CH₂-c-C₃H₅ | COOCH₃ | 4-F | H |
| A-124 | I.1A′ | CH₂CH═CHCl | COOCH₃ | 4-F | H |
| A-125 | I.1A′ | CH₂CN | CH₂CH═CH₂ | 4-F | H |
| A-126 | I.1A′ | CH₂OCH₃ | CH₂CH═CH₂ | 4-F | H |
| A-127 | I.1A′ | CH₂-c-C₃H₅ | CH₂CH═CH₂ | 4-F | H |
| A-128 | I.1A′ | CH₂CH═CHCl | CH₂CH═CH₂ | 4-F | H |
| A-129 | I.1A′ | CH₂CN | CH₃ | 3-F | 2-F |
| A-130 | I.1A′ | CH₂OCH₃ | CH₃ | 3-F | 2-F |
| A-131 | I.1A′ | CH₂-c-C₃H₅ | CH₃ | 3-F | 2-F |
| A-132 | I.1A′ | CH₂CH═CHCl | CH₃ | 3-F | 2-F |
| A-133 | I.1A′ | CH₂CN | CH₂CH₃ | 3-F | 2-F |
| A-134 | I.1A′ | CH₂OCH₃ | CH₂CH₃ | 3-F | 2-F |
| A-135 | I.1A′ | CH₂-c-C₃H₅ | CH₂CH₃ | 3-F | 2-F |
| A-136 | I.1A′ | CH₂CH═CHCl | CH₂CH₃ | 3-F | 2-F |
| A-137 | I.1A′ | CH₂CN | CH₂CH₂CH₃ | 3-F | 2-F |
| A-138 | I.1A′ | CH₂OCH₃ | CH₂CH₂CH₃ | 3-F | 2-F |
| A-139 | I.1A′ | CH₂-c-C₃H₅ | CH₂CH₂CH₃ | 3-F | 2-F |
| A-140 | I.1A′ | CH₂CH═CHCl | CH₂CH₂CH₃ | 3-F | 2-F |
| A-141 | I.1A′ | CH₂CN | OH | 3-F | 2-F |
| A-142 | I.1A′ | CH₂OCH₃ | OH | 3-F | 2-F |
| A-143 | I.1A′ | CH₂-c-C₃H₅ | OH | 3-F | 2-F |
| A-144 | I.1A′ | CH₂CH═CHCl | OH | 3-F | 2-F |
| A-145 | I.1A′ | CH₂CN | CH₂OH | 3-F | 2-F |
| A-146 | I.1A′ | CH₂OCH₃ | CH₂OH | 3-F | 2-F |
| A-147 | I.1A′ | CH₂-c-C₃H₅ | CH₂OH | 3-F | 2-F |
| A-148 | I.1A′ | CH₂CH═CHCl | CH₂OH | 3-F | 2-F |
| A-149 | I.1A′ | CH₂CN | NH₂ | 3-F | 2-F |
| A-150 | I.1A′ | CH₂OCH₃ | NH₂ | 3-F | 2-F |
| A-151 | I.1A′ | CH₂-c-C₃H₅ | NH₂ | 3-F | 2-F |
| A-152 | I.1A′ | CH₂CH═CHCl | NH₂ | 3-F | 2-F |
| A-153 | I.1A′ | CH₂CN | COOCH₃ | 3-F | 2-F |
| A-154 | I.1A′ | CH₂OCH₃ | COOCH₃ | 3-F | 2-F |
| A-155 | I.1A′ | CH₂-c-C₃H₅ | COOCH₃ | 3-F | 2-F |
| A-156 | I.1A′ | CH₂CH═CHCl | COOCH₃ | 3-F | 2-F |
| A-157 | I.1A′ | CH₂CN | CH₂CH═CH₂ | 3-F | 2-F |
| A-158 | I.1A′ | CH₂OCH₃ | CH₂CH═CH₂ | 3-F | 2-F |
| A-159 | I.1A′ | CH₂-c-C₃H₅ | CH₂CH═CH₂ | 3-F | 2-F |
| A-160 | I.1A′ | CH₂CH═CHCl | CH₂CH═CH₂ | 3-F | 2-F |
| A-161 | I.1A′ | CH₂CN | CH₃ | 3-F | 4-F |
| A-162 | I.1A′ | CH₂OCH₃ | CH₃ | 3-F | 4-F |
| A-163 | I.1A′ | CH₂-c-C₃H₅ | CH₃ | 3-F | 4-F |
| A-164 | I.1A′ | CH₂CH═CHCl | CH₃ | 3-F | 4-F |
| A-165 | I.1A′ | CH₂CN | CH₂CH₃ | 3-F | 4-F |
| A-166 | I.1A′ | CH₂OCH₃ | CH₂CH₃ | 3-F | 4-F |
| A-167 | I.1A′ | CH₂-c-C₃H₅ | CH₂CH₃ | 3-F | 4-F |
| A-168 | I.1A′ | CH₂CH═CHCl | CH₂CH₃ | 3-F | 4-F |
| A-169 | I.1A′ | CH₂CN | CH₂CH₂CH₃ | 3-F | 4-F |
| A-170 | I.1A′ | CH₂OCH₃ | CH₂CH₂CH₃ | 3-F | 4-F |
| A-171 | I.1A′ | CH₂-c-C₃H₅ | CH₂CH₂CH₃ | 3-F | 4-F |
| A-172 | I.1A′ | CH₂CH═CHCl | CH₂CH₂CH₃ | 3-F | 4-F |
| A-173 | I.1A′ | CH₂CN | OH | 3-F | 4-F |
| A-174 | I.1A′ | CH₂OCH₃ | OH | 3-F | 4-F |
| A-175 | I.1A′ | CH₂-c-C₃H₅ | OH | 3-F | 4-F |
| A-176 | I.1A′ | CH₂CH═CHCl | OH | 3-F | 4-F |
| A-177 | I.1A′ | CH₂CN | CH₂OH | 3-F | 4-F |
| A-178 | I.1A′ | CH₂OCH₃ | CH₂OH | 3-F | 4-F |
| A-179 | I.1A′ | CH₂-c-C₃H₅ | CH₂OH | 3-F | 4-F |
| A-180 | I.1A′ | CH₂CH═CHCl | CH₂OH | 3-F | 4-F |
| A-181 | I.1A′ | CH₂CN | NH₂ | 3-F | 4-F |
| A-182 | I.1A′ | CH₂OCH₃ | NH₂ | 3-F | 4-F |
| A-183 | I.1A′ | CH₂-c-C₃H₅ | NH₂ | 3-F | 4-F |
| A-184 | I.1A′ | CH₂CH═CHCl | NH₂ | 3-F | 4-F |
| A-185 | I.1A′ | CH₂CN | COOCH₃ | 3-F | 4-F |
| A-186 | I.1A′ | CH₂OCH₃ | COOCH₃ | 3-F | 4-F |
| A-187 | I.1A′ | CH₂-c-C₃H₅ | COOCH₃ | 3-F | 4-F |
| A-188 | I.1A′ | CH₂CH═CHCl | COOCH₃ | 3-F | 4-F |
| A-189 | I.1A′ | CH₂CN | CH₂CH═CH₂ | 3-F | 4-F |
| A-190 | I.1A′ | CH₂OCH₃ | CH₂CH═CH₂ | 3-F | 4-F |
| A-191 | I.1A′ | CH₂-c-C₃H₅ | CH₂CH═CH₂ | 3-F | 4-F |
| A-192 | I.1A′ | CH₂CH═CHCl | CH₂CH═CH₂ | 3-F | 4-F |
| A-193 | I.1B′ | CH₂CN | CH₃ | H | H |
| A-194 | I.1B′ | CH₂OCH₃ | CH₃ | H | H |
| A-195 | I.1B′ | CH₂-c-C₃H₅ | CH₃ | H | H |
| A-196 | I.1B′ | CH₂CH═CHCl | CH₃ | H | H |
| A-197 | I.1B′ | CH₂CN | CH₂CH₃ | H | H |
| A-198 | I.1B′ | CH₂OCH₃ | CH₂CH₃ | H | H |
| A-199 | I.1B′ | CH₂-c-C₃H₅ | CH₂CH₃ | H | H |
| A-200 | I.1B′ | CH₂CH═CHCl | CH₂CH₃ | H | H |
| A-201 | I.1B′ | CH₂CN | CH₂CH₂CH₃ | H | H |
| A-202 | I.1B′ | CH₂OCH₃ | CH₂CH₂CH₃ | H | H |
| A-203 | I.1B′ | CH₂-c-C₃H₅ | CH₂CH₂CH₃ | H | H |
| A-204 | I.1B′ | CH₂CH═CHCl | CH₂CH₂CH₃ | H | H |
| A-205 | I.1B′ | CH₂CN | OH | H | H |
| A-206 | I.1B′ | CH₂OCH₃ | OH | H | H |
| A-207 | I.1B′ | CH₂-c-C₃H₅ | OH | H | H |
| A-208 | I.1B′ | CH₂CH═CHCl | OH | H | H |
| A-209 | I.1B′ | CH₂CN | CH₂OH | H | H |
| A-210 | I.1B′ | CH₂OCH₃ | CH₂OH | H | H |
| A-211 | I.1B′ | CH₂-c-C₃H₅ | CH₂OH | H | H |
| A-212 | I.1B′ | CH₂CH═CHCl | CH₂OH | H | H |
| A-213 | I.1B′ | CH₂CN | NH₂ | H | H |
| A-214 | I.1B′ | CH₂OCH₃ | NH₂ | H | H |
| A-215 | I.1B′ | CH₂-c-C₃H₅ | NH₂ | H | H |
| A-216 | I.1B′ | CH₂CH═CHCl | NH₂ | H | H |
| A-217 | I.1B′ | CH₂CN | COOCH₃ | H | H |
| A-218 | I.1B′ | CH₂OCH₃ | COOCH₃ | H | H |
| A-219 | I.1B′ | CH₂-c-C₃H₅ | COOCH₃ | H | H |
| A-220 | I.1B′ | CH₂CH═CHCl | COOCH₃ | H | H |
| A-221 | I.1B′ | CH₂CN | CH₂CH═CH₂ | H | H |
| A-222 | I.1B′ | CH₂OCH₃ | CH₂CH═CH₂ | H | H |
| A-223 | I.1B′ | CH₂-c-C₃H₅ | CH₂CH═CH₂ | H | H |
| A-224 | I.1B′ | CH₂CH═CHCl | CH₂CH═CH₂ | H | H |
| A-225 | I.1B′ | CH₂CN | CH₃ | 2-F | H |
| A-226 | I.1B′ | CH₂OCH₃ | CH₃ | 2-F | H |
| A-227 | I.1B′ | CH₂-c-C₃H₅ | CH₃ | 2-F | H |
| A-228 | I.1B′ | CH₂CH═CHCl | CH₃ | 2-F | H |
| A-229 | I.1B′ | CH₂CN | CH₂CH₃ | 2-F | H |
| A-230 | I.1B′ | CH₂OCH₃ | CH₂CH₃ | 2-F | H |
| A-231 | I.1B′ | CH₂-c-C₃H₅ | CH₂CH₃ | 2-F | H |
| A-232 | I.1B′ | CH₂CH═CHCl | CH₂CH₃ | 2-F | H |
| A-233 | I.1B′ | CH₂CN | CH₂CH₂CH₃ | 2-F | H |
| A-234 | I.1B′ | CH₂OCH₃ | CH₂CH₂CH₃ | 2-F | H |
| A-235 | I.1B′ | CH₂-c-C₃H₅ | CH₂CH₂CH₃ | 2-F | H |
| A-236 | I.1B′ | CH₂CH═CHCl | CH₂CH₂CH₃ | 2-F | H |
| A-237 | I.1B′ | CH₂CN | OH | 2-F | H |
| A-238 | I.1B′ | CH₂OCH₃ | OH | 2-F | H |
| A-239 | I.1B′ | CH₂-c-C₃H₅ | OH | 2-F | H |
| A-240 | I.1B′ | CH₂CH═CHCl | OH | 2-F | H |
| A-241 | I.1B′ | CH₂CN | CH₂OH | 2-F | H |
| A-242 | I.1B′ | CH₂OCH₃ | CH₂OH | 2-F | H |
| A-243 | I.1B′ | CH₂-c-C₃H₅ | CH₂OH | 2-F | H |
| A-244 | I.1B′ | CH₂CH═CHCl | CH₂OH | 2-F | H |
| A-245 | I.1B′ | CH₂CN | NH₂ | 2-F | H |
| A-246 | I.1B′ | CH₂OCH₃ | NH₂ | 2-F | H |
| A-247 | I.1B′ | CH₂-c-C₃H₅ | NH₂ | 2-F | H |
| A-248 | I.1B′ | CH₂CH═CHCl | NH₂ | 2-F | H |
| A-249 | I.1B′ | CH₂CN | COOCH₃ | 2-F | H |
| A-250 | I.1B′ | CH₂OCH₃ | COOCH₃ | 2-F | H |
| A-251 | I.1B′ | CH₂-c-C₃H₅ | COOCH₃ | 2-F | H |
| A-252 | I.1B′ | CH₂CH═CHCl | COOCH₃ | 2-F | H |
| A-253 | I.1B′ | CH₂CN | CH₂CH═CH₂ | 2-F | H |
| A-254 | I.1B′ | CH₂OCH₃ | CH₂CH═CH₂ | 2-F | H |
| A-255 | I.1B′ | CH₂-c-C₃H₅ | CH₂CH═CH₂ | 2-F | H |
| A-256 | I.1B′ | CH₂CH═CHCl | CH₂CH═CH₂ | 2-F | H |
| A-257 | I.1B′ | CH₂CN | CH₃ | 3-F | H |
| A-258 | I.1B′ | CH₂OCH₃ | CH₃ | 3-F | H |
| A-259 | I.1B′ | CH₂-c-C₃H₅ | CH₃ | 3-F | H |
| A-260 | I.1B′ | CH₂CH═CHCl | CH₃ | 3-F | H |
| A-261 | I.1B′ | CH₂CN | CH₂CH₃ | 3-F | H |
| A-262 | I.1B′ | CH₂OCH₃ | CH₂CH₃ | 3-F | H |
| A-263 | I.1B′ | CH₂-c-C₃H₅ | CH₂CH₃ | 3-F | H |
| A-264 | I.1B′ | CH₂CH═CHCl | CH₂CH₃ | 3-F | H |
| A-265 | I.1B′ | CH₂CN | CH₂CH₂CH₃ | 3-F | H |
| A-266 | I.1B′ | CH₂OCH₃ | CH₂CH₂CH₃ | 3-F | H |
| A-267 | I.1B′ | CH₂-c-C₃H₅ | CH₂CH₂CH₃ | 3-F | H |
| A-268 | I.1B′ | CH₂CH═CHCl | CH₂CH₂CH₃ | 3-F | H |
| A-269 | I.1B′ | CH₂CN | OH | 3-F | H |
| A-270 | I.1B′ | CH₂OCH₃ | OH | 3-F | H |
| A-271 | I.1B′ | CH₂-c-C₃H₅ | OH | 3-F | H |
| A-272 | I.1B′ | CH₂CH═CHCl | OH | 3-F | H |
| A-273 | I.1B′ | CH₂CN | CH₂OH | 3-F | H |
| A-274 | I.1B′ | CH₂OCH₃ | CH₂OH | 3-F | H |
| A-275 | I.1B′ | CH₂-c-C₃H₅ | CH₂OH | 3-F | H |
| A-276 | I.1B′ | CH₂CH═CHCl | CH₂OH | 3-F | H |
| A-277 | I.1B′ | CH₂CN | NH₂ | 3-F | H |
| A-278 | I.1B′ | CH₂OCH₃ | NH₂ | 3-F | H |
| A-279 | I.1B′ | CH₂-c-C₃H₅ | NH₂ | 3-F | H |
| A-280 | I.1B′ | CH₂CH═CHCl | NH₂ | 3-F | H |
| A-281 | I.1B′ | CH₂CN | COOCH₃ | 3-F | H |
| A-282 | I.1B′ | CH₂OCH₃ | COOCH₃ | 3-F | H |
| A-283 | I.1B′ | CH₂-c-C₃H₅ | COOCH₃ | 3-F | H |
| A-284 | I.1B′ | CH₂CH═CHCl | COOCH₃ | 3-F | H |
| A-285 | I.1B′ | CH₂CN | CH₂CH═CH₂ | 3-F | H |
| A-286 | I.1B′ | CH₂OCH₃ | CH₂CH═CH₂ | 3-F | H |
| A-287 | I.1B′ | CH₂-c-C₃H₅ | CH₂CH═CH₂ | 3-F | H |
| A-288 | I.1B′ | CH₂CH═CHCl | CH₂CH═CH₂ | 3-F | H |
| A-289 | I.1B′ | CH₂CN | CH₃ | 4-F | H |
| A-290 | I.1B′ | CH₂OCH₃ | CH₃ | 4-F | H |
| A-291 | I.1B′ | CH₂-c-C₃H₅ | CH₃ | 4-F | H |
| A-292 | I.1B′ | CH₂CH═CHCl | CH₃ | 4-F | H |
| A-293 | I.1B′ | CH₂CN | CH₂CH₃ | 4-F | H |
| A-294 | I.1B′ | CH₂OCH₃ | CH₂CH₃ | 4-F | H |
| A-295 | I.1B′ | CH₂-c-C₃H₅ | CH₂CH₃ | 4-F | H |
| A-296 | I.1B′ | CH₂CH═CHCl | CH₂CH₃ | 4-F | H |
| A-297 | I.1B′ | CH₂CN | CH₂CH₂CH₃ | 4-F | H |
| A-298 | I.1B′ | CH₂OCH₃ | CH₂CH₂CH₃ | 4-F | H |
| A-299 | I.1B′ | CH₂-c-C₃H₅ | CH₂CH₂CH₃ | 4-F | H |
| A-300 | I.1B′ | CH₂CH═CHCl | CH₂CH₂CH₃ | 4-F | H |
| A-301 | I.1B′ | CH₂CN | OH | 4-F | H |
| A-302 | I.1B′ | CH₂OCH₃ | OH | 4-F | H |
| A-303 | I.1B′ | CH₂-c-C₃H₅ | OH | 4-F | H |
| A-304 | I.1B′ | CH₂CH═CHCl | OH | 4-F | H |
| A-305 | I.1B′ | CH₂CN | CH₂OH | 4-F | H |
| A-306 | I.1B′ | CH₂OCH₃ | CH₂OH | 4-F | H |
| A-307 | I.1B′ | CH₂-c-C₃H₅ | CH₂OH | 4-F | H |
| A-308 | I.1B′ | CH₂CH═CHCl | CH₂OH | 4-F | H |
| A-309 | I.1B′ | CH₂CN | NH₂ | 4-F | H |
| A-310 | I.1B′ | CH₂OCH₃ | NH₂ | 4-F | H |
| A-311 | I.1B′ | CH₂-c-C₃H₅ | NH₂ | 4-F | H |
| A-312 | I.1B′ | CH₂CH═CHCl | NH₂ | 4-F | H |
| A-313 | I.1B′ | CH₂CN | COOCH₃ | 4-F | H |
| A-314 | I.1B′ | CH₂OCH₃ | COOCH₃ | 4-F | H |
| A-315 | I.1B′ | CH₂-c-C₃H₅ | COOCH₃ | 4-F | H |
| A-316 | I.1B′ | CH₂CH═CHCl | COOCH₃ | 4-F | H |
| A-317 | I.1B′ | CH₂CN | CH₂CH═CH₂ | 4-F | H |
| A-318 | I.1B′ | CH₂OCH₃ | CH₂CH═CH₂ | 4-F | H |
| A-319 | I.1B′ | CH₂-c-C₃H₅ | CH₂CH═CH₂ | 4-F | H |
| A-320 | I.1B′ | CH₂CH═CHCl | CH₂CH═CH₂ | 4-F | H |
| A-321 | I.1B′ | CH₂CN | CH₃ | 3-F | 2-F |
| A-322 | I.1B′ | CH₂OCH₃ | CH₃ | 3-F | 2-F |
| A-323 | I.1B′ | CH₂-c-C₃H₅ | CH₃ | 3-F | 2-F |
| A-324 | I.1B′ | CH₂CH═CHCl | CH₃ | 3-F | 2-F |
| A-325 | I.1B′ | CH₂CN | CH₂CH₃ | 3-F | 2-F |
| A-326 | I.1B′ | CH₂OCH₃ | CH₂CH₃ | 3-F | 2-F |
| A-327 | I.1B′ | CH₂-c-C₃H₅ | CH₂CH₃ | 3-F | 2-F |
| A-328 | I.1B′ | CH₂CH═CHCl | CH₂CH₃ | 3-F | 2-F |
| A-329 | I.1B′ | CH₂CN | CH₂CH₂CH₃ | 3-F | 2-F |
| A-330 | I.1B′ | CH₂OCH₃ | CH₂CH₂CH₃ | 3-F | 2-F |
| A-331 | I.1B′ | CH₂-c-C₃H₅ | CH₂CH₂CH₃ | 3-F | 2-F |
| A-332 | I.1B′ | CH₂CH═CHCl | CH₂CH₂CH₃ | 3-F | 2-F |
| A-333 | I.1B′ | CH₂CN | OH | 3-F | 2-F |
| A-334 | I.1B′ | CH₂OCH₃ | OH | 3-F | 2-F |
| A-335 | I.1B′ | CH₂-c-C₃H₅ | OH | 3-F | 2-F |
| A-336 | I.1B′ | CH₂CH═CHCl | OH | 3-F | 2-F |
| A-337 | I.1B′ | CH₂CN | CH₂OH | 3-F | 2-F |
| A-338 | I.1B′ | CH₂OCH₃ | CH₂OH | 3-F | 2-F |
| A-339 | I.1B′ | CH₂-c-C₃H₅ | CH₂OH | 3-F | 2-F |
| A-340 | I.1B′ | CH₂CH═CHCl | CH₂OH | 3-F | 2-F |
| A-341 | I.1B′ | CH₂CN | NH₂ | 3-F | 2-F |
| A-342 | I.1B′ | CH₂OCH₃ | NH₂ | 3-F | 2-F |
| A-343 | I.1B′ | CH₂-c-C₃H₅ | NH₂ | 3-F | 2-F |
| A-344 | I.1B′ | CH₂CH═CHCl | NH₂ | 3-F | 2-F |
| A-345 | I.1B′ | CH₂CN | COOCH₃ | 3-F | 2-F |
| A-346 | I.1B′ | CH₂OCH₃ | COOCH₃ | 3-F | 2-F |
| A-347 | I.1B′ | CH₂-c-C₃H₅ | COOCH₃ | 3-F | 2-F |
| A-348 | I.1B′ | CH₂CH═CHCl | COOCH₃ | 3-F | 2-F |
| A-349 | I.1B′ | CH₂CN | CH₂CH═CH₂ | 3-F | 2-F |
| A-350 | I.1B′ | CH₂OCH₃ | CH₂CH═CH₂ | 3-F | 2-F |
| A-351 | I.1B′ | CH₂-c-C₃H₅ | CH₂CH═CH₂ | 3-F | 2-F |
| A-352 | I.1B′ | CH₂CH═CHCl | CH₂CH═CH₂ | 3-F | 2-F |
| A-353 | I.1B′ | CH₂CN | CH₃ | 3-F | 4-F |
| A-354 | I.1B′ | CH₂OCH₃ | CH₃ | 3-F | 4-F |
| A-355 | I.1B′ | CH₂-c-C₃H₅ | CH₃ | 3-F | 4-F |
| A-356 | I.1B′ | CH₂CH═CHCl | CH₃ | 3-F | 4-F |
| A-357 | I.1B′ | CH₂CN | CH₂CH₃ | 3-F | 4-F |
| A-358 | I.1B′ | CH₂OCH₃ | CH₂CH₃ | 3-F | 4-F |
| A-359 | I.1B′ | CH₂-c-C₃H₅ | CH₂CH₃ | 3-F | 4-F |
| A-360 | I.1B′ | CH₂CH═CHCl | CH₂CH₃ | 3-F | 4-F |
| A-361 | I.1B′ | CH₂CN | CH₂CH₂CH₃ | 3-F | 4-F |
| A-362 | I.1B′ | CH₂OCH₃ | CH₂CH₂CH₃ | 3-F | 4-F |
| A-363 | I.1B′ | CH₂-c-C₃H₅ | CH₂CH₂CH₃ | 3-F | 4-F |
| A-364 | I.1B′ | CH₂CH═CHCl | CH₂CH₂CH₃ | 3-F | 4-F |
| A-365 | I.1B′ | CH₂CN | OH | 3-F | 4-F |
| A-366 | I.1B′ | CH₂OCH₃ | OH | 3-F | 4-F |
| A-367 | I.1B′ | CH₂-c-C₃H₅ | OH | 3-F | 4-F |
| A-368 | I.1B′ | CH₂CH═CHCl | OH | 3-F | 4-F |
| A-369 | I.1B′ | CH₂CN | CH₂OH | 3-F | 4-F |
| A-370 | I.1B′ | CH₂OCH₃ | CH₂OH | 3-F | 4-F |
| A-371 | I.1B′ | CH₂-c-C₃H₅ | CH₂OH | 3-F | 4-F |
| A-372 | I.1B′ | CH₂CH═CHCl | CH₂OH | 3-F | 4-F |
| A-373 | I.1B′ | CH₂CN | NH₂ | 3-F | 4-F |
| A-374 | I.1B′ | CH₂OCH₃ | NH₂ | 3-F | 4-F |
| A-375 | I.1B′ | CH₂-c-C₃H₅ | NH₂ | 3-F | 4-F |
| A-376 | I.1B′ | CH₂CH═CHCl | NH₂ | 3-F | 4-F |
| A-377 | I.1B′ | CH₂CN | COOCH₃ | 3-F | 4-F |
| A-378 | I.1B′ | CH₂OCH₃ | COOCH₃ | 3-F | 4-F |
| A-379 | I.1B′ | CH₂-c-C₃H₅ | COOCH₃ | 3-F | 4-F |
| A-380 | I.1B′ | CH₂CH═CHCl | COOCH₃ | 3-F | 4-F |
| A-381 | I.1B′ | CH₂CN | CH₂CH═CH₂ | 3-F | 4-F |
| A-382 | I.1B′ | CH₂OCH₃ | CH₂CH═CH₂ | 3-F | 4-F |
| A-383 | I.1B′ | CH₂-c-C₃H₅ | CH₂CH═CH₂ | 3-F | 4-F |
| A-384 | I.1B′ | CH₂CH═CHCl | CH₂CH═CH₂ | 3-F | 4-F |
| A-385 | I.2A′ | CH₂CN | CH₃ | H | H |
| A-386 | I.2A′ | CH₂OCH₃ | CH₃ | H | H |
| A-387 | I.2A′ | CH₂-c-C₃H₅ | CH₃ | H | H |
| A-388 | I.2A′ | CH₂CH═CHCl | CH₃ | H | H |
| A-389 | I.2A′ | CH₂CN | CH₂CH₃ | H | H |
| A-390 | I.2A′ | CH₂OCH₃ | CH₂CH₃ | H | H |
| A-391 | I.2A′ | CH₂-c-C₃H₅ | CH₂CH₃ | H | H |
| A-392 | I.2A′ | CH₂CH═CHCl | CH₂CH₃ | H | H |
| A-393 | I.2A′ | CH₂CN | CH₂CH₂CH₃ | H | H |
| A-394 | I.2A′ | CH₂OCH₃ | CH₂CH₂CH₃ | H | H |
| A-395 | I.2A′ | CH₂-c-C₃H₅ | CH₂CH₂CH₃ | H | H |
| A-396 | I.2A′ | CH₂CH═CHCl | CH₂CH₂CH₃ | H | H |
| A-397 | I.2A′ | CH₂CN | OH | H | H |
| A-398 | I.2A′ | CH₂OCH₃ | OH | H | H |
| A-399 | I.2A′ | CH₂-c-C₃H₅ | OH | H | H |
| A-400 | I.2A′ | CH₂CH═CHCl | OH | H | H |
| A-401 | I.2A′ | CH₂CN | CH₂OH | H | H |
| A-402 | I.2A′ | CH₂OCH₃ | CH₂OH | H | H |
| A-403 | I.2A′ | CH₂-c-C₃H₅ | CH₂OH | H | H |
| A-404 | I.2A′ | CH₂CH═CHCl | CH₂OH | H | H |
| A-405 | I.2A′ | CH₂CN | NH₂ | H | H |
| A-406 | I.2A′ | CH₂OCH₃ | NH₂ | H | H |
| A-407 | I.2A′ | CH₂-c-C₃H₅ | NH₂ | H | H |
| A-408 | I.2A′ | CH₂CH═CHCl | NH₂ | H | H |
| A-409 | I.2A′ | CH₂CN | COOCH₃ | H | H |
| A-410 | I.2A′ | CH₂OCH₃ | COOCH₃ | H | H |
| A-411 | I.2A′ | CH₂-c-C₃H₅ | COOCH₃ | H | H |
| A-412 | I.2A′ | CH₂CH═CHCl | COOCH₃ | H | H |
| A-413 | I.2A′ | CH₂CN | CH₂CH═CH₂ | H | H |
| A-414 | I.2A′ | CH₂OCH₃ | CH₂CH═CH₂ | H | H |
| A-415 | I.2A′ | CH₂-c-C₃H₅ | CH₂CH═CH₂ | H | H |
| A-416 | I.2A′ | CH₂CH═CHCl | CH₂CH═CH₂ | H | H |
| A-417 | I.2A′ | CH₂CN | CH₃ | 2-F | H |
| A-418 | I.2A′ | CH₂OCH₃ | CH₃ | 2-F | H |
| A-419 | I.2A′ | CH₂-c-C₃H₅ | CH₃ | 2-F | H |
| A-420 | I.2A′ | CH₂CH═CHCl | CH₃ | 2-F | H |
| A-421 | I.2A′ | CH₂CN | CH₂CH₃ | 2-F | H |
| A-422 | I.2A′ | CH₂OCH₃ | CH₂CH₃ | 2-F | H |
| A-423 | I.2A′ | CH₂-c-C₃H₅ | CH₂CH₃ | 2-F | H |
| A-424 | I.2A′ | CH₂CH═CHCl | CH₂CH₃ | 2-F | H |
| A-425 | I.2A′ | CH₂CN | CH₂CH₂CH₃ | 2-F | H |
| A-426 | I.2A′ | CH₂OCH₃ | CH₂CH₂CH₃ | 2-F | H |
| A-427 | I.2A′ | CH₂-c-C₃H₅ | CH₂CH₂CH₃ | 2-F | H |
| A-428 | I.2A′ | CH₂CH═CHCl | CH₂CH₂CH₃ | 2-F | H |
| A-429 | I.2A′ | CH₂CN | OH | 2-F | H |
| A-430 | I.2A′ | CH₂OCH₃ | OH | 2-F | H |
| A-431 | I.2A′ | CH₂-c-C₃H₅ | OH | 2-F | H |
| A-432 | I.2A′ | CH₂CH═CHCl | OH | 2-F | H |
| A-433 | I.2A′ | CH₂CN | CH₂OH | 2-F | H |
| A-434 | I.2A′ | CH₂OCH₃ | CH₂OH | 2-F | H |
| A-435 | I.2A′ | CH₂-c-C₃H₅ | CH₂OH | 2-F | H |
| A-436 | I.2A′ | CH₂CH═CHCl | CH₂OH | 2-F | H |
| A-437 | I.2A′ | CH₂CN | NH₂ | 2-F | H |
| A-438 | I.2A′ | CH₂OCH₃ | NH₂ | 2-F | H |
| A-439 | I.2A′ | CH₂-c-C₃H₅ | NH₂ | 2-F | H |
| A-440 | I.2A′ | CH₂CH═CHCl | NH₂ | 2-F | H |
| A-441 | I.2A′ | CH₂CN | COOCH₃ | 2-F | H |
| A-442 | I.2A′ | CH₂OCH₃ | COOCH₃ | 2-F | H |
| A-443 | I.2A′ | CH₂-c-C₃H₅ | COOCH₃ | 2-F | H |
| A-444 | I.2A′ | CH₂CH═CHCl | COOCH₃ | 2-F | H |
| A-445 | I.2A′ | CH₂CN | CH₂CH═CH₂ | 2-F | H |
| A-446 | I.2A′ | CH₂OCH₃ | CH₂CH═CH₂ | 2-F | H |
| A-447 | I.2A′ | CH₂-c-C₃H₅ | CH₂CH═CH₂ | 2-F | H |
| A-448 | I.2A′ | CH₂CH═CHCl | CH₂CH═CH₂ | 2-F | H |
| A-449 | I.2A′ | CH₂CN | CH₃ | 3-F | H |
| A-450 | I.2A′ | CH₂OCH₃ | CH₃ | 3-F | H |
| A-451 | I.2A′ | CH₂-c-C₃H₅ | CH₃ | 3-F | H |
| A-452 | I.2A′ | CH₂CH═CHCl | CH₃ | 3-F | H |
| A-453 | I.2A′ | CH₂CN | CH₂CH₃ | 3-F | H |
| A-454 | I.2A′ | CH₂OCH₃ | CH₂CH₃ | 3-F | H |
| A-455 | I.2A′ | CH₂-c-C₃H₅ | CH₂CH₃ | 3-F | H |
| A-456 | I.2A′ | CH₂CH═CHCl | CH₂CH₃ | 3-F | H |
| A-457 | I.2A′ | CH₂CN | CH₂CH₂CH₃ | 3-F | H |
| A-458 | I.2A′ | CH₂OCH₃ | CH₂CH₂CH₃ | 3-F | H |
| A-459 | I.2A′ | CH₂-c-C₃H₅ | CH₂CH₂CH₃ | 3-F | H |
| A-460 | I.2A′ | CH₂CH═CHCl | CH₂CH₂CH₃ | 3-F | H |
| A-461 | I.2A′ | CH₂CN | OH | 3-F | H |
| A-462 | I.2A′ | CH₂OCH₃ | OH | 3-F | H |
| A-463 | I.2A′ | CH₂-c-C₃H₅ | OH | 3-F | H |
| A-464 | I.2A′ | CH₂CH═CHCl | OH | 3-F | H |
| A-465 | I.2A′ | CH₂CN | CH₂OH | 3-F | H |
| A-466 | I.2A′ | CH₂OCH₃ | CH₂OH | 3-F | H |
| A-467 | I.2A′ | CH₂-c-C₃H₅ | CH₂OH | 3-F | H |
| A-468 | I.2A′ | CH₂CH═CHCl | CH₂OH | 3-F | H |
| A-469 | I.2A′ | CH₂CN | NH₂ | 3-F | H |
| A-470 | I.2A′ | CH₂OCH₃ | NH₂ | 3-F | H |
| A-471 | I.2A′ | CH₂-c-C₃H₅ | NH₂ | 3-F | H |
| A-472 | I.2A′ | CH₂CH═CHCl | NH₂ | 3-F | H |
| A-473 | I.2A′ | CH₂CN | COOCH₃ | 3-F | H |
| A-474 | I.2A′ | CH₂OCH₃ | COOCH₃ | 3-F | H |
| A-475 | I.2A′ | CH₂-c-C₃H₅ | COOCH₃ | 3-F | H |
| A-476 | I.2A′ | CH₂CH═CHCl | COOCH₃ | 3-F | H |
| A-477 | I.2A′ | CH₂CN | CH₂CH═CH₂ | 3-F | H |
| A-478 | I.2A′ | CH₂OCH₃ | CH₂CH═CH₂ | 3-F | H |
| A-479 | I.2A′ | CH₂-c-C₃H₅ | CH₂CH═CH₂ | 3-F | H |
| A-480 | I.2A′ | CH₂CH═CHCl | CH₂CH═CH₂ | 3-F | H |
| A-481 | I.2A′ | CH₂CN | CH₃ | 4-F | H |
| A-482 | I.2A′ | CH₂OCH₃ | CH₃ | 4-F | H |
| A-483 | I.2A′ | CH₂-c-C₃H₅ | CH₃ | 4-F | H |
| A-484 | I.2A′ | CH₂CH═CHCl | CH₃ | 4-F | H |
| A-485 | I.2A′ | CH₂CN | CH₂CH₃ | 4-F | H |
| A-486 | I.2A′ | CH₂OCH₃ | CH₂CH₃ | 4-F | H |
| A-487 | I.2A′ | CH₂-c-C₃H₅ | CH₂CH₃ | 4-F | H |
| A-488 | I.2A′ | CH₂CH═CHCl | CH₂CH₃ | 4-F | H |
| A-489 | I.2A′ | CH₂CN | CH₂CH₂CH₃ | 4-F | H |
| A-490 | I.2A′ | CH₂OCH₃ | CH₂CH₂CH₃ | 4-F | H |
| A-491 | I.2A′ | CH₂-c-C₃H₅ | CH₂CH₂CH₃ | 4-F | H |
| A-492 | I.2A′ | CH₂CH═CHCl | CH₂CH₂CH₃ | 4-F | H |
| A-493 | I.2A′ | CH₂CN | OH | 4-F | H |
| A-494 | I.2A′ | CH₂OCH₃ | OH | 4-F | H |
| A-495 | I.2A′ | CH₂-c-C₃H₅ | OH | 4-F | H |
| A-496 | I.2A′ | CH₂CH═CHCl | OH | 4-F | H |
| A-497 | I.2A′ | CH₂CN | CH₂OH | 4-F | H |
| A-498 | I.2A′ | CH₂OCH₃ | CH₂OH | 4-F | H |
| A-499 | I.2A′ | CH₂-c-C₃H₅ | CH₂OH | 4-F | H |
| A-500 | I.2A′ | CH₂CH═CHCl | CH₂OH | 4-F | H |
| A-501 | I.2A′ | CH₂CN | NH₂ | 4-F | H |
| A-502 | I.2A′ | CH₂OCH₃ | NH₂ | 4-F | H |
| A-503 | I.2A′ | CH₂-c-C₃H₅ | NH₂ | 4-F | H |
| A-504 | I.2A′ | CH₂CH═CHCl | NH₂ | 4-F | H |
| A-505 | I.2A′ | CH₂CN | COOCH₃ | 4-F | H |
| A-506 | I.2A′ | CH₂OCH₃ | COOCH₃ | 4-F | H |
| A-507 | I.2A′ | CH₂-c-C₃H₅ | COOCH₃ | 4-F | H |
| A-508 | I.2A′ | CH₂CH═CHCl | COOCH₃ | 4-F | H |
| A-509 | I.2A′ | CH₂CN | CH₂CH═CH₂ | 4-F | H |
| A-510 | I.2A′ | CH₂OCH₃ | CH₂CH═CH₂ | 4-F | H |
| A-511 | I.2A′ | CH₂-c-C₃H₅ | CH₂CH═CH₂ | 4-F | H |
| A-512 | I.2A′ | CH₂CH═CHCl | CH₂CH═CH₂ | 4-F | H |
| A-513 | I.2A′ | CH₂CN | CH₃ | 3-F | 2-F |
| A-514 | I.2A′ | CH₂OCH₃ | CH₃ | 3-F | 2-F |
| A-515 | I.2A′ | CH₂-c-C₃H₅ | CH₃ | 3-F | 2-F |
| A-516 | I.2A′ | CH₂CH═CHCl | CH₃ | 3-F | 2-F |
| A-517 | I.2A′ | CH₂CN | CH₂CH₃ | 3-F | 2-F |
| A-518 | I.2A′ | CH₂OCH₃ | CH₂CH₃ | 3-F | 2-F |
| A-519 | I.2A′ | CH₂-c-C₃H₅ | CH₂CH₃ | 3-F | 2-F |
| A-520 | I.2A′ | CH₂CH═CHCl | CH₂CH₃ | 3-F | 2-F |
| A-521 | I.2A′ | CH₂CN | CH₂CH₂CH₃ | 3-F | 2-F |
| A-522 | I.2A′ | CH₂OCH₃ | CH₂CH₂CH₃ | 3-F | 2-F |
| A-523 | I.2A′ | CH₂-c-C₃H₅ | CH₂CH₂CH₃ | 3-F | 2-F |
| A-524 | I.2A′ | CH₂CH═CHCl | CH₂CH₂CH₃ | 3-F | 2-F |
| A-525 | I.2A′ | CH₂CN | OH | 3-F | 2-F |
| A-526 | I.2A′ | CH₂OCH₃ | OH | 3-F | 2-F |
| A-527 | I.2A′ | CH₂-c-C₃H₅ | OH | 3-F | 2-F |
| A-528 | I.2A′ | CH₂CH═CHCl | OH | 3-F | 2-F |
| A-529 | I.2A′ | CH₂CN | CH₂OH | 3-F | 2-F |
| A-530 | I.2A′ | CH₂OCH₃ | CH₂OH | 3-F | 2-F |
| A-531 | I.2A′ | CH₂-c-C₃H₅ | CH₂OH | 3-F | 2-F |
| A-532 | I.2A′ | CH₂CH═CHCl | CH₂OH | 3-F | 2-F |
| A-533 | I.2A′ | CH₂CN | NH₂ | 3-F | 2-F |
| A-534 | I.2A′ | CH₂OCH₃ | NH₂ | 3-F | 2-F |
| A-535 | I.2A′ | CH₂-c-C₃H₅ | NH₂ | 3-F | 2-F |
| A-536 | I.2A′ | CH₂CH═CHCl | NH₂ | 3-F | 2-F |
| A-537 | I.2A′ | CH₂CN | COOCH₃ | 3-F | 2-F |
| A-538 | I.2A′ | CH₂OCH₃ | COOCH₃ | 3-F | 2-F |
| A-539 | I.2A′ | CH₂-c-C₃H₅ | COOCH₃ | 3-F | 2-F |
| A-540 | I.2A′ | CH₂CH═CHCl | COOCH₃ | 3-F | 2-F |
| A-541 | I.2A′ | CH₂CN | CH₂CH═CH₂ | 3-F | 2-F |
| A-542 | I.2A′ | CH₂OCH₃ | CH₂CH═CH₂ | 3-F | 2-F |
| A-543 | I.2A′ | CH₂-c-C₃H₅ | CH₂CH═CH₂ | 3-F | 2-F |
| A-544 | I.2A′ | CH₂CH═CHCl | CH₂CH═CH₂ | 3-F | 2-F |
| A-545 | I.2A′ | CH₂CN | CH₃ | 3-F | 4-F |
| A-546 | I.2A′ | CH₂OCH₃ | CH₃ | 3-F | 4-F |
| A-547 | I.2A′ | CH₂-c-C₃H₅ | CH₃ | 3-F | 4-F |
| A-548 | I.2A′ | CH₂CH═CHCl | CH₃ | 3-F | 4-F |
| A-549 | I.2A′ | CH₂CN | CH₂CH₃ | 3-F | 4-F |
| A-550 | I.2A′ | CH₂OCH₃ | CH₂CH₃ | 3-F | 4-F |
| A-551 | I.2A′ | CH₂-c-C₃H₅ | CH₂CH₃ | 3-F | 4-F |
| A-552 | I.2A′ | CH₂CH═CHCl | CH₂CH₃ | 3-F | 4-F |
| A-553 | I.2A′ | CH₂CN | CH₂CH₂CH₃ | 3-F | 4-F |
| A-554 | I.2A′ | CH₂OCH₃ | CH₂CH₂CH₃ | 3-F | 4-F |
| A-555 | I.2A′ | CH₂-c-C₃H₅ | CH₂CH₂CH₃ | 3-F | 4-F |
| A-556 | I.2A′ | CH₂CH═CHCl | CH₂CH₂CH₃ | 3-F | 4-F |
| A-557 | I.2A′ | CH₂CN | OH | 3-F | 4-F |
| A-558 | I.2A′ | CH₂OCH₃ | OH | 3-F | 4-F |
| A-559 | I.2A′ | CH₂-c-C₃H₅ | OH | 3-F | 4-F |
| A-560 | I.2A′ | CH₂CH═CHCl | OH | 3-F | 4-F |
| A-561 | I.2A′ | CH₂CN | CH₂OH | 3-F | 4-F |
| A-562 | I.2A′ | CH₂OCH₃ | CH₂OH | 3-F | 4-F |
| A-563 | I.2A′ | CH₂-c-C₃H₅ | CH₂OH | 3-F | 4-F |
| A-564 | I.2A′ | CH₂CH═CHCl | CH₂OH | 3-F | 4-F |
| A-565 | I.2A′ | CH₂CN | NH₂ | 3-F | 4-F |
| A-566 | I.2A′ | CH₂OCH₃ | NH₂ | 3-F | 4-F |
| A-567 | I.2A′ | CH₂-c-C₃H₅ | NH₂ | 3-F | 4-F |
| A-568 | I.2A′ | CH₂CH═CHCl | NH₂ | 3-F | 4-F |
| A-569 | I.2A′ | CH₂CN | COOCH₃ | 3-F | 4-F |
| A-570 | I.2A′ | CH₂OCH₃ | COOCH₃ | 3-F | 4-F |
| A-571 | I.2A′ | CH₂-c-C₃H₅ | COOCH₃ | 3-F | 4-F |
| A-572 | I.2A′ | CH₂CH═CHCl | COOCH₃ | 3-F | 4-F |
| A-573 | I.2A′ | CH₂CN | CH₂CH═CH₂ | 3-F | 4-F |
| A-574 | I.2A′ | CH₂OCH₃ | CH₂CH═CH₂ | 3-F | 4-F |
| A-575 | I.2A′ | CH₂-c-C₃H₅ | CH₂CH═CH₂ | 3-F | 4-F |
| A-576 | I.2A′ | CH₂CH═CHCl | CH₂CH═CH₂ | 3-F | 4-F |
| A-577 | I.2B′ | CH₂CN | CH₃ | H | H |
| A-578 | I.2B′ | CH₂OCH₃ | CH₃ | H | H |
| A-579 | I.2B′ | CH₂-c-C₃H₅ | CH₃ | H | H |
| A-580 | I.2B′ | CH₂CH═CHCl | CH₃ | H | H |
| A-581 | I.2B′ | CH₂CN | CH₂CH₃ | H | H |
| A-582 | I.2B′ | CH₂OCH₃ | CH₂CH₃ | H | H |
| A-583 | I.2B′ | CH₂-c-C₃H₅ | CH₂CH₃ | H | H |
| A-584 | I.2B′ | CH₂CH═CHCl | CH₂CH₃ | H | H |
| A-585 | I.2B′ | CH₂CN | CH₂CH₂CH₃ | H | H |
| A-586 | I.2B′ | CH₂OCH₃ | CH₂CH₂CH₃ | H | H |
| A-587 | I.2B′ | CH₂-c-C₃H₅ | CH₂CH₂CH₃ | H | H |
| A-588 | I.2B′ | CH₂CH═CHCl | CH₂CH₂CH₃ | H | H |
| A-589 | I.2B′ | CH₂CN | OH | H | H |
| A-590 | I.2B′ | CH₂OCH₃ | OH | H | H |
| A-591 | I.2B′ | CH₂-c-C₃H₅ | OH | H | H |
| A-592 | I.2B′ | CH₂CH═CHCl | OH | H | H |
| A-593 | I.2B′ | CH₂CN | CH₂OH | H | H |
| A-594 | I.2B′ | CH₂OCH₃ | CH₂OH | H | H |
| A-595 | I.2B′ | CH₂-c-C₃H₅ | CH₂OH | H | H |
| A-596 | I.2B′ | CH₂CH═CHCl | CH₂OH | H | H |
| A-597 | I.2B′ | CH₂CN | NH₂ | H | H |
| A-598 | I.2B′ | CH₂OCH₃ | NH₂ | H | H |
| A-599 | I.2B′ | CH₂-c-C₃H₅ | NH₂ | H | H |
| A-600 | I.2B′ | CH₂CH═CHCl | NH₂ | H | H |
| A-601 | I.2B′ | CH₂CN | COOCH₃ | H | H |
| A-602 | I.2B′ | CH₂OCH₃ | COOCH₃ | H | H |
| A-603 | I.2B′ | CH₂-c-C₃H₅ | COOCH₃ | H | H |
| A-604 | I.2B′ | CH₂CH═CHCl | COOCH₃ | H | H |
| A-605 | I.2B′ | CH₂CN | CH₂CH═CH₂ | H | H |
| A-606 | I.2B′ | CH₂OCH₃ | CH₂CH═CH₂ | H | H |
| A-607 | I.2B′ | CH₂-c-C₃H₅ | CH₂CH═CH₂ | H | H |
| A-608 | I.2B′ | CH₂CH═CHCl | CH₂CH═CH₂ | H | H |
| A-609 | I.2B′ | CH₂CN | CH₃ | H | H |
| A-610 | I.2B′ | CH₂OCH₃ | CH₃ | 2-F | H |
| A-611 | I.2B′ | CH₂-c-C₃H₅ | CH₃ | 2-F | H |
| A-612 | I.2B′ | CH₂CH═CHCl | CH₃ | 2-F | H |
| A-613 | I.2B′ | CH₂CN | CH₂CH₃ | 2-F | H |
| A-614 | I.2B′ | CH₂OCH₃ | CH₂CH₃ | 2-F | H |
| A-615 | I.2B′ | CH₂-c-C₃H₅ | CH₂CH₃ | 2-F | H |
| A-616 | I.2B′ | CH₂CH═CHCl | CH₂CH₃ | 2-F | H |
| A-617 | I.2B′ | CH₂CN | CH₂CH₂CH₃ | 2-F | H |
| A-618 | I.2B′ | CH₂OCH₃ | CH₂CH₂CH₃ | 2-F | H |
| A-619 | I.2B′ | CH₂-c-C₃H₅ | CH₂CH₂CH₃ | 2-F | H |
| A-620 | I.2B′ | CH₂CH═CHCl | CH₂CH₂CH₃ | 2-F | H |
| A-621 | I.2B′ | CH₂CN | OH | 2-F | H |
| A-622 | I.2B′ | CH₂OCH₃ | OH | 2-F | H |
| A-623 | I.2B′ | CH₂-c-C₃H₅ | OH | 2-F | H |
| A-624 | I.2B′ | CH₂CH═CHCl | OH | 2-F | H |
| A-625 | I.2B′ | CH₂CN | CH₂OH | 2-F | H |
| A-626 | I.2B′ | CH₂OCH₃ | CH₂OH | 2-F | H |
| A-627 | I.2B′ | CH₂-c-C₃H₅ | CH₂OH | 2-F | H |
| A-628 | I.2B′ | CH₂CH═CHCl | CH₂OH | 2-F | H |
| A-629 | I.2B′ | CH₂CN | NH₂ | 2-F | H |
| A-630 | I.2B′ | CH₂OCH₃ | NH₂ | 2-F | H |
| A-631 | I.2B′ | CH₂-c-C₃H₅ | NH₂ | 2-F | H |
| A-632 | I.2B′ | CH₂CH═CHCl | NH₂ | 2-F | H |
| A-633 | I.2B′ | CH₂CN | COOCH₃ | 2-F | H |
| A-634 | I.2B′ | CH₂OCH₃ | COOCH₃ | 2-F | H |
| A-635 | I.2B′ | CH₂-c-C₃H₅ | COOCH₃ | 2-F | H |
| A-636 | I.2B′ | CH₂CH═CHCl | COOCH₃ | 2-F | H |
| A-637 | I.2B′ | CH₂CN | CH₂CH═CH₂ | 2-F | H |
| A-638 | I.2B′ | CH₂OCH₃ | CH₂CH═CH₂ | 2-F | H |
| A-639 | I.2B′ | CH₂-c-C₃H₅ | CH₂CH═CH₂ | 2-F | H |
| A-640 | I.2B′ | CH₂CH═CHCl | CH₂CH═CH₂ | 2-F | H |
| A-641 | I.2B′ | CH₂CN | CH₃ | 3-F | H |
| A-642 | I.2B′ | CH₂OCH₃ | CH₃ | 3-F | H |
| A-643 | I.2B′ | CH₂-c-C₃H₅ | CH₃ | 3-F | H |
| A-644 | I.2B′ | CH₂CH═CHCl | CH₃ | 3-F | H |
| A-645 | I.2B′ | CH₂CN | CH₂CH₃ | 3-F | H |
| A-646 | I.2B′ | CH₂OCH₃ | CH₂CH₃ | 3-F | H |
| A-647 | I.2B′ | CH₂-c-C₃H₅ | CH₂CH₃ | 3-F | H |
| A-648 | I.2B′ | CH₂CH═CHCl | CH₂CH₃ | 3-F | H |
| A-649 | I.2B′ | CH₂CN | CH₂CH₂CH₃ | 3-F | H |
| A-650 | I.2B′ | CH₂OCH₃ | CH₂CH₂CH₃ | 3-F | H |
| A-651 | I.2B′ | CH₂-c-C₃H₅ | CH₂CH₂CH₃ | 3-F | H |
| A-652 | I.2B′ | CH₂CH═CHCl | CH₂CH₂CH₃ | 3-F | H |
| A-653 | I.2B′ | CH₂CN | OH | 3-F | H |
| A-654 | I.2B′ | CH₂OCH₃ | OH | 3-F | H |
| A-655 | I.2B′ | CH₂-c-C₃H₅ | OH | 3-F | H |
| A-656 | I.2B′ | CH₂CH═CHCl | OH | 3-F | H |
| A-657 | I.2B′ | CH₂CN | CH₂OH | 3-F | H |
| A-658 | I.2B′ | CH₂OCH₃ | CH₂OH | 3-F | H |
| A-659 | I.2B′ | CH₂-c-C₃H₅ | CH₂OH | 3-F | H |
| A-660 | I.2B′ | CH₂CH═CHCl | CH₂OH | 3-F | H |
| A-661 | I.2B′ | CH₂CN | NH₂ | 3-F | H |
| A-662 | I.2B′ | CH₂OCH₃ | NH₂ | 3-F | H |
| A-663 | I.2B′ | CH₂-c-C₃H₅ | NH₂ | 3-F | H |
| A-664 | I.2B′ | CH₂CH═CHCl | NH₂ | 3-F | H |
| A-665 | I.2B′ | CH₂CN | COOCH₃ | 3-F | H |
| A-666 | I.2B′ | CH₂OCH₃ | COOCH₃ | 3-F | H |
| A-667 | I.2B′ | CH₂-c-C₃H₅ | COOCH₃ | 3-F | H |
| A-668 | I.2B′ | CH₂CH═CHCl | COOCH₃ | 3-F | H |
| A-669 | I.2B′ | CH₂CN | CH₂CH═CH₂ | 3-F | H |
| A-670 | I.2B′ | CH₂OCH₃ | CH₂CH═CH₂ | 3-F | H |
| A-671 | I.2B′ | CH₂-c-C₃H₅ | CH₂CH═CH₂ | 3-F | H |
| A-672 | I.2B′ | CH₂CH═CHCl | CH₂CH═CH₂ | 3-F | H |
| A-673 | I.2B′ | CH₂CN | CH₃ | 4-F | H |
| A-674 | I.2B′ | CH₂OCH₃ | CH₃ | 4-F | H |
| A-675 | I.2B′ | CH₂-c-C₃H₅ | CH₃ | 4-F | H |
| A-676 | I.2B′ | CH₂CH═CHCl | CH₃ | 4-F | H |
| A-677 | I.2B′ | CH₂CN | CH₂CH₃ | 4-F | H |
| A-678 | I.2B′ | CH₂OCH₃ | CH₂CH₃ | 4-F | H |
| A-679 | I.2B′ | CH₂-c-C₃H₅ | CH₂CH₃ | 4-F | H |
| A-680 | I.2B′ | CH₂CH═CHCl | CH₂CH₃ | 4-F | H |
| A-681 | I.2B′ | CH₂CN | CH₂CH₂CH₃ | 4-F | H |
| A-682 | I.2B′ | CH₂OCH₃ | CH₂CH₂CH₃ | 4-F | H |
| A-683 | I.2B′ | CH₂-c-C₃H₅ | CH₂CH₂CH₃ | 4-F | H |
| A-684 | I.2B′ | CH₂CH═CHCl | CH₂CH₂CH₃ | 4-F | H |
| A-685 | I.2B′ | CH₂CN | OH | 4-F | H |
| A-686 | I.2B′ | CH₂OCH₃ | OH | 4-F | H |
| A-687 | I.2B′ | CH₂-c-C₃H₅ | OH | 4-F | H |
| A-688 | I.2B′ | CH₂CH═CHCl | OH | 4-F | H |
| A-689 | I.2B′ | CH₂CN | CH₂OH | 4-F | H |
| A-690 | I.2B′ | CH₂OCH₃ | CH₂OH | 4-F | H |
| A-691 | I.2B′ | CH₂-c-C₃H₅ | CH₂OH | 4-F | H |
| A-692 | I.2B′ | CH₂CH═CHCl | CH₂OH | 4-F | H |
| A-693 | I.2B′ | CH₂CN | NH₂ | 4-F | H |
| A-694 | I.2B′ | CH₂OCH₃ | NH₂ | 4-F | H |
| A-695 | I.2B′ | CH₂-c-C₃H₅ | NH₂ | 4-F | H |
| A-696 | I.2B′ | CH₂CH═CHCl | NH₂ | 4-F | H |
| A-697 | I.2B′ | CH₂CN | COOCH₃ | 4-F | H |
| A-698 | I.2B′ | CH₂OCH₃ | COOCH₃ | 4-F | H |
| A-699 | I.2B′ | CH₂-c-C₃H₅ | COOCH₃ | 4-F | H |
| A-700 | I.2B′ | CH₂CH═CHCl | COOCH₃ | 4-F | H |
| A-701 | I.2B′ | CH₂CN | CH₂CH═CH₂ | 4-F | H |
| A-702 | I.2B′ | CH₂OCH₃ | CH₂CH═CH₂ | 4-F | H |
| A-703 | I.2B′ | CH₂-c-C₃H₅ | CH₂CH═CH₂ | 4-F | H |
| A-704 | I.2B′ | CH₂CH═CHCl | CH₂CH═CH₂ | 4-F | H |
| A-705 | I.2B′ | CH₂CN | CH₃ | 3-F | 2-F |
| A-706 | I.2B′ | CH₂OCH₃ | CH₃ | 3-F | 2-F |
| A-707 | I.2B′ | CH₂-c-C₃H₅ | CH₃ | 3-F | 2-F |
| A-708 | I.2B′ | CH₂CH═CHCl | CH₃ | 3-F | 2-F |
| A-709 | I.2B′ | CH₂CN | CH₂CH₃ | 3-F | 2-F |
| A-710 | I.2B′ | CH₂OCH₃ | CH₂CH₃ | 3-F | 2-F |
| A-711 | I.2B′ | CH₂-c-C₃H₅ | CH₂CH₃ | 3-F | 2-F |
| A-712 | I.2B′ | CH₂CH═CHCl | CH₂CH₃ | 3-F | 2-F |
| A-713 | I.2B′ | CH₂CN | CH₂CH₂CH₃ | 3-F | 2-F |
| A-714 | I.2B′ | CH₂OCH₃ | CH₂CH₂CH₃ | 3-F | 2-F |
| A-715 | I.2B′ | CH₂-c-C₃H₅ | CH₂CH₂CH₃ | 3-F | 2-F |
| A-716 | I.2B′ | CH₂CH═CHCl | CH₂CH₂CH₃ | 3-F | 2-F |
| A-717 | I.2B′ | CH₂CN | OH | 3-F | 2-F |
| A-718 | I.2B′ | CH₂OCH₃ | OH | 3-F | 2-F |
| A-719 | I.2B′ | CH₂-c-C₃H₅ | OH | 3-F | 2-F |
| A-720 | I.2B′ | CH₂CH═CHCl | OH | 3-F | 2-F |
| A-721 | I.2B′ | CH₂CN | CH₂OH | 3-F | 2-F |
| A-722 | I.2B′ | CH₂OCH₃ | CH₂OH | 3-F | 2-F |
| A-723 | I.2B′ | CH₂-c-C₃H₅ | CH₂OH | 3-F | 2-F |
| A-724 | I.2B′ | CH₂CH═CHCl | CH₂OH | 3-F | 2-F |
| A-725 | I.2B′ | CH₂CN | NH₂ | 3-F | 2-F |
| A-726 | I.2B′ | CH₂OCH₃ | NH₂ | 3-F | 2-F |
| A-727 | I.2B′ | CH₂-c-C₃H₅ | NH₂ | 3-F | 2-F |
| A-728 | I.2B′ | CH₂CH═CHCl | NH₂ | 3-F | 2-F |
| A-729 | I.2B′ | CH₂CN | COOCH₃ | 3-F | 2-F |
| A-730 | I.2B′ | CH₂OCH₃ | COOCH₃ | 3-F | 2-F |
| A-731 | I.2B′ | CH₂-c-C₃H₅ | COOCH₃ | 3-F | 2-F |
| A-732 | I.2B′ | CH₂CH═CHCl | COOCH₃ | 3-F | 2-F |
| A-733 | I.2B′ | CH₂CN | CH₂CH═CH₂ | 3-F | 2-F |
| A-734 | I.2B′ | CH₂OCH₃ | CH₂CH═CH₂ | 3-F | 2-F |
| A-735 | I.2B′ | CH₂-c-C₃H₅ | CH₂CH═CH₂ | 3-F | 2-F |
| A-736 | I.2B′ | CH₂CH═CHCl | CH₂CH═CH₂ | 3-F | 2-F |
| A-737 | I.2B′ | CH₂CN | CH₃ | 3-F | 4-F |
| A-738 | I.2B′ | CH₂OCH₃ | CH₃ | 3-F | 4-F |
| A-739 | I.2B′ | CH₂-c-C₃H₅ | CH₃ | 3-F | 4-F |
| A-740 | I.2B′ | CH₂CH═CHCl | CH₃ | 3-F | 4-F |
| A-741 | I.2B′ | CH₂CN | CH₂CH₃ | 3-F | 4-F |
| A-742 | I.2B′ | CH₂OCH₃ | CH₂CH₃ | 3-F | 4-F |
| A-743 | I.2B′ | CH₂-c-C₃H₅ | CH₂CH₃ | 3-F | 4-F |
| A-744 | I.2B′ | CH₂CH═CHCl | CH₂CH₃ | 3-F | 4-F |
| A-745 | I.2B′ | CH₂CN | CH₂CH₂CH₃ | 3-F | 4-F |
| A-746 | I.2B′ | CH₂OCH₃ | CH₂CH₂CH₃ | 3-F | 4-F |
| A-747 | I.2B′ | CH₂-c-C₃H₅ | CH₂CH₂CH₃ | 3-F | 4-F |
| A-748 | I.2B′ | CH₂CH═CHCl | CH₂CH₂CH₃ | 3-F | 4-F |
| A-749 | I.2B′ | CH₂CN | OH | 3-F | 4-F |
| A-750 | I.2B′ | CH₂OCH₃ | OH | 3-F | 4-F |
| A-751 | I.2B′ | CH₂-c-C₃H₅ | OH | 3-F | 4-F |
| A-752 | I.2B′ | CH₂CH═CHCl | OH | 3-F | 4-F |
| A-753 | I.2B′ | CH₂CN | CH₂OH | 3-F | 4-F |
| A-754 | I.2B′ | CH₂OCH₃ | CH₂OH | 3-F | 4-F |
| A-755 | I.2B′ | CH₂-c-C₃H₅ | CH₂OH | 3-F | 4-F |
| A-756 | I.2B′ | CH₂CH═CHCl | CH₂OH | 3-F | 4-F |
| A-757 | I.2B′ | CH₂CN | NH₂ | 3-F | 4-F |
| A-758 | I.2B′ | CH₂OCH₃ | NH₂ | 3-F | 4-F |
| A-759 | I.2B′ | CH₂-c-C₃H₅ | NH₂ | 3-F | 4-F |
| A-760 | I.2B′ | CH₂CH═CHCl | NH₂ | 3-F | 4-F |
| A-761 | I.2B′ | CH₂CN | COOCH₃ | 3-F | 4-F |
| A-762 | I.2B′ | CH₂OCH₃ | COOCH₃ | 3-F | 4-F |
| A-763 | I.2B′ | CH₂-c-C₃H₅ | COOCH₃ | 3-F | 4-F |
| A-764 | I.2B′ | CH₂CH═CHCl | COOCH₃ | 3-F | 4-F |
| A-765 | I.2B′ | CH₂CN | CH₂CH═CH₂ | 3-F | 4-F |
| A-766 | I.2B′ | CH₂OCH₃ | CH₂CH═CH₂ | 3-F | 4-F |
| A-767 | I.2B′ | CH₂-c-C₃H₅ | CH₂CH═CH₂ | 3-F | 4-F |
| A-768 | I.2B′ | CH₂CH═CHCl | CH₂CH═CH₂ | 3-F | 4-F |

c-C₃H₅ = cyclopropyl

From among the compounds mentioned above in an exemplary manner and their salts, preference is given to those compounds and salts in which the exo double bond at the piperazine ring has the (Z) configuration. Preference is also given to mixtures of the (E) isomer with the (Z) isomer in which the Z isomer is present in excess, in particular to isomer mixtures having an E/Z ratio of not more than 1:2, in particular not more than 1:5.

From among the compounds of the formula I.1B′ mentioned above in an exemplary manner and their salts, preference is given to those compounds and salts in which R³ and the hydrogen atom in position R⁴ have a cis configuration. Preference is also given to mixtures of the cis and trans isomers in which the cis isomer is present in excess, in particular to isomer mixtures having a cis/trans ratio of not more than 1:2, in particular not more than 1:5.

From among the compounds mentioned here in an exemplary manner and their salts, preference is given to those compounds and salts in which the carbon atom which carries the group R³ has the S configuration, and also to enantiomer mixtures having an enantiomeric excess of the S enantiomer, in particular those having an ee value of at least 70%, particularly preferably at least 80% and preferably at least 90%. Preference is also given to the racemates of these compounds and their salts.

The compounds I and their agriculturally useful salts are suitable, both as isomer mixtures and in the form of the pure isomers, as herbicides. They are suitable as such or as an appropriately formulated composition. The herbicidal compositions comprising the compound I, in particular the preferred aspects thereof, control vegetation on non-crop areas very efficiently, especially at high rates of application. They act against broad-leaved weeds and weed grasses in crops such as wheat, rice, corn, soybeans and cotton without causing any significant damage to the crop plants. This effect is mainly observed at low rates of application.

Depending on the application method in question, the compounds I, in particular the preferred aspects thereof, or compositions comprising them can additionally be employed in a further number of crop plants for eliminating unwanted plants. Examples of suitable crops are the following: Allium cepa, Ananas comosus, Arachis hypogaea, Asparagus officinalis, Avena sativa, Beta vulgaris spec. altissima, Beta vulgaris spec. rapa, Brassica napus var. napus, Brassica napus var. napobrassica, Brassica rapa var. silvestris, Brassica oleracea, Brassica nigra, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pistacia vera, Pisum sativum, Prunus avium, Prunus persica, Pyrus communis, Prunus armeniaca, Prunus cerasus, Prunus dulcis and Prunus domestica, Ribes sylvestre, Ricinus communis, Saccharum officinarum, Secale cereale, Sinapis alba, Solanum tuberosum, Sorghum bicolor (S. vulgare), Theobroma cacao, Trifolium pratense, Triticum aestivum, Triticale, Triticum durum, Vicia faba, Vitis vinifera, Zea mays.

The term “crop plants” also includes plants which have been modified by breeding, mutagenesis or genetic engineering. Genetically modified plants are plants whose genetic material has been modified in a manner which does not occur under natural conditions by crossing, mutations or natural recombination (i.e. reassembly of the genetic information). Here, in general, one or more genes are integrated into the genetic material of the plant to improve the properties of the plant.

Accordingly, the term “crop plants” also includes plants which, by breeding and genetic engineering, have acquired tolerance to certain classes of herbicides, such as hydroxyphenylpyruvate dioxygenase (HPPD) inhibitors, acetolactate synthase (ALS) inhibitors, such as, for example, sulfonylureas (EP-A-0257993, U.S. Pat. No. 5,013,659) or imidazolinones (see, for example, U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073), enolpyruvylshikimate 3-phosphate synthase (EPSPS) inhibitors, such as, for example, glyphosate (see, for example, WO 92/00377), glutamine synthetase (GS) inhibitors, such as, for example, glufosinate (see, for example, EP-A-0242236, EP-A-242246), or oxynil herbicides (see, for example, U.S. Pat. No. 5,559,024).

Numerous crop plants, for example Clearfield® oilseed rape, tolerant to imidazolinones, for example imazamox, have been generated with the aid of classic breeding methods (mutagenesis). Crop plants such as soybeans, cotton, corn, beet and oilseed rape, resistant to glyphosate or glufosinate, which are available under the tradenames RoundupReady® (glyphosate) and Liberty Link® (glufosinate) have been generated with the aid of genetic engineering methods.

Accordingly, the term “crop plants” also includes plants which, with the aid of genetic engineering, produce one or more toxins, for example those of the bacterial strain Bacillus ssp. Toxins which are produced by such genetically modified plants include, for example, insecticidal proteins of Bacillus spp., in particular B. thuringiensis, such as the endotoxins Cry1Ab, Cry1Ac, Cry1F, Cry1Fa2, Cry2Ab, Cry3A, Cry3Bb1, Cry9c, Cry34Ab1 or Cry35Ab1; or vegetative insecticidal proteins (VIPs), for example VIP1, VIP2, VIP3, or VIP3A; insecticidal proteins of nematode-colonizing bacteria, for example Photorhabdus spp. or Xenorhabdus spp.; toxins of animal organisms, for example wasp, spider or scorpion toxins; fungal toxins, for example from Streptomycetes; plant lectins, for example from peas or barley; agglutinins; proteinase inhibitors, for example trypsin inhibitors, serine protease inhibitors, patatin, cystatin or papain inhibitors, ribosome-inactivating proteins (RIPs), for example ricin, corn-RIP, abrin, luffin, saporin or bryodin; steroid-metabolizing enzymes, for example 3-hydroxysteroid oxidase, ecdysteroid-IDP glycosyl transferase, cholesterol oxidase, ecdysone inhibitors, or HMG-CoA reductase; ion channel blockers, for example inhibitors of sodium channels or calcium channels; juvenile hormone esterase; receptors of the diuretic hormone (helicokinin receptors); stilbene synthase, bibenzyl synthase, chitinases and glucanases. In the plants, these toxins may also be produced as pretoxins, hybrid proteins or truncated or otherwise modified proteins. Hybrid proteins are characterized by a novel combination of different protein domains (see, for example, WO 2002/015701). Further examples of such toxins or genetically modified plants which produce these toxins are disclosed in EP-A 374 753, WO 93/007278, WO 95/34656, EP-A 427 529, EP-A 451 878, WO 03/018810 and WO 03/052073. The methods for producing these genetically modified plants are known to the person skilled in the art and disclosed, for example, in the publications mentioned above. Numerous of the toxins mentioned above bestow, upon the plants by which they are produced, tolerance to pests from all taxonomic classes of arthropods, in particular to beetles (Coeleropta), dipterans (Diptera) and butterflies (Lepidoptera) and to nematodes (Nematoda).

Genetically modified plants which produce one or more genes coding for insecticidal toxins are described, for example, in the publications mentioned above, and some of them are commercially available, such as, for example, YieldGard® (corn varieties producing the toxin Cry1Ab), YieldGard® Plus (corn varieties which produce the toxins Cry1Ab and Cry3Bb1), Starlink® (corn varieties which produce the toxin Cry9c), Herculex® RW (corn varieties which produce the toxins Cry34Ab1, Cry35Ab1 and the enzyme phosphinothricin-N-acetyltransferase [PAT]); NuCOTN® 33B (cotton varieties which produce the toxin Cry1Ac), Bollgard® I (cotton varieties which produce the toxin Cry1Ac), Bollgard® II (cotton varieties which produce the toxins Cry1Ac and Cry2Ab2); VIPCOT® (cotton varieties which produce a VIP toxin); NewLeaf® (potato varieties which produce the toxin Cry3A); Bt-Xtra®, NatureGard®, KnockOut®, BiteGard®, Protecta®, Bt11 (for example Agrisure® CB) and Bt176 from Syngenta Seeds SAS, France (corn varieties which produce the toxin Cry1Ab and the PAT enyzme), MIR604 from Syngenta Seeds SAS, France (corn varieties which produce a modified version of the toxin Cry3A, see WO 03/018810), MON 863 from Monsanto Europe S.A., Belgium (corn varieties which produce the toxin Cry3Bb1), IPC 531 from Monsanto Europe S.A., Belgium (cotton varieties which produce a modified version of the toxin Cry1Ac) and 1507 from Pioneer Overseas Corporation, Belgium (corn varieties which produce the toxin Cry1F and the PAT enzyme).

Accordingly, the term “crop plants” also includes plants which, with the aid of genetic engineering, produce one or more proteins which are more robust or have increased resistance to bacterial, viral or fungal pathogens, such as, for example, pathogenesis-related proteins (PR proteins, see EP-A 0 392 225), resistance proteins (for example potato varieties producing two resistance genes against Phytophthora infestans from the wild Mexican potato Solanum bulbocastanum) or T4 lysozyme (for example potato cultivars which, by producing this protein, are resistant to bacteria such as Erwinia amylvora).

Accordingly, the term “crop plants” also includes plants whose productivity has been improved with the aid of genetic engineering methods, for example by enhancing the potential yield (for example biomass, grain yield, starch, oil or protein content), tolerance to drought, salt or other limiting environmental factors or resistance to pests and fungal, bacterial and viral pathogens.

The term “crop plants” also includes plants whose ingredients have been modified with the aid of genetic engineering methods in particular for improving human or animal diet, for example by oil plants producing health-promoting long-chain omega 3 fatty acids or monounsaturated omega 9 fatty acids (for example Nexera® oilseed rape).

The term “crop plants” also includes plants which have been modified with the aid of genetic engineering methods for improving the production of raw materials, for example by increasing the amylopectin content of potatoes (Amflora® potato).

Furthermore, it has been found that the compounds of the formula I are also suitable for the defoliation and/or desiccation of plant parts, for which crop plants such as cotton, potato, oilseed rape, sunflower, soybean or field beans, in particular cotton, are suitable. In this regard, there have been found compositions for the desiccation and/or defoliation of plants, processes for preparing these compositions and methods for desiccating and/or defoliating plants using the compounds of the formula I.

As desiccants, the compounds of the formula I are particularly suitable for desiccating the above-ground parts of crop plants such as potato, oilseed rape, sunflower and soybean, but also cereals. This makes possible the fully mechanical harvesting of these important crop plants.

Also of economic interest is to facilitate harvesting, which is made possible by concentrating within a certain period of time the dehiscence, or reduction of adhesion to the tree, in citrus fruit, olives and other species and varieties of pomaceous fruit, stone fruit and nuts. The same mechanism, i.e. the promotion of the development of abscission tissue between fruit part or leaf part and shoot part of the plants is also essential for the readily controllable defoliation of useful plants, in particular cotton.

Moreover, a shortening of the time interval in which the individual cotton plants mature leads to an increased fiber quality after harvesting.

The compounds I, or the herbicidal compositions comprising the compounds I, can be used, for example, in the form of ready-to-spray aqueous solutions, powders, suspensions, also highly concentrated aqueous, oily or other suspensions or dispersions, emulsions, oil dispersions, pastes, dusts, materials for broadcasting, or granules, by means of spraying, atomizing, dusting, spreading, watering or treatment of the seed or mixing with the seed. The use forms depend on the intended purpose; in each case, they should ensure the finest possible distribution of the active ingredients according to the invention.

The herbicidal compositions comprise a herbicidally effective amount of at least one compound of the formula I or an agriculturally useful salt of I, and auxiliaries which are customary for the formulation of crop protection agents.

Examples of auxiliaries customary for the formulation of crop protection agents are inert auxiliaries, solid carriers, surfactants (such as dispersants, protective colloids, emulsifiers, wetting agents and tackifiers), organic and inorganic thickeners, bactericides, antifreeze agents, antifoams, optionally colorants and, for seed formulations, adhesives.

Examples of thickeners (i.e. compounds which impart to the formulation modified flow properties, i.e. high viscosity in the state of rest and low viscosity in motion) are polysaccharides, such as xanthan gum (Kelzan® from Kelco), Rhodopol® 23 (Rhone Poulenc) or Veegum® (from R.T. Vanderbilt), and also organic and inorganic sheet minerals, such as Attaclay® (from Engelhardt).

Examples of antifoams are silicone emulsions (such as, for example, Silikon® SRE, Wacker or Rhodorsil® from Rhodia), long-chain alcohols, fatty acids, salts of fatty acids, organofluorine compounds and mixtures thereof.

Bactericides can be added for stabilizing the aqueous herbicidal formulation. Examples of bactericides are bactericides based on dichlorophen and benzyl alcohol hemiformal (Proxel® from ICI or Acticide® RS from Thor Chemie and Kathon® MK from Rohm & Haas), and also isothiazolinone derivates, such as alkylisothiazolinones and benzisothiazolinones (Acticide MBS from Thor Chemie).

Examples of antifreeze agents are ethylene glycol, propylene glycol, urea or glycerol.

Examples of colorants are both sparingly water-soluble pigments and water-soluble dyes. Examples which may be mentioned are the dyes known under the names Rhodamin B, C.I. Pigment Red 112 and C.I. Solvent Red 1, and also pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of adhesives are polyvinylpyrrolidone, polyvinyl acetate, polyvinyl alcohol and tylose.

Suitable inert auxiliaries are, for example, the following:

mineral oil fractions of medium to high boiling point, such as kerosene and diesel oil, furthermore coal tar oils and oils of vegetable or animal origin, aliphatic, cyclic and aromatic hydrocarbons, for example paraffin, tetrahydronaphthalene, alkylated naphthalenes and their derivatives, alkylated benzenes and their derivatives, alcohols such as methanol, ethanol, propanol, butanol and cyclohexanol, ketones such as cyclohexanone or strongly polar solvents, for example amines such as N-methylpyrrolidone, and water.

Solid carriers are mineral earths such as silicas, silica gels, silicates, talc, kaolin, limestone, lime, chalk, bole, loess, clay, dolomite, diatomaceous earth, calcium sulfate, magnesium sulfate and magnesium oxide, ground synthetic materials, fertilizers such as ammonium sulfate, ammonium phosphate, ammonium nitrate and ureas, and products of vegetable origin, such as cereal meal, tree bark meal, wood meal and nutshell meal, cellulose powders, or other solid carriers.

Suitable surfactants (adjuvants, wetting agents, tackifiers, dispersants and also emulsifiers) are the alkali metal salts, alkaline earth metal salts and ammonium salts of aromatic sulfonic acids, for example lignosulfonic acids (e.g. Borrespers-types, Borregaard), phenolsulfonic acids, naphthalenesulfonic acids (Morwet types, Akzo Nobel) and dibutylnaphthalenesulfonic acid (Nekal types, BASF SE), and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkylphenyl or tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors and proteins, denatured proteins, polysaccharides (e.g. methylcellulose), hydrophobically modified starches, polyvinyl alcohol (Mowiol types Clariant), polycarboxylates (BASF SE, Sokalan types), polyalkoxylates, polyvinylamine (BASF SE, Lupamine types), polyethyleneimine (BASF SE, Lupasol types), polyvinylpyrrolidone and copolymers thereof.

Powders, materials for broadcasting and dusts can be prepared by mixing or grinding the active ingredients together with a solid carrier.

Granules, for example coated granules, impregnated granules and homogeneous granules, can be prepared by binding the active ingredients to solid carriers.

Aqueous use forms can be prepared from emulsion concentrates, suspensions, pastes, wettable powders or water-dispersible granules by adding water. To prepare emulsions, pastes or oil dispersions, the compounds of the formula I or Ia, either as such or dissolved in an oil or solvent, can be homogenized in water by means of a wetting agent, tackifier, dispersant or emulsifier. Alternatively, it is also possible to prepare concentrates comprising active substance, wetting agent, tackifier, dispersant or emulsifier and, if desired, solvent or oil, which are suitable for dilution with water.

The concentrations of the compounds of the formula I in the ready-to-use preparations can be varied within wide ranges. In general, the formulations comprise from 0.001 to 98% by weight, preferably 0.01 to 95% by weight of at least one active compound. The active compounds are employed in a purity of from 90% to 100%, preferably 95% to 100% (according to NMR spectrum).

The compounds I of the invention can for example be formulated as follows:

1. Products for Dilution with Water

A Water-Soluble Concentrates

10 parts by weight of active compound are dissolved in 90 parts by weight of water or a water-soluble solvent. As an alternative, wetters or other adjuvants are added. The active compound dissolves upon dilution with water. This gives a formulation with an active compound content of 10% by weight.

B Dispersible Concentrates

20 parts by weight of active compound are dissolved in 70 parts by weight of cyclohexanone with addition of 10 parts by weight of a dispersant, for example polyvinylpyrrolidone. Dilution with water gives a dispersion. The active compound content is 20% by weight.

C Emulsifiable Concentrates

15 parts by weight of active compound are dissolved in 75 parts by weight of an organic solvent (e.g. alkylaromatics) with addition of calcium dodecylbenzenesulfonate and castor oil ethoxylate (in each case 5 parts by weight). Dilution with water gives an emulsion. The formulation has an active compound content of 15% by weight.

D Emulsions

25 parts by weight of active compound are dissolved in 35 parts by weight of an organic solvent (e.g. alkylaromatics) with addition of calcium dodecylbenzenesulfonate and castor oil ethoxylate (in each case 5 parts by weight). This mixture is introduced into 30 parts by weight of water by means of an emulsifier (e.g. Ultraturrax) and made into a homogeneous emulsion. Dilution with water gives an emulsion. The formulation has an active compound content of 25% by weight.

### E Suspensions

In an agitated ball mill, 20 parts by weight of active compound are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

F Water-dispersible granules and water-soluble granules

50 parts by weight of active compound are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

G Water-Dispersible Powders and Water-Soluble Powders

75 parts by weight of active compound are ground in a rotor-stator mill with addition of 25 parts by weight of dispersants, wetters and silica gel. Dilution with water gives a stable dispersion or solution of the active compound. The active compound content of the formulation is 75% by weight.

### H Gel Formulations

In a ball mill, 20 parts by weight of active compound, 10 parts by weight of dispersant, 1 part by weight of gelling agent and 70 parts by weight of water or of an organic solvent are ground to give a fine suspension. Dilution with water gives a stable suspension with active compound content of 20% by weight.

2. Products To be Applied Undiluted

I Dusts

5 parts by weight of active compound are ground finely and mixed intimately with 95 parts by weight of finely divided kaolin. This gives a dusting powder with an active compound content of 5% by weight.

J Granules (GR, FG, GG, MG)

0.5 parts by weight of active compound are ground finely and associated with 99.5 parts by weight of carriers. Current methods here are extrusion, spray-drying or the fluidized bed. This gives granules to be applied undiluted with an active compound content of 0.5% by weight.

### K ULV Solutions (UL)

10 parts by weight of active compound are dissolved in 90 parts by weight of an organic solvent, for example xylene. This gives a product to be applied undiluted with an active compound content of 10% by weight.

The compounds I or the herbicidal compositions comprising them can be applied pre- or post-emergence, or together with the seed of a crop plant. It is also possible to apply the herbicidal compositions or active compounds by applying seed, pretreated with the herbicidal compositions or active compounds, of a crop plant. If the active compounds are less well tolerated by certain crop plants, application techniques may be used in which the herbicidal compositions are sprayed, with the aid of the spraying equipment, in such a way that as far as possible they do not come into contact with the leaves of the sensitive crop plants, while the active compounds reach the leaves of undesirable plants growing underneath, or the bare soil surface (post-directed, lay-by).

In a further embodiment, the compounds of the formula I or the herbicidal compositions can be applied by treating seed.

The treatment of seed comprises essentially all procedures familiar to the person skilled in the art (seed dressing, seed coating, seed dusting, seed soaking, seed film coating, seed multilayer coating, seed encrusting, seed dripping and seed pelleting) based on the compounds of the formula I according to the invention or the compositions prepared therefrom. Here, the herbicidal compositions can be applied diluted or undiluted.

The term seed comprises seed of all types, such as, for example, corns, seeds, fruits, tubers, cuttings and similar forms. Here, preferably, the term seed describes corns and seeds.

The seed used can be seed of the useful plants mentioned above, but also the seed of transgenic plants or plants obtained by customary breeding methods.

The rates of application of active compound are from 0.001 to 3.0, preferably 0.01 to 1.0, kg/ha of active substance (a.s.), depending on the control target, the season, the target plants and the growth stage. To treat the seed, the compounds I are generally employed in amounts of from 0.001 to 10 kg per 100 kg of seed.

It may also be advantageous to use the compounds of the formula I in combination with safeners. Safeners are chemical compounds which prevent or reduce damage to useful plants without substantially affecting the herbicidal action of the compounds of the formula I on unwanted plants. They can be used both before sowing (for example in the treatment of seed, or on cuttings or seedlings) and before or after the emergence of the useful plant. The safeners and the compounds of the formula I can be used simultaneously or in succession. Suitable safeners are, for example, (quinolin-8-oxy)acetic acids, 1-phenyl-5-haloalkyl-1H-1,2,4-triazole-3-carboxylic acids, 1-phenyl-4,5-dihydro-5-alkyl-1H-pyrazole-3,5-dicarboxylic acids, 4,5-dihydro-5,5-diaryl-3-isoxazolecarboxylic acids, dichloroacetamides, alpha-oximinophenylacetonitriles, acetophenone oximes, 4,6-dihalo-2-phenylpyrimidines, N-[[4-(aminocarbonyl)phenyl]sulfonyl]-2-benzamides, 1,8-naphthalic anhydride, 2-halo-4-(haloalkyl)-5-thiazolecarboxylic acids, phosphorothiolates and O-phenyl N-alkylcarbamates and their agriculturally useful salts and, provided that they have an acid function, their agriculturally useful derivatives, such as amides, esters and thioesters.

To broaden the activity spectrum and to obtain synergistic effects, the compounds of the formula I can be mixed and jointly applied with numerous representatives of other herbicidal or growth-regulating groups of active compounds or with safeners. Suitable mixing partners are, for example, 1,2,4-thiadiazoles, 1,3,4-thiadiazoles, amides, aminophosphoric acid and its derivatives, aminotriazoles, anilides, aryloxy/heteroaryl-oxyalkanoic acids and their derivatives, benzoic acid and its derivatives, benzothiadiazinones, 2-(hetaroyl/aroyl)-1,3-cyclohexanediones, heteroaryl aryl ketones, benzylisoxazolidinones, meta-CF₃-phenyl derivatives, carbamates, quinoline carboxylic acid and its derivatives, chloroacetanilides, cyclohexenone oxime ether derivates, diazines, dichloropropionic acid and its derivatives, dihydrobenzofurans, dihydrofuran-3-ones, dinitroanilines, dinitrophenols, diphenyl ethers, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- and heteroaryloxyphenoxypropionic esters, phenylacetic acid and its derivatives, 2-phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides, uracils and also phenylpyrazolines and isoxazolines and their derivatives.

Moreover, it may be useful to apply the compounds I alone or in combination with other herbicides or else also mixed with further crop protection agents, jointly, for example with compositions for controlling pests or phytopathogenic fungi or bacteria. Also of interest is the miscibility with mineral salt solutions which are employed for alleviating nutritional and trace element deficiencies. Other additives such as nonphytotoxic oils and oil concentrates may also be added.

Examples of herbicides which can be used in combination with the piperazinedione compounds of the formula I according to the present invention are:

b1) from the group of the lipid biosynthesis inhibitors:

alloxydim, alloxydim-sodium, butroxydim, clethodim, clodinafop, clodinafop-propargyl, cycloxydim, cyhalofop, cyhalofop-butyl, diclofop, diclofop-methyl, fenoxaprop, fenoxaprop-ethyl, fenoxaprop-P, fenoxaprop-P-ethyl, fluazifop, fluazifop-butyl, fluazifop-P, fluazifop-P-butyl, haloxyfop, haloxyfop-methyl, haloxyfop-P, haloxyfop-P-methyl, metamifop, pinoxaden, profoxydim, propaquizafop, quizalofop, quizalofop-ethyl, quizalofop-tefuryl, quizalofop-P, quizalofop-P-ethyl, quizalofop-P-tefuryl, sethoxydim, tepraloxydim, tralkoxydim, benfuresate, butylate, cycloate, dalapon, dimepiperate, EPTC, esprocarb, ethofumesate, flupropanate, molinate, orbencarb, pebulate, prosulfocarb, TCA, thiobencarb, tiocarbazil, triallate and vernolate;

b2) from the group of the ALS inhibitors:

amidosulfuron, azimsulfuron, bensulfuron, bensulfuron-methyl, bispyribac, bispyribac-sodium, chlorimuron, chlorimuron-ethyl, chlorsulfuron, cinosulfuron, cloransulam, cloransulam-methyl, cyclosulfamuron, diclosulam, ethametsulfuron, ethametsulfuron-methyl, ethoxysulfuron, flazasulfuron, florasulam, flucarbazone, flucarbazone-sodium, flucetosulfuron, flumetsulam, flupyrsulfuron, flupyrsulfuron-methyl-sodium, foramsulfuron, halosulfuron, halosulfuron-methyl, imazamethabenz, imazamethabenz-methyl, imazamox, imazapic, imazapyr, imazaquin, imazethapyr, imazosulfuron, iodosulfuron, iodosulfuron-methyl-sodium, mesosulfuron, metosulam, metsulfuron, metsulfuron-methyl, nicosulfuron, orthosulfamuron, oxasulfuron, penoxsulam, primisulfuron, primisulfuron-methyl, propoxycarbazone, propoxycarbazone-sodium, prosulfuron, pyrazosulfuron, pyrazosulfuron-ethyl, pyribenzoxim, pyrimisulfan, pyriftalid, pyriminobac, pyriminobac-methyl, pyrithiobac, pyrithiobac-sodium, pyroxsulam, rimsulfuron, sulfometuron, sulfometuron-methyl, sulfosulfuron, thiencarbazone, thiencarbazone-methyl, thifensulfuron, thifensulfuron-methyl, triasulfuron, tribenuron, tribenuron-methyl, trifloxysulfuron, triflusulfuron, triflusulfuron-methyl and tritosulfuron;

b3) from the group of the photosynthesis inhibitors:

ametryn, amicarbazone, atrazine, bentazone, bentazone-sodium, bromacil, bromofenoxim, bromoxynil and its salts and esters, chlorobromuron, chloridazone, chlorotoluron, chloroxuron, cyanazine, desmedipham, desmetryn, dimefuron, dimethametryn, diquat, diquat-dibromide, diuron, fluometuron, hexazinone, ioxynil and its salts and esters, isoproturon, isouron, karbutilate, lenacil, linuron, metamitron, methabenzthiazuron, metobenzuron, metoxuron, metribuzin, monolinuron, neburon, paraquat, paraquat-dichloride, paraquat-dimetilsulfate, pentanochlor, phenmedipham, phenmedipham-ethyl, prometon, prometryn, propanil, propazine, pyridafol, pyridate, siduron, simazine, sinietryn, tebuthiuron, terbacil, terbumeton, terbuthylazine, terbutryn, thidiazuron and trietazine;

b4) from the group of the protoporphyrinogen-IX oxidase inhibitors:

acifluorfen, acifluorfen-sodium, azafenidin, bencarbazone, benzfendizone, bifenox, butafenacil, carfentrazone, carfentrazone-ethyl, chlomethoxyfen, cinidon-ethyl, fluazolate, flufenpyr, flufenpyr-ethyl, flumiclorac, flumiclorac-pentyl, flumioxazin, fluoroglycofen, fluoroglycofen-ethyl, fluthiacet, fluthiacet-methyl, fomesafen, halosafen, lactofen, oxadiargyl, oxadiazon, oxyfluorfen, pentoxazone, profluazol, pyraclonil, pyraflufen, pyraflufen-ethyl, saflufenacil, sulfentrazone, thidiazimin, 2-chloro-5-[3,6-dihydro-3-methyl-2,6-dioxo-4-(trifluoromethyl)-[(2H)-pyrimidinyl]-4-fluoro-N-[(isopropyl)-methylsulfamoyl]benzamide (CAS 372137-35-4), ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6), N-ethyl-3-(2,6-dichloro-4-trifluoromethylphenoxy)-5-methyl-1H-pyrazole-1-carboxamide (CAS 452098-92-9), N-tetrahydrofurfuryl-3-(2,6-dichloro-4-trifluoromethylphenoxy)-5-methyl-1H-pyrazole-1-carboxamide (CAS 915396-43-9), N-ethyl-3-(2-chloro-6-fluoro-4-trifluoromethylphenoxy)-5-methyl-1H-pyrazole-1-carboxamide (CAS 452099-05-7) and N-tetrahydrofurfuryl-3-(2-chloro-6-fluoro-4-trifluoromethylphenoxy)-5-methyl-1H-pyrazole-1-carboxamide (CAS 45100-03-7);

b5) from the group of the bleacher herbicides:

aclonifen, amitrol, beflubutamid, benzobicyclon, benzofenap, clomazone, diflufenican, fluridone, fluorochloridone, flurtamone, isoxaflutole, mesotrione, norflurazon, picolinafen, pyrasulfutole, pyrazolynate, pyrazoxyfen, sulcotrione, tefuryltrione, tembotrione, topramezone, 4-hydroxy-3-[[2-[(2-methoxyethoxy)methyl]-6-(trifluoromethyl)-3-pyridyl]carbonyl]bicyclo[3.2.1]oct-3-en-2-one (CAS 352010-68-5) and 4-(3-trffluoromethylphenoxy)-2-(4-trifluoromethylphenyl)pyrimidine (CAS 180608-33-7);

b6) from the group of the EPSP synthase inhibitors:

glyphosate, glyphosate-isopropylammonium and glyphosate-trimesium (sulfosate);

b7) from the group of the glutamine synthase inhibitors:

bilanaphos (bialaphos), bilanaphos-sodium, glufosinate and glufosinate-ammonium;

b8) from the group of the DHP synthase inhibitors:

asulam;

b9) from the group of the mitose inhibitors:

amiprophos, amiprophos-methyl, benfluralin, butamiphos, butralin, carbetamide, chlorpropham, chlorthal, chlorthal-dimethyl, dinitramine, dithiopyr, ethalfluralin, fluchloralin, oryzalin, pendimethalin, prodiamine, propham, propyzamide, tebutam, thiazopyr and trifluralin;

b10) from the group of the VLCFA inhibitors:

acetochlor, alachlor, anilofos, butachior, cafenstrole, dimethachlor, dimethanamid, dimethenamid-P, diphenamid, fentrazamide, flufenacet, mefenacet, metazachlor, metolachlor, metolachlor-S, naproanilide, napropamide, pethoxamid, piperophos, pretilachlor, propachlor, propisochlor, pyroxasulfone (KIH-485) and thenyichlor;

Compounds of the formula 2:

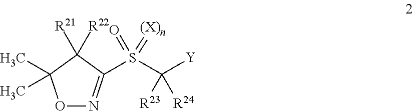

in which the variables have the following meanings:
Y is phenyl or 5- or 6-membered heteroaryl as defined at the outset, which radicals may be substituted by one to three groups R^{aa};
R21, R22, R²³, R²⁴ are H, halogen or C₁-C₄-alkyl;
X is O or NH;
n is 0 or 1.

Compounds of the formula 2 have in particular the following meanings:

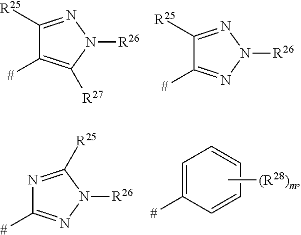

### Y is

where # denotes the bond to the skeleton of the molecule;
R²¹, R²², R²³, R²⁴ are H, Cl, F or CH₃;
R²⁵ is halogen, C₁-C₄-alkyl or C₁-C₄-haloalkyl;
R²⁶ is C₁-C₄-alkyl;
R²⁷ is halogen, C₁-C₄-alkoxy or C₁-C₄-haloalkoxy;
R²⁸ is H, halogen, C₁-C₄-alkyl, C₁-C₄-haloalkyl or C₁-C₄-haloalkoxy;
m is 0, 1, 2 or 3;
X is oxygen;
n is 0 or 1.

Preferred compounds of the formula 2 have the following meanings:

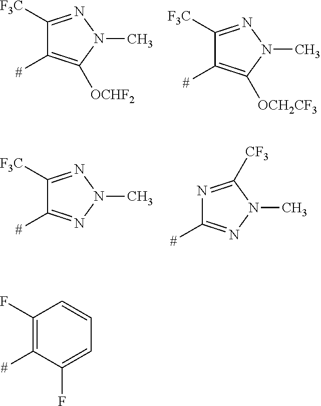

### Y is

R²¹ is H;
R²², R²³ are F;
R²⁴ is H or F;
X is oxygen;
n is 0 or 1.

Particularly preferred compounds of the formula 2 are:
3-[5-(2,2-difluoroethoxy)-1-methyl-3-trifluoromethyl-1H-pyrazol-4-ylmethanesulfonyl]-4-fluoro-5,5-dimethyl-4,5-dihydroisoxazole; 3-{[5-(2,2-difluoroethoxy)-1-methyl-3-trifluoromethyl-1H-pyrazol-4-yl]fluoromethanesulfonyl}-5,5-dimethyl-4,5-dihydroisoxazole; 4-(4-fluoro-5,5-dimethyl-4,5-dihydroisoxazole-3-sulfonylmethyl)-2-methyl-5-trifluoromethyl-2H-[1,2,3]triazole; 4-[(5,5-dimethyl-4,5-dihydroisoxazole-3-sulfonyl)fluoromethyl]-2-methyl-5-trifluoromethyl-2H-[1,2,3]triazole; 4-(5,5-dimethyl-4,5-dihydroisoxazole-3-sulfonylmethyl)-2-methyl-5-trifluoromethyl-2H-[1,2,3]triazole; 3-{[5-(2,2-difluoroethoxy)-1-methyl-3-trifluoromethyl-1H-pyrazol-4-yl]difluoromethanesulfonyl}-5,5-dimethyl-4,5-dihydroisoxazole; 4-[(5,5-dimethyl-4,5-dihydroisoxazole-3-sulfonyl)difluoromethyl]-2-methyl-5-trifluoromethyl-2H-[1,2,3]-triazole; 3{[5-(2,2-difluoroethoxy)-1-methyl-3-trifluoromethyl-1H-pyrazol-4-yl]difluoromethanesulfonyl}-4-fluoro-5,5-dimethyl-4,5-dihydroisoxazole; 4-[difluoro-(4-fluoro-5,5-dimethyl-4,5-dihydroisoxazole-3-sulfonyl)-methyl]-2-methyl-5-trifluoromethyl-2H-[1,2,3]triazole;

b11) from the group of the cellulose biosynthesis inhibitors:

chlorthiamid, dichlobenil, flupoxam and isoxaben;

b12) from the group of the decoupler herbicides:

dinoseb, dinoterb and DNOC and its salts;

b13) from the group of the auxin herbicides:

2,4-D and its salts and esters, 2,4-DB and its salts and esters, aminopyralid and its salts such as aminopyralid-tris(2-hydroxypropyl)ammonium and its esters, benazolin, benazolin-ethyl, chloramben and its salts and esters, clomeprop, clopyralid and its salts and esters, dicamba and its salts and esters, dichlorpropand its salts and esters, dichlorprop-P and its salts and esters, fluoroxypyr, fluoroxypyr-butomethyl, fluoroxypyr-meptyl, MCPA and its salts and esters, MCPA-thioethyl, MCPB and its salts and esters, mecopropand its salts and esters, mecoprop-P and its salts and esters, picloram and its salts and esters, quinclorac, quinmerac, TBA (2,3,6) and its salts and esters, triclopyr and its salts and esters, and 5,6-dichloro-2-cyclopropyl-4-pyrimidinecarboxylic acid (CAS 858956-08-8) and its salts and esters;

b14) from the group of the auxin transport inhibitors: diflufenzopyr, diflufenzopyr-sodium, naptalam and naptalam-sodium;

b15) from the group of the other herbicides: bromobutide, chlorflurenol, chlorflurenol-methyl, cinmethylin, cumyluron, dalapon, dazomet, difenzoquat, difenzoquat-metilsulfate, dimethipin, DSMA, dymron, endothal and its salts, etobenzanid, flamprop, flamprop-isopropyl, flamprop-methyl, flamprop-M-isopropyl, flamprop-M-methyl, flurenol, flurenol-butyl, flurprimidol, fosamine, fosamine-ammonium, indanofan, maleic hydrazide, mefluidide, metam, methyl azide, methyl bromide, methyl-dymron, methyl iodide, MSMA, oleic acid, oxaziclomefone, pelargonic acid, pyributicarb, quinoclamine, triaziflam, tridiphane and 6-chloro-3-(2-cyclopropyl-6-methylphenoxy)-4-pyridazinol (CAS 499223-49-3) and its salts and esters.

Examples of preferred safeners are benoxacor, cloquintocet, cyometrinil, cyprosulfamide, dichlormid, dicyclonone, dietholate, fenchlorazole, fenclorim, flurazole, fluxofenim, furilazole, isoxadifen, mefenpyr, mephenate, naphthalic anhydride, oxabetrinil, 4-(dichloroacetyl)-1-oxa-4-azaspiro[4.5]decane (MON4660, CAS 71526-07-3) and 2,2,5-trimethyl-3-(dichloroacetyl)-1,3-oxazolidine (R-29148, CAS 52836-31-4). The active compounds of groups b1) to b15) and the safeners are known herbicides and safeners, see, for example, The Compendium of Pesticide Common Names (http://www.alanwood.net/pesticides/); B. Hock, C. Fedtke, R. R. Schmidt, Herbizide [Herbicides], Georg Thieme Verlag, Stuttgart, 1995. Further herbicidally active compounds are known from WO 96/26202, WO 97/41116, WO 97/41117, WO 97/41118, WO 01/83459 and WO 2008/074991 and from W. Kramer et al. (ed.) “Modern Crop Protection Compounds”, Vol. 1, Wiley VCH, 2007 and the literature quoted therein.

The compounds I and the compositions according to the invention may also have a plant-strengthening action. Accordingly, they are suitable for mobilizing the defense system of the plants against attack by unwanted microorganisms, such as harmful fungi, but also viruses and bacteria. Plant-strengthening (resistance-inducing) substances are to be understood as meaning, in the present context, those substances which are capable of stimulating the defense system of treated plants in such a way that, when subsequently inoculated by unwanted microorganisms, the treated plants display a substantial degree of resistance to these microorganisms.

The compounds I can be employed for protecting plants against attack by unwanted microorganisms within a certain period of time after the treatment. The period of time within which their protection is effected generally extends from 1 to 28 days, preferably from 1 to 14 days, after the treatment of the plants with the compounds I, or, after treatment of the seed, for up to 9 months after sowing.

The compounds I and the compositions according to the invention are also suitable for increasing the harvest yield.

Moreover, they have reduced toxicity and are tolerated well by the plants. Hereinbelow, the preparation of piperazine compounds of the formula I is illustrated by way of examples, without limiting the subject matter of the present invention to the examples shown.

### SYNTHESIS EXAMPLES

With appropriate modification of the starting materials, the procedures given in the synthesis examples below were used to obtain further compounds I. The compounds obtained in this manner are listed in the table that follows, together with physical data.

The products shown below were characterized by determination of the melting point, by NMR spectroscopy or by the masses ([m/z]) or retention time (RT; [min.]) determined by HPLC-MS spectrometry.

[HPLC-MS=high performance liquid chromatography coupled with mass spectrometry; HPLC column:
a) RP-18 column (Chromolith Speed ROD from Merck KgaA, Germany), 50*4.6 mm; mobile phase: acetonitrile+0.1% trifluoroacetic acid (TFA)/water+0.1% TFA, using a gradient of from 5:95 to 100:0 over 5 minutes at 40° C., flow rate 1.8 ml/min; or
b) RP-18 column (XTerra MS 5 mm from Waters), mobile phase: acetonitrile+0.1% formic acid (A)/water+0.1% formic acid (B) using a gradient from 5:95 (NB) to 100:0 (NB) over 8 minutes at 20-25° C., flow rate 2 ml/min.
   MS: Quadrupole electrospray ionization, 80 V (positive mode).]

Unless indicated otherwise, the HPLC/MS data were obtained using method a).

### I. Preparation Examples

### EXAMPLE 1

### Preparation of 2-benzyl-5-[1-(2-cyanophenyl)meth-(Z)-ylidene]-2,4-dimethyl-3,6-dioxopiperazine-1-carbonitrile

At 0° C., 88 mg of NaH (60% strength in paraffin oil) were added a little at a time to a solution of 690 mg of 2-[5-benzyl-1,5-dimethyl-3,6-dioxopiperazin-(22)-ylidenemethyl]-benzonitrile [cf. PCT/EP2008/057329] in 5 ml of dimethylformamide (DMF). After one hour of stirring at 0° C., a solution of 263.7 mg of bromoacetonitrile in 2 ml of DMF was added slowly. After a further two hours of stirring at 0° C. and 18 hours of stirring at 20-25° C., 5% strength aqueous citric acid was added to the reaction mixture, and the reaction mixture was extracted with CH₂Cl₂. The combined organic phases were, after drying, freed from the solvent. The residue gave, after recrystallization from ethyl acetate, 373 mg (49% of theory) of the title compound.

### EXAMPLE 2

### Preparation of 3-[5-benzyl-4-methoxymethyl-1,5-dimethyl-3,6-dioxopiperazin-(2Z-ylidenemethyl]pyridine-2-carbonitrile

At 0° C., 88 mg of NaH (60% strength in paraffin oil) were added a little at a time to a solution of 692 mg of 3-[5-benzyl-1,5-dimethyl-3,6-dioxopiperazin-(2Z)-ylidenemethyl]-pyridine-2-carbonitrile [cf. PCT/EP2008/057329] in 5 ml of DMF. After one hour of stirring at 0° C., a solution of 274.9 mg of bromomethyl methyl ether in 2 ml of DMF was added slowly. After a further two hours of stirring at 0° C. and 18 hours of stirring at 20-25° C., 5% strength aqueous citric acid was added to the reaction mixture and the reaction mixture was extracted with CH₂Cl₂. The combined organic phases were, after drying, freed from the solvent. The residue gave, after chromatography on silica gel (cyclohexane/ethyl acetate), 281 mg (36% of theory) of the title compound.

### EXAMPLE 3

### Preparation of 3-[4-acetyl-5-benzyl-1,5-dimethyl-3,6-dioxopiperazin-(2Z)-ylidenemethyl]pyridine-2-carbonitrile

At 0° C., 88 mg of NaH (60% strength in paraffin oil) were added a little at a time to a solution of 692 mg (2 mmol) of 3-[5-benzyl-1,5-dimethyl-3,6-dioxopiperazin-(2Z)-ylidenemethyl]pyridine-2-carbonitrile [cf. PCT/EP2008/057329] in 6 ml of DMF. After two hours of stirring at 0° C., a solution of 172.7 mg of acetyl chloride in 1.5 ml of DMF was slowly added dropwise. After a further hour of stirring at 0° C. and 18 hours of stirring at 20-25° C., the reaction mixture was stirred into a 5% strength citric acid solution, and the precipitated product was filtered off and dried. This gave, after recrystallization from ethyl acetate, 258 mg (33% of theory) of the title compound.

### EXAMPLE 4

### Preparation of 2-[5-benzyl-4-methanesulfonyl-1,5-dimethyl-3,6-dioxopiperazin-(2Z)-ylidenemethyl]benzonitrile

At 0° C., 88 mg of NaH (60% strength in paraffin oil) were added a little at a time to a solution of 690 mg of 2-[5-benzyl-1,5-dimethyl-3,6-dioxopiperazin-(2Z)-ylidenemethyl]-benzonitrile [cf. PCT/EP2008/057329] in 5 ml of DMF. After one hour of stirring at 0° C., a solution of 251.9 mg of methanesulfonyl chloride in 2 ml of DMF was added slowly. After further stirring of the reaction mixture at 0° C. and 18 hours of stirring at 20-25° C., 50 ml of 5% strength citric acid were added and the reaction mixture was extracted with CH₂Cl₂. The combined organic phases were, after drying, freed from the solvent. The residue gave, after chromatography on silica gel (cyclohexane/ethyl acetate), 323 mg (38% of theory) of the title compound.

### EXAMPLE 5

### Preparation of 4-amino-3-benzyl-1,3-dimethyl-6-[1-(2-nitrophenyl)meth-(Z)-ylidene]piperazine-2,5-dione

At 0° C., 44 mg of NaH (60% strength in paraffin) were added to a solution of 365.4 mg of 3-benzyl-1,3-dimethyl-6-[1-(2-nitrophenyl)meth-(Z)-ylidene]piperazine-2,5-dione) [cf. PCT/EP2008/057329] in 5 ml of DMF, and the mixture was then stirred at 0° C. for one hour. After addition, a little at a time, of 218 mg of p-nitrobenzoyloxyamine, the mixture was stirred at 0° C. for one hour and then at 20-25° C. for two hours. Dilute citric acid was added to the reaction mixture, and the reaction mixture was extracted with CH₂Cl₂. The combined organic phases were, after drying, freed from the solvent. The residue gave, after chromatography on silica gel (cyclohexane/ethyl acetate) and preparative HPLC (acetonitrile/water+0.05% TFA), 28 mg (7% of theory) of the title compound as a mixture with the starting material (about 70% title compound).

TABLE I
Compounds of the formula I which correspond to the formula I.A' :

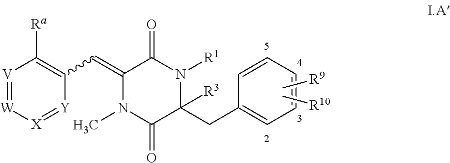

| | | | | | | | | | Iso- | phys. data (m.p.[° C.]; |
|---|---|---|---|---|---|---|---|---|---|---|
| No. | R^{a} | V | W | X | Y | R¹ | R³ | R⁹/R¹⁰ | mer^{*)} | HPLC: RT[min]/m/z) |
| I-1 | NO₂ | CH | CH | CH | CH | NH₂ | CH₃ | H | Z | 3.033/381.1 [M + H]⁺ |
| I-2 | CN | CH | CH | CH | CH | SO₂CH₃ | CH₃ | H | Z | 3.347/423.7 [M]⁺ |
| I-3 | NO₂ | CH | CH | CH | CH | SO₂CH₃ | CH₃ | H | Z | 3.60/444.15 [M + H]⁺ |
| I-4 | CN | CH | CH | CH | CH | CH₂CN | CH₃ | H | Z | 3.048/384.8 [M]⁺ |
| I-5 | CN | CH | CH | CH | CH | CH₂CO₂C₂H₅ | CH₃ | H | Z | 3.356/432.1 [M + H]⁺ |
| I-6 | CN | CH | CH | CH | CH | CH₂F | CH₃ | H | Z | 3.600/377.8 [M]⁺ |
| I-7 | CN | CH | CH | CH | CH | CH₂—COCH₂Cl | CH₃ | H | Z | 3.755/422.0 [M]⁺ |
| I-8 | NO₂ | CH | CH | CH | CH | CH₂—COCH₂Cl | CH₃ | H | Z | 3.811/442.1 [M]⁺ |
| I-9 | NO₂ | CH | CH | CH | CH | CH₂CN | CH₃ | H | Z | 3.304/405.1 [M + H]⁺ |
| I-10 | NO₂ | CH | CH | CH | CH | CH₂CO₂C₂H₅ | CH₃ | H | Z | 3.446/451.9 [M + H]⁺ |
| I-11 | NO₂ | CH | CH | CH | CH | CH₂F | CH₃ | H | Z | 3.859/398.1 [M + H]⁺ |
| I-12 | CN | CH | CH | CH | CH | CH₂C≡CCH₃ | CH₃ | H | Z | 3.065/398.3 [M + H]⁺ |
| I-13 | NO₂ | CH | CH | CH | CH | CH₂C≡CCH₃ | CH₃ | H | Z | 3.367/419.8 [M + H]⁺ |
| I-14 | CN | CH | CH | CH | CH | CH₂—C(CH₃)═CH₂ | CH₃ | H | Z | 3.365/400.8 [M + H]⁺ |
| I-15 | NO₂ | CH | CH | C—F | CH | CH₂—C(CH₃)═CH₂ | CH₃ | H | Z | 3.511/420.3 [M + H]⁺ |
| I-16 | CN | CH | CH | CH | CH | CH₂—CH═CHCH₃ | CH₃ | H | Z | 3.558/400.4 [M + H]⁺ |
| I-17 | NO₂ | CH | CH | CH | CH | CH₂—CH═CHCH₃ | CH₃ | H | Z | 3.609/419.5 [M + H]⁺ |
| I-18 | NO₂ | CH | CH | CH | CH | CH₂CH₂—CH═CH₂ | CH₃ | H | Z | 3.479/420.8 [M + H]⁺ |
| I-19 | CN | CH | CH | CH | CH | CH₂CH₂—CH═CH₂ | CH₃ | H | Z | 3.447/400.8 [M + H]⁺ |
| I-20 | NO₂ | CH | CH | CH | CH | (CH₂)₄CH₃ | CH₃ | H | Z | 4.123/436.8 [M + H]⁺ |
| I-21 | CN | CH | CH | CH | CH | (CH₂)₄CH₃ | CH₃ | H | Z | 4.073/415.8 [M]⁺ |
| I-22 | NO₂ | CH | CH | CH | CH | (CH₂)₅CH₃ | CH₃ | H | Z | 4.337/450.8 [M + H]⁺ |
| I-23 | CN | CH | CH | CH | CH | (CH₂)₅CH₃ | CH₃ | H | Z | 4.290/429.8 [M]⁺ |
| I-24 | NO₂ | CH | CH | CH | CH | CH₂-c-C₃H₅ | CH₃ | H | Z | 3.831/420.2 [M + H]⁺ |
| I-25 | CN | CH | CH | CH | CH | CH₂-c-C₃H₅ | CH₃ | H | Z | 3.775/400.4 [M + H]⁺ |
| I-26 | CN | CH | CH | CH | CH | (CH₂)₃CN | CH₃ | H | Z | 3.235/412.8 [M]⁺ |
| I-27 | NO₂ | CH | CH | CH | CH | CH₂C₆H₅ | CH₃ | H | Z | 3.831/456.2 [M + H]⁺ |
| I-28 | CN | CH | CH | CH | CH | CH₂C₆H₅ | CH₃ | H | Z | 3.845/436.4 [M + H]⁺ |
| I-29 | NO₂ | CH | CH | CH | CH | CH₂—(4-OCH₃—C₆H₄) | CH₃ | H | Z | 3.859/486.2 [M + H]⁺ |
| I-30 | CN | CH | CH | CH | CH | CH₂—(4-OCH₃—C₆H₄) | CH₃ | H | Z | 3.821/466.4 [M + H]⁺ |
| I-31 | CN | CH | CH | CH | CH | COC₆H₅ | CH₃ | H | Z | 4.009/472.2 [M + Na]⁺ |
| I-32 | CN | CH | CH | CH | CH | CH₂—(4-COOCH₃—C₆H₄) | CH₃ | H | Z | 3.727/494.7 [M + H]⁺ |
| I-33 | NO₂ | CH | CH | CH | CH | CH₂—CH═CHCl | CH₃ | H | Z | 3.744/441.7 [M + H]⁺ |
| I-34 | CN | CH | CH | CH | CH | CH₂—CH═CHCl | CH₃ | H | Z | 4.213/420.7 [M + H]⁺ |
| I-35 | NO₂ | CH | CH | CH | CH | CH₂—(4-NO₂—C₆H₄) | CH₃ | H | Z | 3.785/501.7 [M + H]⁺ |
| I-36 | CN | CH | CH | CH | CH | CH₂—(4-NO₂—C₆H₄) | CH₃ | H | Z | 3.739/481.7 [M + H]⁺ |
| I-37 | NO₂ | CH | CH | CH | CH | COC₆H₅ | CH₃ | H | Z | 3.995/470.7 [M + Na]⁺ |
| I-38 | NO₂ | CH | CH | CH | CH | (CH₂)₃CN | CH₃ | H | Z | 3.754/433.3 [M + H]⁺ |
| I-39 | NO₂ | CH | CH | CH | CH | (CH₂)₂CN | CH₃ | H | Z | 3.388/419.2 [M + H]⁺ |
| I-40 | CN | CH | CH | CH | CH | (CH₂)₂CN | CH₃ | H | Z | 3.231/398.9 [M]⁺ |
| I-41 | CN | N | CH | CH | CH | CH₂CN | CH₃ | H | Z | 2.887/386.2 [M + H]⁺ |
| I-42 | CN | N | CH | CH | CH | CH₂OCH₃ | CH₃ | H | Z | 2.918/391.2 [M + H]⁺ |
| I-43 | CN | N | CH | CH | CH | SO₂CH₃ | CH₃ | H | Z | 3.122/426.2 [M + H]⁺ |
| I-44 | CN | CH | CH | CH | CH | CH₂C═C(CH₃)₂ | CH₃ | H | Z | 3.774/414.8 [M + H]⁺ |
| I-45 | CN | N | CH | CH | CH | CH₂—(4-CO₂CH₃—C₆H₄) | CH₃ | H | Z | 3.497/495.2 [M + H]⁺ |
| I-46 | CN | N | CH | CH | CH | (CH₂)₃CN | CH₃ | H | Z | 2.924/417.5 [M + H]⁺ |
| I-47 | CN | N | CH | CH | CH | CH₂—(4-NO₂—C₆H₄) | CH₃ | H | Z | 3.518/482.2 [M + H]⁺ |
| I-48 | CN | N | CH | CH | CH | CH₂C═C(CH₃)₂ | CH₃ | H | Z | 3.533/414.8 [M + H]⁺ |
| I-49 | OCH₂CH₃ | N | CH | CH | CH | CH₂CN | CH₃ | H | Z | 3.289/405.3 [M + H]⁺ |
| I-50 | OCH₂CH₃ | N | CH | CH | CH | CH₂-c-C₃H₅ | CH₃ | H | Z | 3.726/420.4 [M + H]⁺ |
| I-51 | CF₃ | CH | CH | CH | CH | (CH₂)₅CH₃ | CH₃ | H | Z | 4.481/473.8 [M + H]⁺ |
| I-52 | CN | N | CH | CH | CH | CH₂—(4-OCH₃—C₆H₄) | CH₃ | H | E | 3.490/467.8 [M + H]⁺ |
| I-53 | CF₃ | N | CH | CH | CH | CH₂OCH₃ | CH₃ | H | Z | 132 |
| I-54 | CF₃ | N | CH | CH | CH | CH₂CN | CH₃ | H | Z | 185 |
| I-55 | CN | N | CH | CH | CH | CH₂—C(CO₂C₂H₅)═CH₂ | CH₃ | H | Z | 3.365/459.4 [M + H]⁺ |
| I-56 | CN | N | CH | CH | CH | CH₂—CH═CHCl | CH₃ | H | Z | 3.490/421.3 [M + H]⁺ |
| I-57 | OCF₃ | CH | CH | CH | CH | CH₂CN | CH₃ | H | Z | 3.632/444.9 [M + H]⁺ |
| I-58 | CF₃ | CH | CH | CH | CH | (CH₂)₄CH₃ | CH₃ | H | Z | 4.290/459.8 [M + H]⁺ |
| I-59 | CF₃ | CH | CH | CH | CH | CH₂CO₂C₂H₅ | CH₃ | H | Z | 3.752/475.7 [M + H]⁺ |
| I-60 | CF₃ | CH | CH | CH | CH | CH₂—CH═CHCH₃ | CH₃ | H | Z | 3.973/443.8 [M + H]⁺ |
| I-61 | CF₃ | CH | CH | CH | CH | CH₂C═₂C(CH₃)₂ | CH₃ | H | Z | 4.138/475.8 [M + H]⁺ |
| I-62 | CF₃ | CH | CH | CH | CH | CH₂C≡CCH₃ | CH₃ | H | Z | 3.842/441.7 [M + H]⁺ |
| I-63 | CF₃ | CH | CH | CH | CH | CH₂-c-C₃H₅ | CH₃ | H | Z | 3.957/443.8 [M + H]⁺ |
| I-64 | OCF₃ | CH | CH | CH | CH | (CH₂)₅CH₃ | CH₃ | H | Z | 4.544/489.8 [M + H]⁺ |
| I-65 | OCF₃ | CH | CH | CH | CH | (CH₂)₄CH₃ | CH₃ | H | Z | 4.356/475.8 [M + H]⁺ |
| I-66 | OCF₃ | CH | CH | CH | CH | CH₂CO₂C₂H₅ | CH₃ | H | Z | 3.822/491.7 [M + H]⁺ |
| I-67 | OCF₃ | CH | CH | CH | CH | CH₂—CH═CHCH₃ | CH₃ | H | Z | 4.042/459.7 [M + H]⁺ |
| I-68 | OCF₃ | CH | CH | CH | CH | CH₂C═C(CH₃)₂ | CH₃ | H | Z | 4.203/473.3 [M + H]⁺ |
| I-69 | OCF₃ | CH | CH | CH | CH | CH₂C≡CCH₃ | CH₃ | H | Z | 3.916/456.7 [M + H]⁺ |
| I-70 | OCF₃ | CH | CH | CH | CH | CH₂-c-C₃H₅ | CH₃ | H | Z | 4.031/459.7 [M + H]⁺ |
| I-71 | OCHF₂ | CH | CH | CH | CH | CH₂C═C(CH₃)₂ | CH₃ | H | Z | 3.739/455.4 [M + H]⁺ |
| I-72 | OCH₂CH₃ | N | CH | CH | CH | CH₂OCH₃ | CH₃ | H | Z | 113 |
| I-73 | CN | N | CH | CH | CH | CH₂CH₂—C(CH₃)═CH₂ | CH₃ | H | Z | 3.572/415.4 [M + H]⁺ |
| I-74 | OCHF₂ | CH | CH | CH | CH | (CH₂)₅CH₃ | CH₃ | H | Z | 4.364/471.4 [M + H]⁺ |
| I-75 | OCHF₂ | CH | CH | CH | CH | (CH₂)₄CH₃ | CH₃ | H | Z | 4.167/457.4 [M + H]⁺ |
| I-76 | OCHF₂ | CH | CH | CH | CH | CH₂CO₂C₂H₅ | CH₃ | H | Z | 3.922/473.3 [M + H]⁺ |
| I-77 | OCHF₂ | CH | CH | CH | CH | CH₂—CH═CHCH₃ | CH₃ | H | Z | 3.922/473.3 [M + H]⁺ |
| I-78 | OCHF₂ | CH | CH | CH | CH | CH₂C≡CCH₃ | CH₃ | H | Z | 3.739/439.4 [M + H]⁺ |
| I-79 | OCHF₂ | CH | CH | CH | CH | CH₂-c-C₃H₅ | CH₃ | H | Z | 3.841/441.4 [M + H]⁺ |
| I-80 | OCH₂CH₃ | N | CH | CH | CH | CH₂CO₂C₂H₅ | CH₃ | H | Z | 3.51/452.4 [M + H]⁺ |
| I-81 | Br | N | CH | CH | CH | CH₂CO₂C₂H₅ | CH₃ | H | Z | 3.269/488.3 [M + H]⁺ |
| I-82 | Br | N | CH | CH | CH | CH₂-c-C₃H₅ | CH₃ | H | Z | 3.497/456.2 [M + H]⁺ |
| I-83 | Br | N | CH | CH | CH | CH₂OCH₃ | CH₃ | H | Z | 3.142/445.1 [M + H]⁺ |
| I-84 | OCHF₂ | CH | CH | CH | CH | CH₂CN | CH₃ | H | Z | 3.448/426.9 [M + H]⁺ |
| I-85 | CF₃ | N | CH | CH | C—OCH₃ | CH₂OCH₃ | CH₃ | H | Z | 3.224/463.7 [M + H]⁺ |
| I-86 | CN | N | CH | CH | CH | CH₂SCH₃ | CH₃ | H | Z | 3.082/359.8 [M − SCH₃]⁺ |
| I-87 | CN | N | CH | CH | CH | (CH₂)₃OH | CH₃ | H | Z | 3.616/405.8 [M + H]⁺ |
| I-88 | CN | N | CH | CH | CH | CH₂—COC₆H₅ | CH₃ | H | Z | 3.239/464.3 [M]⁺ |
| I-89 | OCH₂CH₃ | N | CH | C—F | CH | CH₂OCH₃ | CH₃ | 3-F | Z | 3.534/445.7 [M]⁺ |
| I-90 | CN | N | CH | CH | CH | CH₂—(2-Cl—C₆H₄) | CH₃ | H | Z | 3.752/472.1 [M + H]⁺ |
| I-91 | (CH₂)₃CH₃ | N | CH | CH | CH | CH₂OCH₃ | CH₃ | H | Z | 2.521/421.5 [M]⁺ |
| I-92 | CN | CH | CH | CH | CH | CH₂CH₂OCH₃ | CH₃ | H | Z | 3.110/404.8 [M + H]⁺ |
| I-93 | CN | CH | CH | CH | CH | CH₂SCH₃ | CH₃ | H | Z | 3.353/358.8 [M − SCH₃]⁺ |
| I-94 | CN | CH | CH | CH | CH | (CH₂)₃Cl | CH₃ | H | Z | 3.418/421.7 [M]⁺ |
| I-95 | NO₂ | CH | CH | CH | CH | (CH₂)₃Cl | CH₃ | H | Z | 3.463/441.7 [M]⁺ |
| I-96 | Br | N | CH | CH | CH | CH₂CH₂OCH₃ | CH₃ | H | Z | 97 |
| I-97 | Br | N | CH | CH | CH | CH₂OC₂H₅ | CH₃ | H | Z | 115 |
| I-98 | OCH₂CH₃ | N | CH | CH | CH | CH₂CH₂OC₂H₅ | CH₃ | H | Z | 3.545/437.9 [M]⁺ |
| I-99 | OCH₂CH₃ | N | CH | CH | CH | CH₂OCH₂CH₂OCH₃ | CH₃ | H | Z | 3.369/454.2 [M + H]⁺ |
| I-100 | CN | N | CH | CH | CH | CH₂OCH₂CH₂OCH₃ | CH₃ | H | Z | 132 |
| I-101 | CN | N | CH | CH | CH | CH₂OC₂H₅ | CH₃ | H | Z | 130 |
| I-102 | OCH₂CH₃ | N | CH | CH | CH | CH₂OC₂H₅ | CH₃ | H | Z | 62 |
| I-103 | OCH₃ | N | CH | CH | CH | CH₂OCH₃ | CH₃ | H | Z | 152 |
| I-104 | CF₂CF₃ | N | CH | CH | CH | CH₂OCH₃ | CH₃ | H | Z | 3.681/484.1 [M + H]⁺ |
| I-105 | OCH₂CH₃ | N | CH | CH | CH | CH₂—CHO | CH₃ | H | Z | 3.071/408.2 [M + H]⁺ |
| I-106 | NHCH₃ | N | CH | CH | CH | CH₂OCH₃ | CH₃ | H | Z | 2.208/394.5 [M]⁺ |
| I-107 | CN | N | CH | CH | CH | CH₂OC₂H₅ | CH₃ | H | Z | 3.063/405.1 [M + H]⁺ |
| I-108 | COCH₃ | N | CH | CH | CH | CH₂OCH₃ | CH₃ | H | Z | 126 |
| I-109 | CN | N | CH | CH | CH | CH₂OCH(CH₃)₂ | CH₃ | H | E | 3.337/419.1 [M + H]⁺ |
| I-110 | OCH₂CH₃ | N | CH | CH | CH | CH₂CH═N—OCH₃ (E) | CH₃ | H | Z | 3.290/437.2 [M + H]⁺ |
| I-111 | OCH₂CH₃ | N | CH | CH | CH | CH₂CH(OH)CH₃ | CH₃ | H | Z | 130 |
| I-112 | OCH₂CH₃ | N | CH | CH | CH | CH₂—CH═CHCO₂CH₃ | CH₃ | H | Z | 3.393/464.8 [M + H]⁺ |
| I-113 | OCH₂CH₃ | N | CH | CH | CH | CH₂CH₂OH | CH₃ | H | Z | 2.765/410.1 [M + H]⁺ |
| I-114 | OCH₂CH₃ | N | CH | CH | CH | CH₂CH(OCH₃)₂ | CH₃ | H | Z | 3.270/454.2 [M + H]⁺ |
| I-115 | OCH₂CH₃ | N | CH | CH | CH | CH₂CH═N—OH | CH₃ | H | Z | 139 |
| I-116 | OCH₂CH₃ | N | CH | CH | CH | CH₂COCH₃ | CH₃ | H | Z | 152 |
| I-117 | OCH₂CH₃ | N | CH | CH | CH | CH₂CH(OH)CH₂OH | CH₃ | H | Z | 2.651/440.1 [M + H]⁺ |
| I-118 | OCH₂CH₃ | N | CH | CH | CH | CH₂CH(OH)CH₂Cl | CH₃ | H | Z | 3.091/458.1 [M + H]⁺ |
| I-119 | OCH₂CH₃ | N | CH | CH | CH | CH₂CH₂N(CH₃)₂ | CH₃ | H | Z | 135 |
| I-120 | OCH₂CH₃ | N | CH | CH | CH | CH₂CH═N—OCH₃ (Z) | CH₃ | H | Z | 3.310/437.2 [M + H]⁺ |
| I-121 | CN | CH | CH | CH | CH | CH₂OCH₃ | CH₃ | H | Z | 3.142/358.8 [M − OCH₃]⁺ |
| I-122 | CN | N | CH | CH | CH | CH₂OCOCH₃ | CH₃ | H | Z | 2.973/359.1 [M − OCOCH₃]⁺ |
| I-123 | Cl | N | CH | CH | CH | CH₂OCH₂CH₂Cl | CH₃ | H | Z | 150 |
| I-124 | Cl | N | CH | CH | CH | CH₂OCH(CH₃)₂ | CH₃ | H | Z | 139 |
| I-125 | CN | N | CH | CH | CH | CH₂OCH₂CH₂Cl | CH₃ | H | Z | 168 |
| I-126 | OCH₂CH₃ | N | CH | CH | CH | CH₂OCH₂CH₂Cl | CH₃ | H | Z | 3.530/458.1 [M + H]⁺ |
| I-127 | OCH₃ | N | CH | CH | CH | CH₂OCH₂CH₂Cl | CH₃ | H | Z | 3.331/444.1 [M + H]⁺ |
| I-128 | Cl | N | CH | CH | CH | CH₂OC₂H₅ | CH₃ | H | Z | 125 |
| I-129 | Cl | N | CH | CH | CH | CH₂OCH₂C₆H₅ | CH₃ | H | Z | 104 |
| I-130 | Cl | N | CH | CH | CH | CH₂OCOC(CH₃)₃ | CH₃ | H | Z | 170 |
| I-131 | Cl | N | CH | CH | CH | CH₂OCH₂CH₂Si(CH₃)₃ | CH₃ | H | Z | 4.259/486.1 [M]⁺ |
| I-132 | OCH₃ | N | CH | CH | CH | CH₂OC₂H₅ | CH₃ | H | Z | 132 |
| I-133 | OCH₃ | N | CH | CH | CH | CH₂OCH₂C₆H₅ | CH₃ | H | Z | 3.841/472.1 [M + H]⁺ |
| I-134 | OCH₂CH₃ | N | CH | CH | CH | CH₂OCH₂C₆H₅ | CH₃ | H | Z | 4.022/486.1 [M + H]⁺ |
| I-135 | OCH₃ | N | CH | CH | CH | CH₂OCH₂CH₂Si(CH₃)₃ | CH₃ | H | Z | 4.109/482.2 [M + H]⁺ |
| I-136 | OCH₂CH₃ | N | CH | CH | CH | CH₂OCH₂CH₂Si(CH₃)₃ | CH₃ | H | Z | 4.272/496.2 [M + H]⁺ |
| I-137 | CN | N | CH | CH | CH | CH₂OCH(CH₃)₂ | CH₃ | H | Z | 3.434/419.1 [M + H]⁺ |
| I-138 | COCH₃ | N | CH | CH | CH | CH₂OCH₂C₆H₅ | CH₃ | H | Z | 57 |
| I-139 | COCH₃ | N | CH | CH | CH | CH₂OCOC(CH₃)₃ | CH₃ | H | Z | 3.601/478.1 [M + H]⁺ |
| I-140 | COCH₃ | N | CH | CH | CH | CH₂OC₂H₅ | CH₃ | H | Z | 66 |
| I-141 | CN | N | CH | CH | CH | CH₂OH | CH₃ | H | Z | 2.412/377.1 [M + H]⁺ |
| I-142 | CN | N | CH | CH | CH | CH₂OCH₂CH₂Si(CH₃)₃ | CH₃ | H | Z | 4.109/477.1 [M + H]⁺ |
| I-143 | CN | CH | CH | CH | CH | CH₂OCH(CH₃)₂ | CH₃ | H | Z | 3.684/358.1 |
| | | | | | | | | | | [M − OCH(CH₃)₂]⁺ |
| I-144 | CN | CH | CH | CH | CH | CH₂OCH₂CH₂Si(CH₃)₃ | CH₃ | H | Z | 4.106/359.1 [M − OR]⁺ |
| I-145 | NO₂ | CH | CH | CH | CH | CH₂OCH₂CH₂Si(CH₃)₃ | CH₃ | H | Z | 4.332/379.0 [M − OR]⁺ |
| I-146 | CN | CH | CH | CH | CH | CH₂OCH₂CH₂Si(CH₃)₃ | CH₃ | H | E | 4.037/359.1 [M − OR]⁺ |
| I-147 | COCH₃ | N | CH | CH | CH | CH₂OCH₂CH₂Si(CH₃)₃ | CH₃ | H | Z | 3.831/494.2 [M + H]⁺ |
| I-148 | CN | CH | CH | CH | CH | CH₂CH₂CO₂CH₃ | CH₃ | H | Z | 3.218/432.1 [M + H]⁺ |
| I-149 | CN | N | CH | CH | CH | CH₂CO₂C₂H₅ | CH₃ | H | Z | 2.956/433.1 [M + H]⁺ |
| I-150 | OCH₂CH₃ | N | CH | CH | CH | CH₂OCH₂CH₂OC₂H₅ | CH₃ | H | Z | 3.549/468.2 [M + H]⁺ |
| I-151 | CN | N | CH | CH | CH | CH₂CH₂CO₂H | CH₃ | H | Z | 2.643/419.1 [M + H]⁺ |
| I-152 | CN | CH | CH | CH | CH | CH₂CH₂CO₂H | CH₃ | H | Z | 2.952/418.1 [M + H]⁺ |
| I-153 | NO₂ | CH | CH | CH | CH | CH₂CH₂CO₂CH₃ | CH₃ | H | Z | 3.263/452.1 [M + H]⁺ |
| I-154 | CN | N | CH | CH | CH | CH₂CO₂H | CH₃ | H | Z | 2.619/405.0 [M + H]⁺ |
| I-155 | CN | CH | CH | CH | CH | CH₂CO₂H | CH₃ | H | Z | 2.926/404.1 [M + H]⁺ |
| I-156 | CN | N | CH | CH | CH | CH₂CH₂OH | CH₃ | H | Z | 2.391/391.1 [M + H]⁺ |

^{*)}This refers to the stereochemistry of the double bond at the piperazine skeleton. The compounds prepared are in each case the
racemate.

TABLE II
Compounds of the formula I which correspond to the formula I.B′:

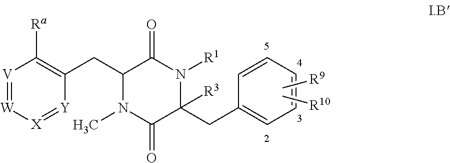

| | | | | | | | | | cis/ | phys. data (m.p.[° C.]; |
|---|---|---|---|---|---|---|---|---|---|---|
| No. | R^{a} | V | W | X | Y | R¹ | R³ | R⁹/R¹⁰ | trans^{*)} | HPLC: RT[min]/m/z) |
| II-1 | CN | CH | CH | CH | CH | CH₂CN | CH₃ | H | cis | 2.886/387.8 [M + H]⁺ |
| II-2 | CN | CH | CH | CH | CH | SO₂CH₃ | CH₃ | H | cis | 3.211/426.7 [M + H]⁺ |
| II-3 | CN | CH | CH | CH | CH | COC₆H₅ | CH₃ | H | cis | 3.662/452.7 [M + H]⁺ |
| II-4 | CN | CH | CH | CH | CH | CH₂C₆H₅ | CH₃ | H | cis | 2.712/438.8 [M + H]⁺ |
| II-5 | CN | CH | CH | CH | CH | CH₂C(CH₃)═CH₂ | CH₃ | H | cis | 2.468/402.2 [M + H]⁺ |
| II-6 | NO₂ | CH | CH | CH | CH | CH₂CN | CH₃ | H | cis | 3.000/407.7 [M + H]⁺ |
| II-7 | NO₂ | CH | CH | CH | CH | SO₂CH₃ | CH₃ | H | cis | 3.310/445.7 [M + H]⁺ |
| II-8 | NO₂ | CH | CH | CH | CH | COC₆H₅ | CH₃ | H | cis | 3.737/471.7 [M + H]⁺ |
| II-9 | NO₂ | CH | CH | CH | CH | CH₂C₆H₅ | CH₃ | H | cis | 3.537/458.8 [M + H]⁺ |
| II-10 | NO₂ | CH | CH | CH | CH | CH₂CH(CH₃)═CH₂ | CH₃ | H | cis | 3.317/422.8 [M + H]⁺ |
| II-11 | CN | CH | CH | CH | CH | (CH₂)₄CH₃ | CH₃ | H | cis | 4.076/418.8 [M + H]⁺ |
| II-12 | CN | CH | CH | CH | CH | CH₂CO₂CH₂CH₃ | CH₃ | H | cis | 3.043/434.8 [M + H]⁺ |
| II-13 | CN | CH | CH | CH | CH | CH₂CH═CHCH₃ | CH₃ | H | cis | 3.134/402.8 [M + H]⁺ |
| II-14 | CN | CH | CH | CH | CH | CH₂CH═C(CH₃)₂ | CH₃ | H | cis | 3.314/416.8 [M + H]⁺ |
| II-15 | CN | CH | CH | CH | CH | CH₂C≡CCH₃ | CH₃ | H | cis | 3.314/416.8 [M + H]⁺ |
| II-16 | CN | CH | CH | CH | CH | CH₂-c-C₃H₅ | CH₃ | H | cis | 3.658/402.8 [M + H]⁺ |
| II-17 | NO₂ | CH | CH | CH | CH | (CH₂)₄CH₃ | CH₃ | H | cis | 4.161/438.8 [M + H]⁺ |
| II-18 | NO₂ | CH | CH | CH | CH | CH₂CO₂CH₂CH₃ | CH₃ | H | cis | 3.22/454.8 [M + H]⁺ |
| II-19 | NO₂ | CH | CH | CH | CH | CH₂-c-C₃H5 | CH₃ | H | cis | 3.367/422.8 [M + H]⁺ |
| II-20 | NO₂ | CH | CH | CH | CH | CH₂CH═CHCH₃ | CH₃ | H | cis | 3.423/422.8 [M + H]⁺ |
| II-21 | NO₂ | CH | CH | CH | CH | CH₂CH═C(CH₃)₂ | CH₃ | H | cis | 3.286/436.8 [M + H]⁺ |
| II-22 | NO₂ | CH | CH | CH | CH | CH₂C≡CCH₃ | CH₃ | H | trans | 3.126/420.8 [M + H]⁺ |
| II-23 | NO₂ | CH | CH | CH | CH | (CH₂)₅CH₃ | CH₃ | H | cis | 3.494/402.8 [M + H]⁺ |
| II-24 | CN | CH | CH | CH | CH | (CH₂)₅CH₃ | CH₃ | H | cis | 4.292/432.8 [M + H]⁺ |
| II-25 | CN | CH | CH | CH | CH | CON(CH₃)₂ | CH₃ | H | cis | 3.737/471.7 [M + H]⁺ |
| II-26 | OCH₂CH₃ | N | CH | CH | CH | CH₂OCH₃ | CH₃ | H | cis | 115 |
| II-27 | CN | CH | CH | CH | CH | CH₂OCH₃ | CH₃ | H | cis | 2.885/360.8 [M − OMe]⁺ |
| II-28 | OCH₂CH₃ | N | CH | CH | CH | CH₂OCH₃ | CH₃ | H | trans | 104 |
| II-29 | OCH₃ | N | CH | CH | CH | CH₂OCH₃ | CH₃ | H | cis | 162 |
| II-30 | OCH₃ | N | CH | CH | CH | CH₂OCH₃ | CH₃ | H | trans | 2.682/398.3 [M + H]⁺ |
| II-31 | CH₂CF₃ | N | CH | CH | CH | CH₂OCH₃ | CH₃ | H | trans | 2.485/449.8 [M + H]⁺ |
| II-32 | OCH₂CH₃ | N | CH | CH | CH | CH₂OC₂H₅ | CH₃ | H | cis | 121 |
| II-33 | CF₂CF₃ | N | CH | CH | CH | CH₂OCH₃ | CH₃ | H | cis | 3.254/485.7 [M]⁺ |
| II-34 | CH₂CF₃ | N | CH | CH | CH | CH₂OCH₃ | CH₃ | H | cis | 2.486/449.8 [M]⁺ |
| II-35 | (CH₂)₃CH₃ | N | CH | CH | CH | CH₂OCH₃ | CH₃ | H | cis | 2.323/423.6 [M]⁺ |
| II-36 | (CH₂)₃CH₃ | N | CH | CH | CH | CH₂OCH₃ | CH₃ | H | trans | 2.510/423.6 [M+ H]⁺ |
| II-37 | OCH₃ | N | CH | CH | CH | CH₂OCH(CH₃)₂ | CH₃ | H | cis | 3.156/426.1 [M + H]⁺ |
| II-38 | OCH₂CH₃ | N | CH | CH | CH | CH₂OCH(CH₃)₂ | CH₃ | H | cls | 3.188/440.2 [M + H]⁺ |
| II-39 | OCH₃ | N | CH | CH | CH | CH₂OCH(CH₃)₂ | CH₃ | H | trans | 3.156/426.1 [M + H]⁺ |
| II-40 | OCH₂CH₃ | N | CH | CH | CH | CH₂CH(OH)CH₃ | CH₃ | H | cis | 2.780/426.1 [M + H]⁺ |
| II-41 | OCH₂CH₃ | N | CH | CH | CH | CH₂CH(OH)CH₂OH | CH₃ | H | cis | 2.423/442.1 [M + H]⁺ |
| II-42 | OCH₂CH₃ | N | CH | CH | CH | CH₂CH(OH)CH₃ | CH₃ | H | trans | 2.680/426.1 [M + H]⁺ |
| II-43 | OCH₂CH₃ | N | CH | CH | CH | CH₂CH₂OH | CH₃ | H | cis | 2.537/412.1 [M + H]⁺ |
| II-44 | OCH₂CH₃ | N | CH | CH | CH | CH₂CH(OCH₃)₂ | CH₃ | H | cis | 3.000/424.1 [M − OCH₃]⁺ |
| II-45 | OCH₂CH₃ | N | CH | CH | CH | CH₂CH(OCH₃)₂ | CH₃ | H | trans | 2.994/424.1 [M − OCH₃]⁺ |
| II-46 | NO₂ | CH | CH | CH | CH | CH₂CO₂H | CH₃ | H | cis | 2.794/426.1 [M + H]⁺ |
| II-47 | OCH₂CH₃ | N | CH | CH | CH | CH₂CH₂OC₂H₅ | CH₃ | H | cis | 3.064/440.3 [M + H]⁺ |

^{*)}cis/trans refers to the position of the (hetero)arylalkyl group and the benzyl group of the diketopiperazine skeleton

### USE EXAMPLES

The herbicidal activity of the compounds of the formula I was demonstrated by the following greenhouse experiments:

The culture containers used were plastic flowerpots containing loamy sand with approximately 5.8% of humus as the substrate. The seeds of the test plants were sown separately for each species.

For the pre-emergence treatment, the active compounds, which had been suspended or emulsified in water, were applied directly after sowing by means of finely distributing nozzles. The containers were irrigated gently to promote germination and growth and subsequently covered with transparent plastic hoods until the plants had rooted. This cover caused uniform germination of the test plants, unless this has been impaired by the active compounds.

For the post-emergence treatment, the test plants were first grown to a height of 3 to 15 cm, depending on the plant habit, and then treated with the active compounds which had been suspended or emulsified in water. For this purpose, the test plants were either sown directly and grown in the same containers, or they were first grown separately as seedlings and transplanted into the test containers a few days prior to treatment.

Depending on the species, the plants were kept at 10-25° C. or 20-35° C. The test period extended over 2 to 4 weeks. During this time, the plants were tended, and their response to the individual treatments was evaluated.

Evaluation was carried out using a scale from 0 to 100. 100 means no emergence of the plants, or complete destruction of at least the aerial moieties, and 0 means no damage, or normal course of growth. A good herbicidal activity is given at values of at least 70 and a very good herbicidal activity is given at values of at least 85.

The plants used in the greenhouse experiments belonged to the following species:

| Bayer code | Scientific name | Common name |
|---|---|---|
| ABUTH | Abutilon theophrasti | China jute |
| ALOMY | Alopecurus myosuroides | blackgrass |
| AMARE | Amaranthus retoflexus | redroot pigweed |
| APESV | Apera spica-venti | windgrass |
| CHEAL | Chenopodium album | white goosefoot |
| ECHCG | Echinochloa crus-galli | barnyardgrass |
| LOLMU | Lolium multiflorum | Italian ryegrass |
| SETIT | Setaria italica | Italian millet |
| SETVI | Setaria viridis | green foxtail |
| SETFA | Setaria faberi | giant foxtail |

### EXAMPLES 1-12

### Individual Active Compounds

1) At an application rate of 1.0 kg/ha, the active compounds I-7 and I-25, respectively applied by the post-emergence method, showed very good herbicidal activity against ABUTH.

2) At an application rate of 0.5 kg/ha, the active compounds I-10 and I-21, respectively, and, at an application rate of 1.0 kg/ha, the active compound I-2, applied by the post-emergence method, showed very good herbicidal activity against AMARE.

3) At an application rate of 0.5 kg/ha, the active compounds I-10, I-14, I-24, I-18, I-19, I-26, I-34, I-38, I-39, I-40, I-53, I-54, I-56, I-57, I-72, I-76, I-81, I-85, I-91, I-92, I-94, I-95, I-103, II-1, II-14, II-15, II-31, II-32, II-33 and II-34, respectively, at 0.3 kg/ha, the active compound II-26 and, at 0.25 kg/ha, the active compound I-83, applied by the pre-emergence method, showed very good and the active compounds I-48 and I-49, respectively, showed good herbicidal activity against APESV.

4) At an application rate of 1.0 kg/ha, the active compounds I-5 and I-26, respectively, applied by the pre-emergence method, showed good and the active compounds I-4, I-5, I-9, I-12, I-13, I-16 and I-42 very good herbicidal activity against APESV.

5) At an application rate of 0.5 kg/ha, the active compound I-21, applied by the post-emergence method, showed very good herbicidal activity against CHEAL.

6) At an application rate of 1.0 kg/ha, the active compounds I-4, I-5, I-9, I-12, I-13, I-14, I-16, I-26 and I-42, respectively and, at an application rate of 0.5 kg/ha, the active compounds I-14, I-18, I-19, I-24, I-26, I-34, I-38, I-39, I-40, I-49, I-53, I-54, I-56, I-63, I-72, I-79, I-81, I-84, I-85, I-86, I-91, I-92, I-93, I-94, I-95, I-103, II-1, II-14, II-15, II-31, II-32, II-33 and II-34, respectively, at 0.3 kg/ha, the active compound II-26 and, at 0.25 kg/ha, the active compounds I-83 and II-27, respectively, applied by the pre-emergence method, showed very good herbicidal activity against ECHCG.

7) At an application rate of 1.0 kg/ha, the active compound I-19, applied by the post-emergence method, showed very good herbicidal activity against ECHCG.

8) At an application rate of 1.0 kg/ha, the active compounds I-4, I-5, I-12, I-13, I-14, I-16 and I-42, respectively and, at an application rate of 0.5 kg/ha, the active compounds I-14, I-19, I-24, I-48, I-49, I-53, I-54, I-56, I-57, I-63, I-72, I-76, I-79, I-81, I-84, I-85, I-86, I-91, I-92, I-93, I-94, I-95, I-103, II-1, II-14, II-15, II-31, II-33 and II-34, respectively, at 0.3 kg/ha, the active compound II-26 and, at 0.25 kg/ha, the active compounds I-83 and II-27, respectively, applied by the pre-emergence method, showed very good herbicidal activity against SETFA.

9) At an application rate of 1.0 kg/ha, the active compound I-25, applied by the post-emergence method, showed very good herbicidal activity against SETFA.

10) At an application rate of 1.0 kg/ha, the active compound I-19, applied by the post-emergence method, showed very good herbicidal activity against SETIT.

11) At an application rate of 1.0 kg/ha, the active compound I-5 and, at an application rate of 0.5 kg/ha, the active compound I-24, applied by the pre-emergence method, showed very good herbicidal activity against SETVI.

12) At an application rate of 0.5 kg/ha, the active compounds I-3 and I-21 and, at an application rate of 1.0 kg/ha, the active compound I-2, applied by the post-emergence method, showed very good herbicidal activity against SETVI.

### EXAMPLES 13-14

### Active Compound Combinations

The respective stated components A and B were formulated as a 5% by weight strength or 10% by weight strength emulsion concentrate, or a commercial formulation of component B was used, and, with addition of the amount of solvent system, introduced into the spray liquor used for applying the active compound. In the examples, the solvent used was water.

The test period extended over 20 or 21 days. During this time, the plants were tended, and their reactions to the treatments with the active compounds were monitored.

In the examples below, using the method of S. R. Colby (1967) “Calculating synergistic and antagonistic responses of herbicide combinations”, Weeds 15, p. 22ff., the value E, which is expected when the activity of the individual active compounds is only additive, was calculated.

E=X+Y−(X·Y/100)

where

X=percent activity using active compound A at an application rate a;

Y=percent activity using active compound B at an application rate b;

E=expected activity (in %) by A+B at application rates a+b.

If the value determined experimentally is higher than the value E calculated according to Colby, a synergistic effect is present.

The results of these tests are stated in the tables of use examples 13 and 14 below and demonstrate the synergistic effect of mixtures comprising at least one piperazine-dione compound of the formula I and at least one further herbicide.

Here, a.s.=active substance, based on 100% active compound. In the use examples 15 and 16, the values E calculated according to Colby are stated in brackets) ( ).

What was evaluated was the damage by the chemical compositions using a scale of from 0 to 100%, in comparison to the untreated control plants. 0 means no damage, and 100 means complete destruction of the plants.

13) Synergistic herbicidal activity of the active compound I-43 (5% EC) with atrazine (500 g/l ISC) by the pre-emergence method:

| | |
|---|---|
| | Herbicidal activity |
| | in % after |
| Application | 20 days against |

| Active compound | rate a.s. in g/ha | ALOMY | LOLMU |
|---|---|---|---|
| I-43 | 62.5 | 60 | 45 |
| I-43 | 125 | 85 | 90 |
| I-43 | 250 | 95 | 95 |
| atrazine | 125 | 35 | 0 |
| atrazine | 250 | 65 | 20 |
| I-43 + atrazine | 125 + 125 | 98 (90) | — |
| I-43 + atrazine | 250 + 125 | 98 (97) | — |
| I-43 + atrazine | 62.5 + 250 | 98 (86) | — |
| I-43 + atrazine | 125 + 250 | 100 (95) | — |
| I-43 + atrazine | 250 + 250 | 100 (98) | 98 (96) |

14) Synergistic herbicidal activity of the active compound II-26 (5% EC) with pyroxasulfone (100 g/l ISC) by the pre-emergence method:

| | | Herbicidal activity | | |
|---|---|---|---|---|
| | Application | in % after 20 days against | | |
| Active compound | rate a.s. in g/ha | ALOMY | LOLMU | SETFA |
| II-26 | 31.25 | 30 | 0 | 10 |
| II-26 | 62.5 | 60 | 15 | — |
| II-26 | 125 | 90 | 98 | 75 |
| II-26 | 250 | 95 | | 100 |
| pyroxasulfone | 3.12 | 25 | 15 | 0 |
| pyroxasulfone | 6.25 | — | — | 25 |
| pyroxasulfone | 12.5 | 95 | 75 | — |
| pyroxasulfone | 25 | 95 | 80 | 95 |
| II-26 + | 31.25 + 3.12 | 65 (48) | — | — |
| pyroxasulfone | | | | |
| II-26 + | 62.5 + 3.12 | 85 (70) | — | — |
| pyroxasulfone | | | | |
| II-26 + | 125 + 3.12 | 98 (93) | — | — |
| pyroxasulfone | | | | |
| II-26 + | 125 + 6.25 | — | — | 90 (81) |
| pyroxasulfone | | | | |
| II-26 + | 62.5 + 12.5 | — | 90 (79) | — |
| pyroxasulfone | | | | |

1. A compound of formula I 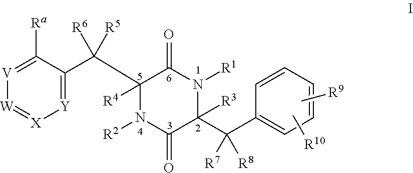
in which
R^{a} is halogen, CN, NO₂, C₁-C₄-alkyl, C₃-C₆-cycloalkyl, C₁-C₄-haloalkyl, C₁-C₄-alkoxy, C₁-C₄-thioalkyl, C₁-C₄-haloalkoxy, C₁-C₄-halothioalkyl, O—Z—C₃-C₆-cycloalkyl, S(O)ₙR^{y}, C₂-C₆-alkenyl, C₃-C₆-cycloalkenyl, C₃-C₆-alkenyloxy, C₃-C₆-thioalkenyl, C₂-C₆-alkynyl, C₃-C₆-alkynyloxy, C₃-C₆-thioalkynyl, NR^{A}R^{B}, tri-C₁-C₄-alkylsilyl, Z—C(═O)—R^{a1}, Z—C(═S)—R^{a1}, Z—C(═N—OR^{A})—R^{a1}, Z—C[═N(O)—R^{A}]—R^{a1}, Z—P(═O)(R^{a1})₂, or a 3- to 7-membered monocyclic or 9- or 10-membered bicyclic saturated, unsaturated or aromatic heterocycle which is attached via carbon or nitrogen, which contains 1, 2, 3 or 4 heteroatoms selected from the group consisting of O, N and S and which may be partially or fully substituted by groups R^{aa} and/or R^{a1} and/or may be fused to a further saturated, unsaturated or aromatic carbocyclic or heterocyclic ring,
R^{y} is C₁-C₆-alkyl, C₃-C₄-alkenyl, C₃-C₄-alkynyl, NR^{A}R^{B} or C₁-C₄-haloalkyl and n is 0, 1 or 2;
R^{A}, R^{B} independently of one another are hydrogen, C₁-C₆-alkyl, C₃-C₆-alkeny, C₃-C₆-alkynyl, C₃-C₆-cycloalkyl, C₁-C₆-alkylcarbonyl, C₃-C₆-cycloalkylcarbonyl, C₃-C₆-alkenylcarbonyl, C₃-C₆-cycloalkenylcarbonyl or C₃-C₆-alkynylcarbonyl; or
R^{A}, R^{B} together with the nitrogen atom to which they are attached may form a five- or six-membered saturated or partially or fully unsaturated ring which, in addition to carbon atoms, may contain 1, 2 or 3 heteroatoms selected from the group consisting of O, N and S, which ring may be substituted by 1 to 3 groups R^{aa};
Z is a covalent bond, C₁-C₄-alkylene, C₂-C₆-alkenyl or C₂-C₆-alkynyl;
R^{a1} is hydrogen, OH, C₁-C₈-alkyl, C₁-C₄-haloalkyl, C₃-C₆-cycloalkyl, C₂-C₈-alkenyl, C₅-C₆-cycloalkenyl, C₂-C₈-alkynyl, C₁-C₆-alkoxy, C₁-C₄-haloalkoxy, C₃-C₈-alkenyloxy, C₃-C₈-alkynyloxy, NH₂, C₁-C₆-alkylamino, [di-(C₁-C₆)-alkyl]amino, C₁-C₆-alkoxyamino, C₁-C₆-alkylsulfonylamino, C₁-C₆-alkylaminosulfonylamino, [di-(C₁-C₆)-alkylamino]sulfonylamino, C₃-C₆-alkenylamino, C₃-C₆-alkynylamino, N—(C₂-C₆-alkenyl)-N—(C₁-C₆-alkyl)amino, N—(C₂-C₆-alkynyl)-N—(C₁-C₆-alkyl)amino, N—(C₁-C₆-alkoxy)-N—(C₁-C₆-alkyl)amino, N—(C₂-C₆-alkenyl)-N—(C₁-C₆-alkoxy)amino, N—(C₂-C₆-alkynyl)-N—(C₁-C₆-alkoxy)amino, C₁-C₆-alkylsulfonyl, tri-C₁-C₄-alkylsilyl, phenyl, phenoxy, phenylamino or a 5- or 6-membered monocyclic or 9- or 10-membered bicyclic heterocycle which contains 1, 2, 3 or 4 heteroatoms selected from the group consisting of O, N and S, where the cyclic groups are unsubstituted or substituted by 1, 2, 3 or 4 groups R^{aa};
R^{aa} is halogen, OH, CN, NO₂, C₁-C₄-alkyl, C₁-C₄-haloalkyl, C₁-C₄-alkoxy, C₁-C₄-haloalkoxy, S(O)ₙR^{y}, Z—C(═O)—R^{a1}, Z—C(═S)—R^{a1}, Z—C(═N—OR^{A})—R^{al}, Z—C(═N(O)—R^{A})—R^{a1}, oxo (═O) or tri-C₁-C₄-alkylsilyl;
R¹ is OH, CN, C₅-C₁₂-alkyl, C₅-C₁₂-alkenyl, C₅-C₁₂-alkynyl, C₁-C₄-alkoxy, C₃-C₆-cycloalkyl, C₅-C₆-cycloalkenyl, CH₂CH═CHCH₃, CH₂CH₂CH═CH₂, CH₂C(CH₃)═CH₂, CH₂CH═C(CH₃)₂, CH₂CECCH₃, CH₂CH₂CCH, NR^{A}R^{B}, S(O)ₙR^{y}, S(O)ₙNR^{A}R^{B}, CO—C₁-C₂-chloroalkyl, CONR^{A}R^{B}, phenyl or a 5- or 6-membered monocyclic or 9- or 10-membered bicyclic saturated, partially unsaturated or aromatic heterocycle, which contains 1, 2, 3 or 4 heteroatoms selected from the group consisting of O, N and S, where the cyclic groups are attached via Z¹ and are unsubstituted or substituted by 1, 2, 3 or 4 groups R^{aa}, and also the following partially or fully R^{aa}-substituted groups: C₁-C₄-alkyl, C₃-C₄-alkenyl, and C₃-C₄-alkynyl;
Z¹ is carbonyl of a group Z;
where in groups R¹ and R^{a} and their sub-substituents the carbon chains and/or the cyclic groups may carry 1, 2, 3 or 4 substituents R^{aa} and/or R^{a1};
R² is C₁-C₄-alkyl, C₃-C₄-alkenyl or C₃-C₄-alkynyl;
R³ is OH, NH₂, C₁-C₄-alkyl, C₃-C₆-cycloalkyl, C₃-C₆-alkenyl, C₃-C₆-alkynyl, C₁-C₄-hydroxyalkyl, C₁-C₄-cyanoalkyl, C₁-C₄-haloalkyl, C₁-C₄-alkoxy-C₁-C₄-alkyl or C(═O)R¹¹;
R¹¹ is hydrogen, C₁-C₄-alkyl, C₁-C₄-haloalkyl, C₁-C₄-alkoxy or C₁-C₄-haloalkoxy;
R⁴ is hydrogen, halogen, C₁-C₄-alkyl or C₁-C₄-haloalkyl, or R⁴ and R⁵ together are a covalent bond;
R⁵, R⁶, R⁷, R⁸ independently of one another are hydrogen, halogen, CN, NO₂, C₁-C₄-alkyl, C₁-C₄-haloalkyl, C₂-C₆-alkenyl, C₂-C₆-alkynyl, C₁-C₄-alkoxy, C₁-C₄-haloalkoxy, C₃-C₆-cycloalkyl or C₃-C₆-cycloalkenyl;
R⁹, R¹⁰ independently of one another are hydrogen, halogen, OH, haloalkyl, NR^{A}R^{B}, NR^{A}C(O)R⁹¹, CN, NO₂, C₁-C₄-alkyl, C₁-C₄-haloalkyl, C₂-C₄-alkenyl, C₃-C₆-alkynyl, C₁-C₄-alkoxy, C₁-C₄-haloalkoxy, O—C(O)R⁹¹, phenoxy or benzyloxy, where in groups R⁹ and R¹⁰ the carbon chains and/or the cyclic groups may carry 1, 2, 3 or 4 substituents R^{aa};
R⁹¹ is C₁-C₄-alkyl or NR^{A}R^{B};
V, W, X, Y are independently of one another N or C—R^{b}, R¹) independently of one another are hydrogen, CN, NO₂, halogen, C₁-C₄-alkyl, C₁-C₄-haloalkyl, C₂-C₄-alkenyl, C₃-C₆-alkynyl, C₁-C₄-alkoxy, C₁-C₄-haloalkoxy, benzyl or S(O)ₙR^{y}; or
R^{a} and/or R^{b} together with the group R^{a} or R^{b} attached to the adjacent ring carbon atom or with the adjacent ring nitrogen atom itself may also form a five- or six-membered saturated or partially or fully unsaturated ring which, in addition to carbon atoms, may contain 1, 2 or 3 heteroatoms selected from the group consisting of O, N and S, which ring may be substituted by 1 to 3 groups R^{aa} and/or may be fused to a further saturated, unsaturated or aromatic carbocyclic or heterocyclic ring;
is a double bond, or, if R^{a} forms a cycle with a nitrogen atom in position V, is a single bond;
with the proviso that, if from one to three groups of V, W, X, Y are N, R¹ is not C₅-C₆-alkyl, C₁-C₄-haloalkyl, CH₂CH═CHCH₃, CH₂CH₂CH═CH₂, CH₂C(CH₃)═CH₂, CH₂C≡CCH₃ or CH₂CH₂C≡CH;
or an agriculturally suitable salt thereof.

2. The compound of claim 1, in which
R^{a} is halogen, CN, NO₂, C₁-C₄-alkyl, C₃-C₆-cycloalkyl, C₁-C₄-haloalkyl, C₁-C₄-alkoxy, C₁-C₄-haloalkoxy, O—Z—C₃-C₆-cycloalkyl, S(O)ₙR^{y}, C₂-C₆-alkenyl, C₃-C₆-cycloalkenyl, C₃-C₆-alkenyloxy, C₂-C₆-alkynyl, C₃-C₆-alkynyloxy, C₃-C₆-thioalkynyl, NR^{A}R^{B}, tri-C₁-C₄-alkylsilyl, Z—P(═O)(R^{a1})₂, or a 3- to 7-membered monocyclic or 9- or 10-membered bicyclic saturated, unsaturated or aromatic heterocycle which is attached via carbon or nitrogen, which contains 1, 2, 3 or 4 heteroatoms selected from the group consisting of O, N and S and which may be partially or fully substituted by groups R^{aa} and/or R^{a1},
R^{A}, R^{B} independently of one another are hydrogen, C₁-C₆-alkyl, C₃-C₆-alkenyl or C₃-C₆-alkynyl; or
R^{A}, R^{B} together with the nitrogen atom to which they are attached may also form a five- or six-membered saturated or partially or fully unsaturated ring which, in addition to carbon atoms, may contain 1, 2 or 3 heteroatoms selected from the group consisting of O, N and S, which ring may be substituted by 1 to 3 groups R^{aa};
R^{aa} is halogen, OH, CN, NO₂, C₁-C₄-haloalkyl, C₁-C₄-alkoxy, C₁-C₄-haloalkoxy, S(O)ₙR^{y}, Z—C(═O)—R^{a1} or tri-C₁-C₄-alkylsilyl;
R^{b} independently of one another are hydrogen, CN, NO₂, halogen, C₁-C₄-alkyl, C₁-C₄-haloalkyl, C₂-C₄-alkenyl, C₃-C₆-alkynyl, C₁-C₄-alkoxy, C₁-C₄-haloalkoxy, benzyl or S(O)ₙR^{y}; or
R^{b} together with the group R^{a} or R^{b} attached to the adjacent carbon atom may also form a five- or six-membered saturated or partially or fully unsaturated ring which, in addition to carbon atoms, may contain 1, 2 or 3 heteroatoms selected from the group consisting of O, N and S, which ring may be substituted by 1 to 3 groups R^{aa};
is a double bond.

3. The compound of claim 1 in which R^{a} is halogen, cyano or nitro.

4. The compound of claim 1 in which R^{a} is C₁-C₄-alkyl, C₁-C₄-alkoxy, C₁-C₄-haloalkyl, C₁-C₄-haloalkoxy, S(O)ₙR^{y}, C₁-C₄-haloalkylthio or a 3- to 7-membered monocyclic or 9- or 10-membered bicyclic saturated, unsaturated or aromatic heterocycle which is attached via nitrogen, which contains 1, 2, 3 or 4 heteroatoms selected from the group consisting of O, N and S and which may be partially or fully substituted by groups R^{aa} and/or R^{a1}.

5. The compound of claim 1 in which R^{a} is chlorine, cyano, nitro, C₁-C₄-alkoxy, C₁-C₄-thioalkyl, trifluoromethyl or NR^{A}R^{B}, where R^{A} and R^{B} together with the nitrogen atom to which they are attached form an optionally substituted 6-membered saturated heterocycle which contains 1 or 2 heteroatoms selected from the group consisting of O and N.

6. The compound of claim 1 in which R⁴ and R⁵ together are a covalent bond.

7. The compound of claim 6 where the exo double bond at the piperazine ring has the (Z) configuration.

8. The compound of claim 1 which corresponds to the formula I.1 or I.2 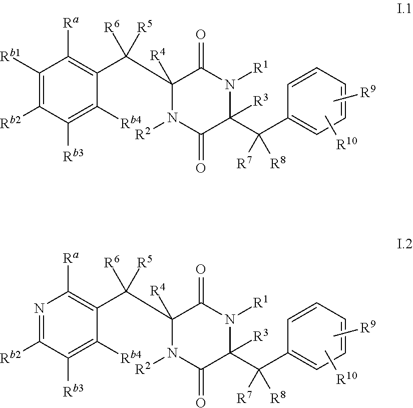
in which R^{b1}, R^{b2}, R^{b3}, and R^{b4} are a group R^{b}.

9. The compound of claim 1 in which R¹ is OH, CN, NH₂, OCH₃, CH₂CN, CH₂OCH₃, SO₂CH₃, CH₂-c-C₃H₅ or CH₂CH═CHCl.

10. The compound of claim 1 in which R² is methyl or ethyl.

11. The compound of claim 1 in which R³ is methyl or ethyl.

12. The compound of claim 1 in which R⁴ and R⁵ together are a covalent bond or are hydrogen and R⁶, R⁷ and R⁸ are hydrogen.

13. A composition comprising a herbicidally effective amount of at least one piperazine compound of the formula I or an agriculturally suitable salt thereof according to claim 1 and auxiliary compounds customary for formulating crop protection agents.

14. The composition according to claim 13 which comprises at least one further active compound.

15. A method for controlling unwanted vegetation which comprises applying a herbicidally effective amount of at least a compound of claim 1 to plants, their seed and/or their habitat and allowing said compound to act on said plants, seed, and/or habitat.

16. The method of claim 15, in which
R^{a} is halogen, CN, NO₂, C₁-C₄-alkyl, C₃-C₆-cycloalkyl, C₁-C₄-haloalkyl, C₁-C₄-alkoxy, C₁-C₄-haloalkoxy, O—Z—C₃-C₆-cycloalkyl, S(O)ₙR^{y}, C₂-C₆-alkenyl, C₃-C₆-cycloalkenyl, C₃-C₆-alkenyloxy, C₂-C₆-alkynyl, C₃-C₆-alkynyloxy, C₃-C₆-thioalkynyl, NR^{A}R^{B}, tri-C₁-C₄-alkylsilyl, Z—P(═O)(R^{a1})₂, or a 3- to 7-membered monocyclic or 9- or 10-membered bicyclic saturated, unsaturated or aromatic heterocycle which is attached via carbon or nitrogen, which contains 1, 2, 3 or 4 heteroatoms selected from the group consisting of O, N and S and which may be partially or fully substituted by groups R^{aa} and/or R^{a1},
R^{A}, R^{B} independently of one another are hydrogen, C₁-C₆-alkyl, C₃-C₆-alkenyl or C₃-C₆-alkynyl; or
R^{A}, R^{B} together with the nitrogen atom to which they are attached may also form a five- or six-membered saturated or partially or fully unsaturated ring which, in addition to carbon atoms, may contain 1, 2 or 3 heteroatoms selected from the group consisting of O, N and S, which ring may be substituted by 1 to 3 groups R^{aa};
R^{aa} is halogen, OH, CN, NO₂, C₁-C₄-alkyl, C₁-C₄-haloalkyl, C₁-C₄-alkoxy, C₁-C₄-haloalkoxy, S(O)ₙR^{y}, Z—C(═O)—R^{a1} or tri-C₁-C₄-alkylsilyl;
R^{b} independently of one another are hydrogen, CN, NO₂, halogen, C₁-C₄-alkyl, C₁-C₄-haloalkyl, C₂-C₄-alkenyl, C₃-C₆-alkynyl, C₁-C₄-alkoxy, C₁-C₄-haloalkoxy, benzyl or S(O)ₙR^{y}; or
R^{b} together with the group R^{a} or R^{b} attached to the adjacent carbon atom may also form a five- or six-membered saturated or partially or fully unsaturated ring which, in addition to carbon atoms, may contain 1, 2 or 3 heteroatoms selected from the group consisting of O, N and S, which ring may be substituted by 1 to 3 groups R^{aa};
is a double bond.

17. The method of claim 15, in which R^{a} is halogen, cyano or nitro.

18. The method of claim 15, in which R^{a} is C₁-C₄-alkyl, C₁-C₄-alkoxy, haloalkyl, C₁-C₄-haloalkoxy, S(O)ₙR^{y}, C₁-C₄-haloalkylthio or a 3- to 7-membered monocyclic or 9- or 10-membered bicyclic saturated, unsaturated or aromatic heterocycle which is attached via nitrogen, which contains 1, 2, 3 or 4 heteroatoms selected from the group consisting of O, N and S and which may be partially or fully substituted by groups R^{aa} and/or R^{a1}.

19. The method of claim 15, in which R^{a} is chlorine, cyano, nitro, C₁-C₄-alkoxy, C₁-C₄-thioalkyl, trifluoromethyl or NR^{A}R^{B}, where R^{A} and R^{B} together with the nitrogen atom to which they are attached form an optionally substituted 6-membered saturated heterocycle which contains 1 or 2 heteroatoms selected from the group consisting of O and N.

20. The method of claim 15, in which R⁴ and R⁵ together are a covalent bond.
